(19) United States
(12) Patent Application Publication
(10) Pub. No.: US 2015/0376114 A1
(43) Pub. Date: Dec. 31, 2015

(54) MATERIAL FOR ORGANIC ELECTROLUMINESCENT DEVICE AND ORGANIC ELECTROLUMINESCENT DEVICE INCLUDING THE SAME

(71) Applicant: SAMSUNG DISPLAY CO., LTD., Yongin-City (KR)

(72) Inventor: Naoya SAKAMOTO, Yokohama (JP)

(21) Appl. No.: 14/716,657

(22) Filed: May 19, 2015

(30) Foreign Application Priority Data
Jun. 30, 2014 (JP) 2014-133853

(51) Int. Cl. C07C 211/54; H01L 51/00; C07D 333/76; C09K 11/06; C07C 211/58; C07D 307/91

(52) U.S. Cl. CPC C07C 211/54; C07C 211/58; C07D 307/91; C07D 333/76; C09K 11/06; H01L 51/0059; H01L 51/006; H01L 51/0061; C09K 2211/1014; C09K 2211/1088; C09K 2211/1092; H01L 51/0052; H01L 51/0073; H01L 51/0074; H01L 51/5056

(57) ABSTRACT

A compound for an organic electroluminescent device and an organic electroluminescent device, the compound being represented by the following Formula 1:

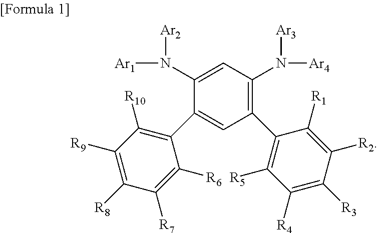

### CROSS-REFERENCE TO RELATED APPLICATION

Japanese Patent Application No. 2014-133853, filed on Jun. 30, 2014, in the Japanese Patent Office, and entitled: “Material For Organic Electroluminescent Device and Organic Electroluminescent Device Including The Same,” is incorporated by reference herein in its entirety.

### BACKGROUND

1. Field

Embodiments relate to a material for an organic electroluminescent device and an organic electroluminescent device including the same.

2. Description of the Related Art

Recently, the development of an organic electroluminescent display has been actively conducted. In addition, the development of an organic electroluminescent device, which is a self-emitting type device, has also been actively conducted.

As the organic electroluminescent device, a structure may include, e.g., an anode, a hole transport layer disposed on the anode, an emission layer disposed on the hole transport layer, an electron transport layer disposed on the emission layer and a cathode disposed on the electron transport layer.

In the organic electroluminescent device, holes and electrons injected from the anode and the cathode may recombine in the emission layer to produce excitons, and the excitons thus produced may transit to a ground state while emitting light.

### SUMMARY

Embodiments are directed to a material for an organic electroluminescent device and an organic electroluminescent device including the same.

The embodiments may be realized by providing a compound for an organic electroluminescent device, the compound being represented by the following Formula 1:

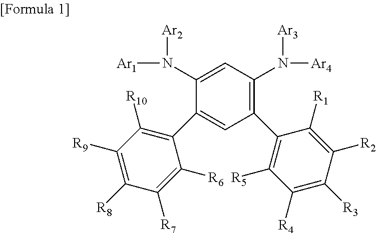

wherein, in Formula 1, Ar₁ to Ar₄ are each independently a substituted or unsubstituted aryl group or a substituted or unsubstituted heteroaryl group, at least one of Ar₁ to Ar₄ being a substituted or unsubstituted aryl group having at least 10 ring carbon atoms or a substituted or unsubstituted heteroaryl group having at least 10 ring carbon atoms, R₁ to R₁₀ are each independently a hydrogen atom, a substituted or unsubstituted aryl group, a substituted or unsubstituted heteroaryl group, a substituted or unsubstituted alkyl group, a substituted or unsubstituted alkoxy group, a halogen atom, an electron withdrawing group, or a deuterium atom, and R₁ to R₁₀ are separate or adjacent ones thereof are combined to each other to form a substituted or unsubstituted ring.

The aryl group having at least 10 ring carbon atoms may be a biphenyl group, a naphthyl group, a phenanthryl group, or a triphenylenyl group.

The aryl group having at least 10 ring carbon atoms may be a biphenyl group.

The heteroaryl group having at least 10 ring carbon atoms may be a dibenzoheterole group.

The dibenzoheterole group may be represented by the following Formula 2:

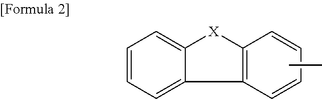

wherein, in Formula 2, X may be one of O, S, or Si(R₁₁)₂, in which each R₁₁ may be independently a hydrogen, a substituted or unsubstituted alkyl group, or a substituted or unsubstituted aryl group.

Ar₁ to Ar₄ may each independently be a substituted or unsubstituted aryl group having at least 10 ring carbon atoms or a substituted or unsubstituted heteroaryl group having at least 10 ring carbon atoms.

The compound may include one of the following Compounds 1-1 to 1-24:

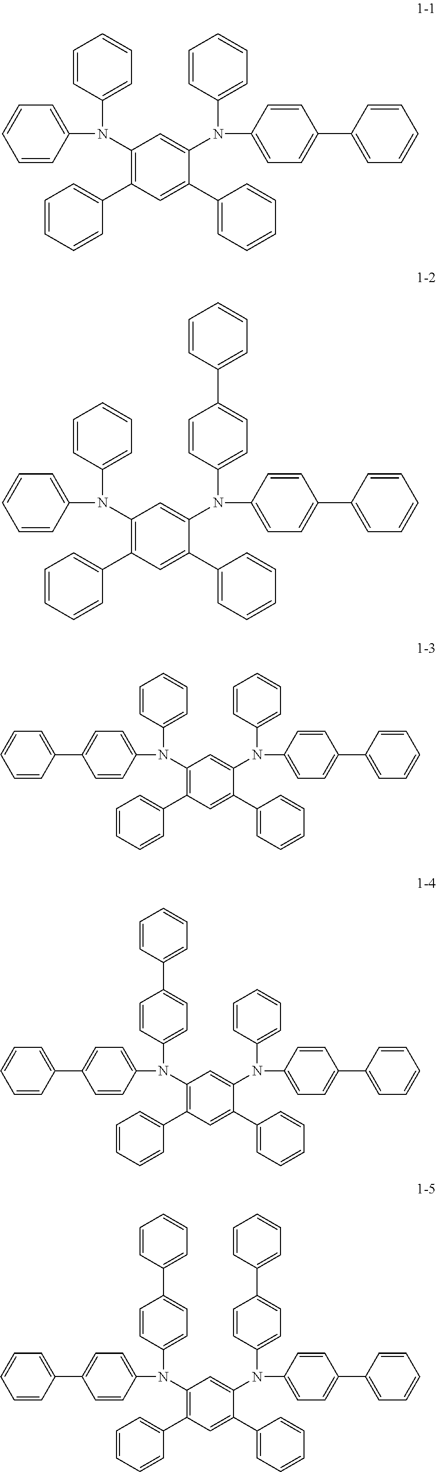 [SMILES: c1ccc(-c2ccc(N(c3ccccc3)c3cc(N(c4ccccc4)c4ccccc4)c(-c4ccccc4)cc3-c3ccccc3)cc2)cc1 ; c1ccc(-c2ccc(N(c3ccc(-c4ccccc4)cc3)c3cc(N(c4ccccc4)c4ccccc4)c(-c4ccccc4)cc3-c3ccccc3)cc2)cc1 ; c1ccc(-c2ccc(N(c3ccccc3)c3cc(N(c4ccccc4)c4ccc(-c5ccccc5)cc4)c(-c4ccccc4)cc3-c3ccccc3)cc2)cc1 ; c1ccc(-c2ccc(N(c3ccccc3)c3cc(N(c4ccc(-c5ccccc5)cc4)c4ccc(-c5ccccc5)cc4)c(-c4ccccc4)cc3-c3ccccc3)cc2)cc1 ; c1ccc(-c2ccc(N(c3ccc(-c4ccccc4)cc3)c3cc(N(c4ccc(-c5ccccc5)cc4)c4ccc(-c5ccccc5)cc4)c(-c4ccccc4)cc3-c3ccccc3)cc2)cc1]

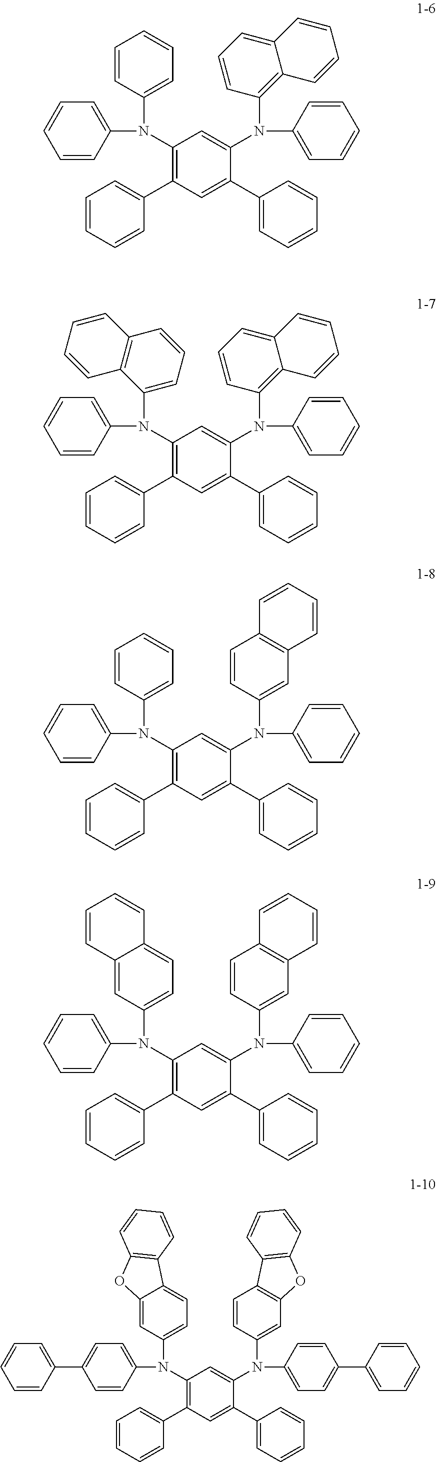

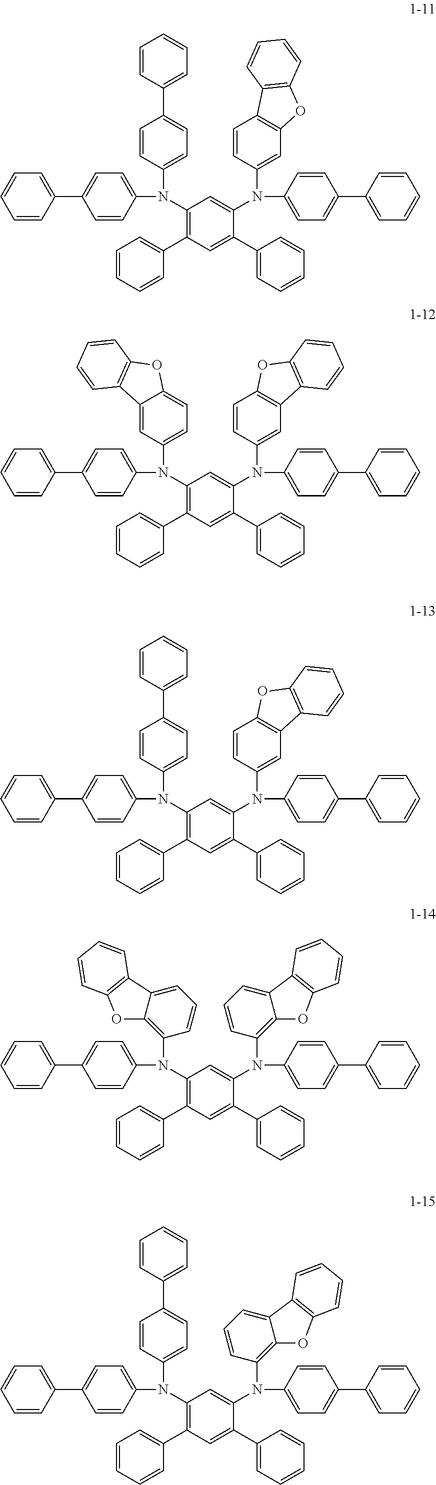

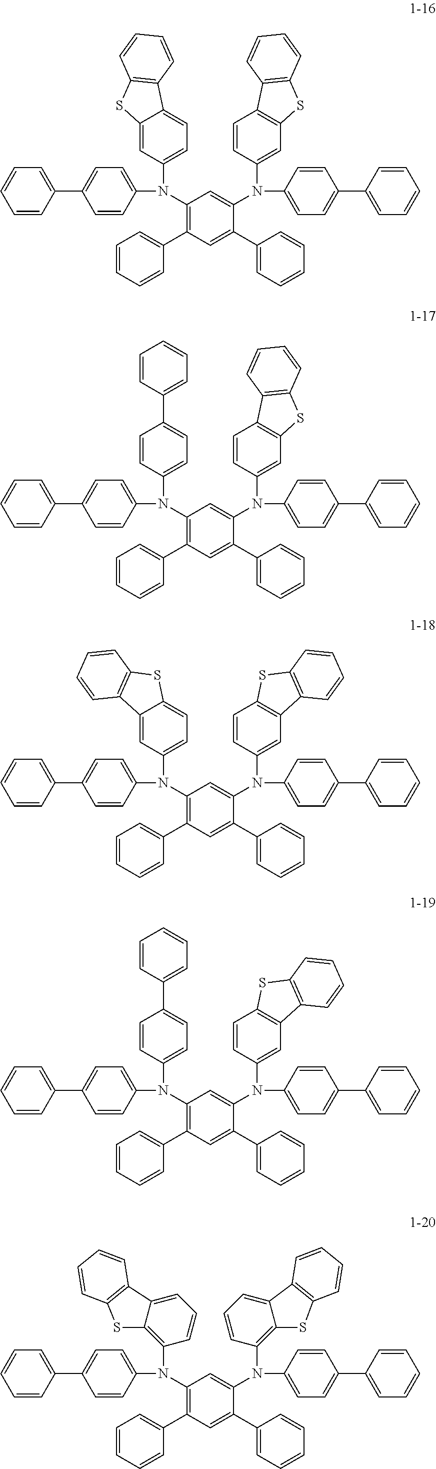

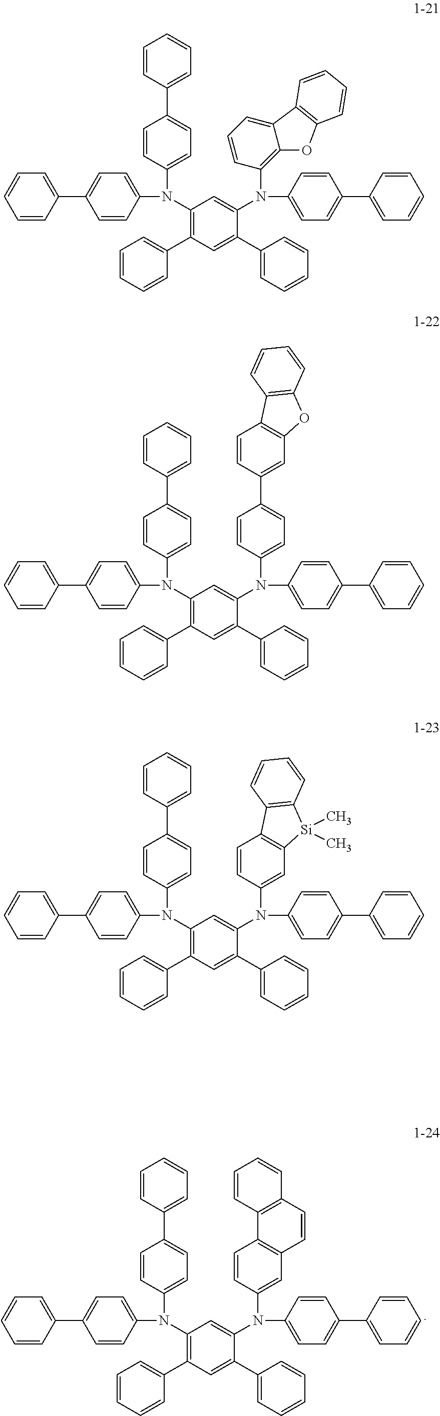 [SMILES: c1ccc(-c2ccc(N(c3ccc(-c4ccccc4)cc3)c3cc(N(c4ccc(-c5ccccc5)cc4)c4cccc5c4oc4ccccc45)c(-c4ccccc4)cc3-c3ccccc3)cc2)cc1 ; c1ccc(-c2ccc(N(c3ccc(-c4ccccc4)cc3)c3cc(N(c4ccc(-c5ccccc5)cc4)c4ccc(-c5ccc6c(c5)oc5ccccc56)cc4)c(-c4ccccc4)cc3-c3ccccc3)cc2)cc1 ; C[Si]1(C)c2ccccc2-c2ccc(N(c3ccc(-c4ccccc4)cc3)c3cc(N(c4ccc(-c5ccccc5)cc4)c4ccc(-c5ccccc5)cc4)c(-c4ccccc4)cc3-c3ccccc3)cc21 ; c1ccc(-c2ccc(N(c3ccc(-c4ccccc4)cc3)c3cc(N(c4ccc(-c5ccccc5)cc4)c4ccc5c(ccc6ccccc65)c4)c(-c4ccccc4)cc3-c3ccccc3)cc2)cc1]

The compound may include one of the following Compounds 1-25 to 1-46:

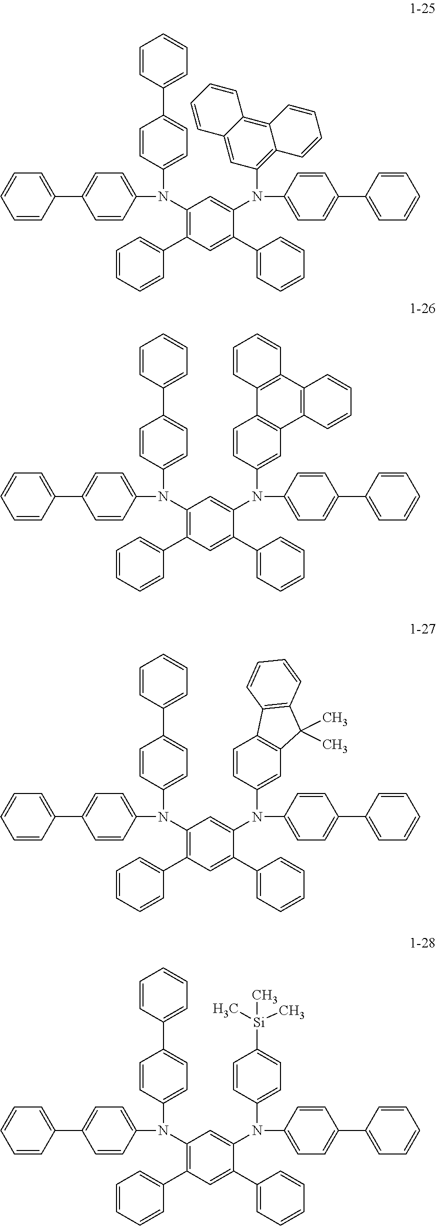 [SMILES: c1ccc(-c2ccc(N(c3ccc(-c4ccccc4)cc3)c3cc(N(c4ccc(-c5ccccc5)cc4)c4cc5ccccc5c5ccccc45)c(-c4ccccc4)cc3-c3ccccc3)cc2)cc1 ; c1ccc(-c2ccc(N(c3ccc(-c4ccccc4)cc3)c3cc(N(c4ccc(-c5ccccc5)cc4)c4ccc5c6ccccc6c6ccccc6c5c4)c(-c4ccccc4)cc3-c3ccccc3)cc2)cc1 ; CC1(C)c2ccccc2-c2ccc(N(c3ccc(-c4ccccc4)cc3)c3cc(N(c4ccc(-c5ccccc5)cc4)c4ccc(-c5ccccc5)cc4)c(-c4ccccc4)cc3-c3ccccc3)cc21 ; C[Si](C)(C)c1ccc(N(c2ccc(-c3ccccc3)cc2)c2cc(N(c3ccc(-c4ccccc4)cc3)c3ccc(-c4ccccc4)cc3)c(-c3ccccc3)cc2-c2ccccc2)cc1]

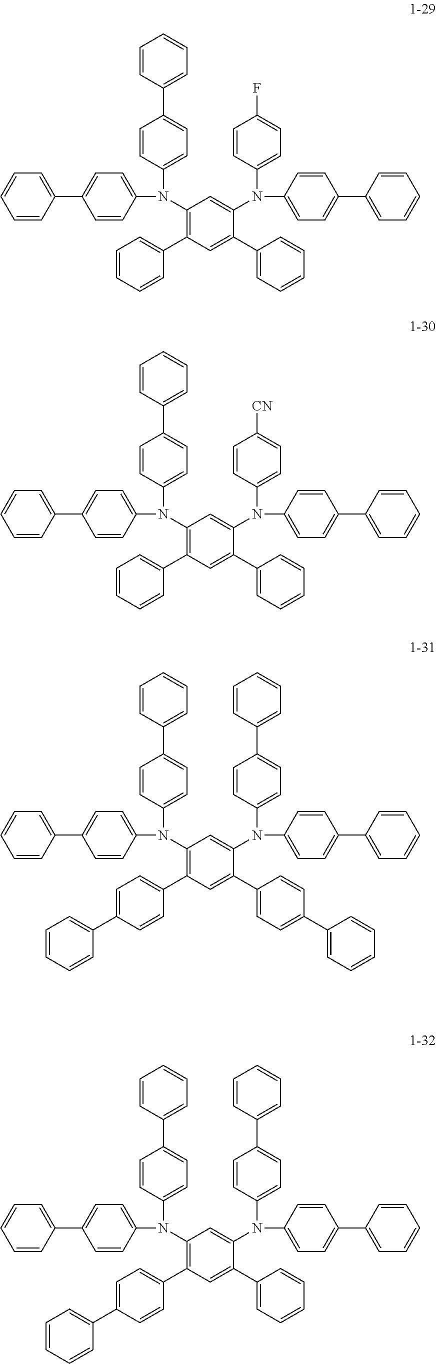 [SMILES: Fc1ccc(N(c2ccc(-c3ccccc3)cc2)c2cc(N(c3ccc(-c4ccccc4)cc3)c3ccc(-c4ccccc4)cc3)c(-c3ccccc3)cc2-c2ccccc2)cc1 ; N#Cc1ccc(N(c2ccc(-c3ccccc3)cc2)c2cc(N(c3ccc(-c4ccccc4)cc3)c3ccc(-c4ccccc4)cc3)c(-c3ccccc3)cc2-c2ccccc2)cc1 ; c1ccc(-c2ccc(-c3cc(-c4ccc(-c5ccccc5)cc4)c(N(c4ccc(-c5ccccc5)cc4)c4ccc(-c5ccccc5)cc4)cc3N(c3ccc(-c4ccccc4)cc3)c3ccc(-c4ccccc4)cc3)cc2)cc1 ; c1ccc(-c2ccc(-c3cc(-c4ccccc4)c(N(c4ccc(-c5ccccc5)cc4)c4ccc(-c5ccccc5)cc4)cc3N(c3ccc(-c4ccccc4)cc3)c3ccc(-c4ccccc4)cc3)cc2)cc1]

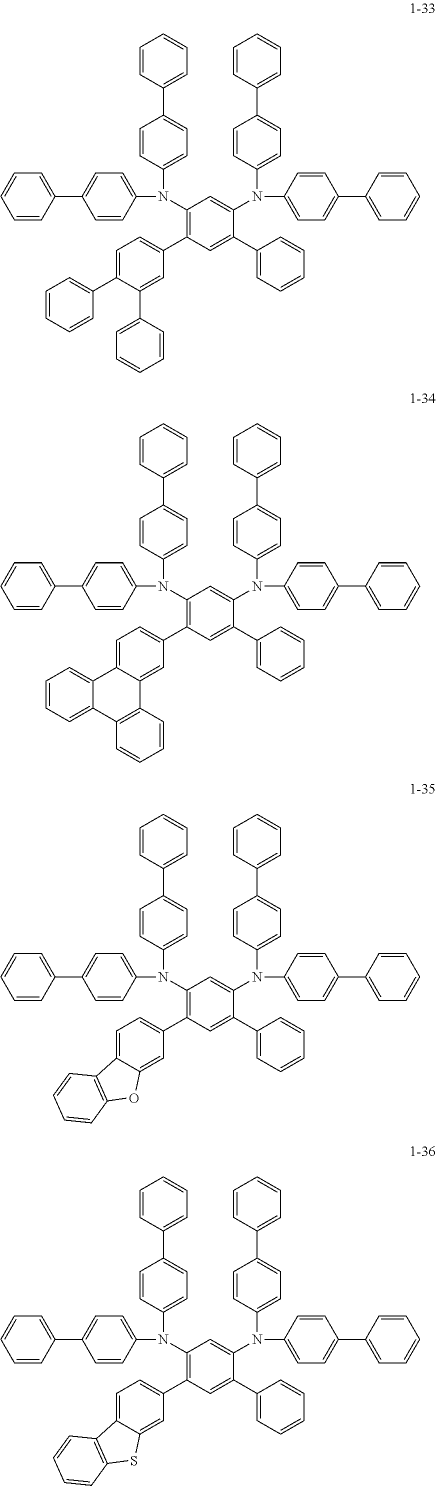

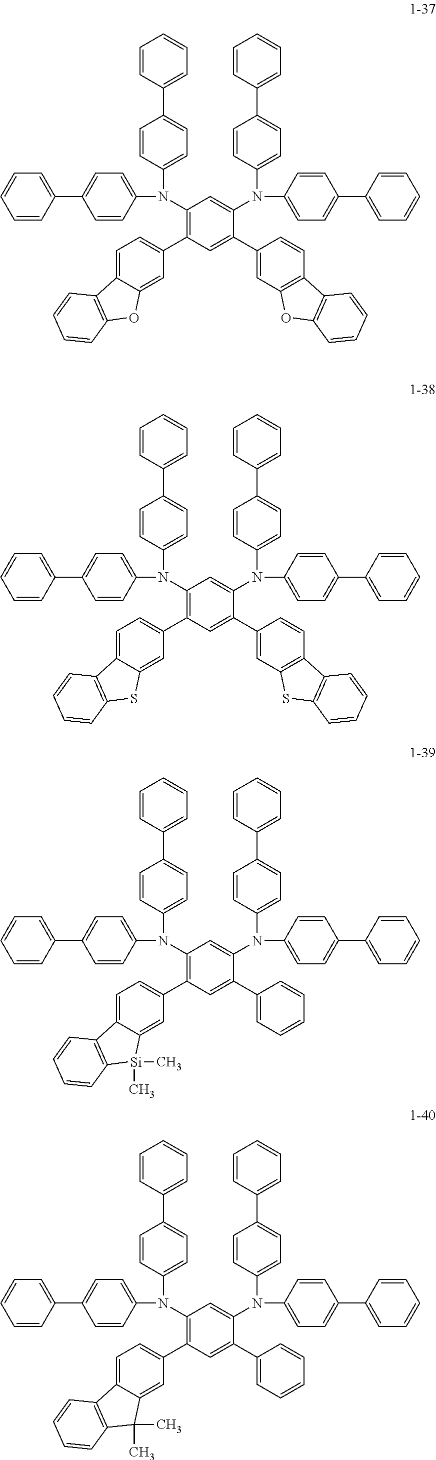

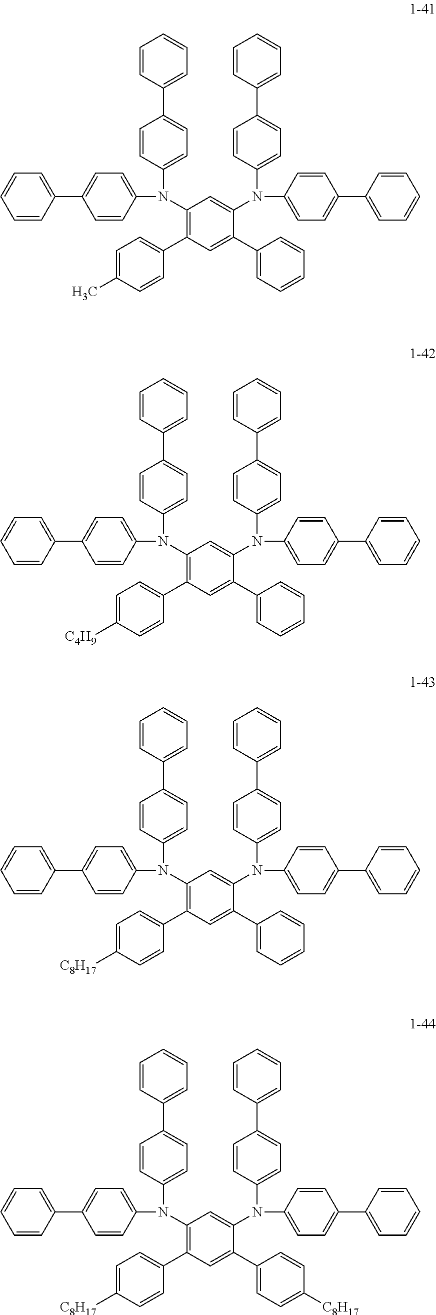

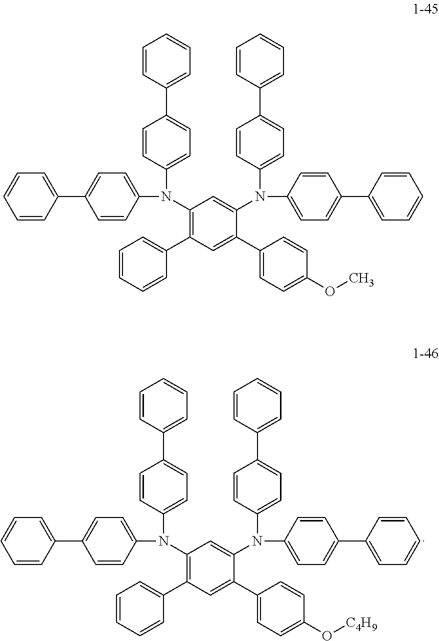

The compound may include one of the following Compounds 1-47 to 1-58:

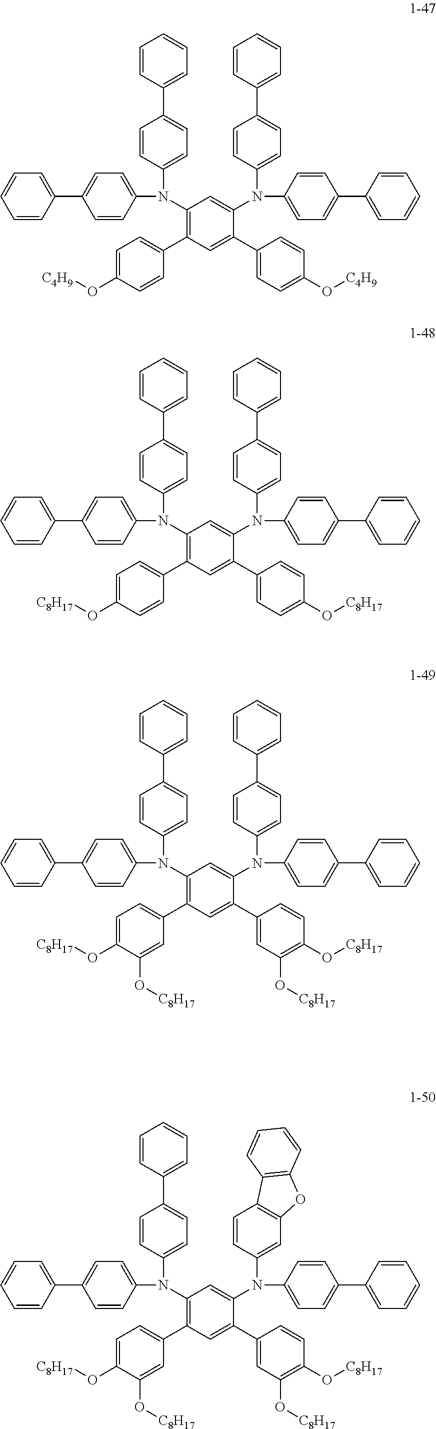

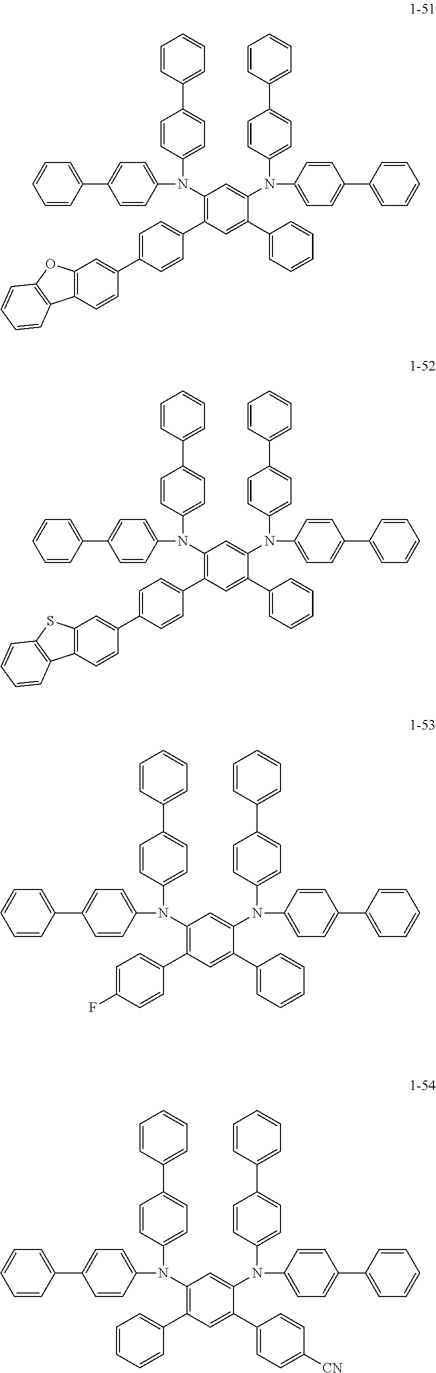

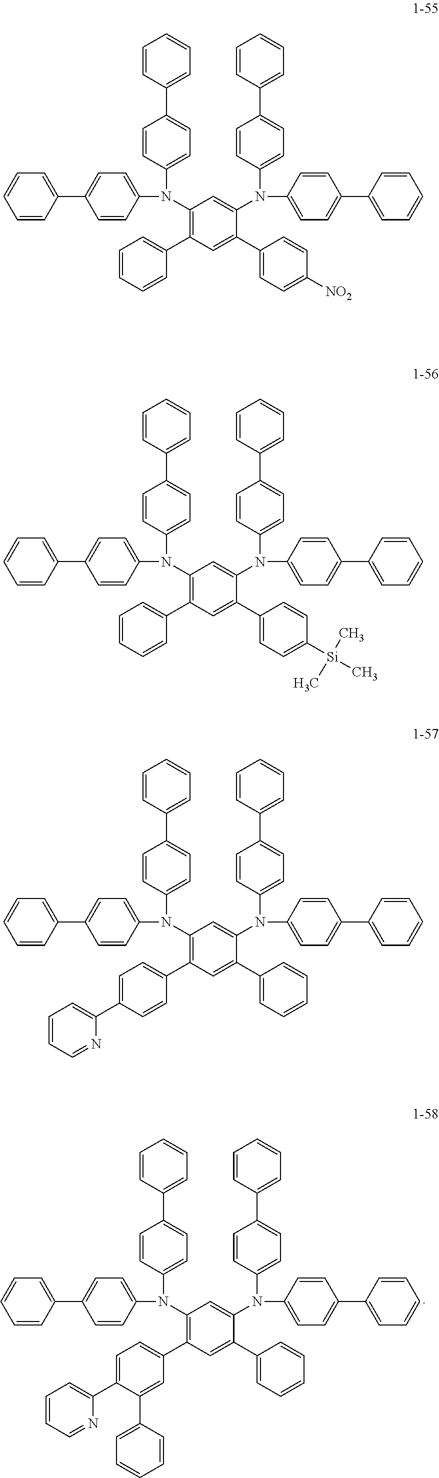

The embodiments may be realized by providing an organic electroluminescent device comprising a compound for an organic electroluminescent device, the compound being represented by the following Formula 1:

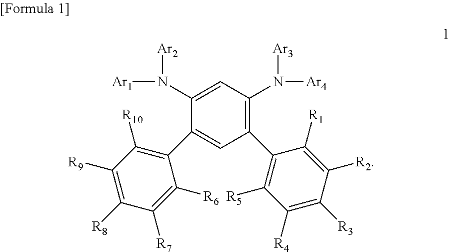

wherein, in Formula 1, Ar₁ to Ar₄ are each independently a substituted or unsubstituted aryl group or a substituted or unsubstituted heteroaryl group, at least one of Ar₁ to Ar₄ being a substituted or unsubstituted aryl group having at least 10 ring carbon atoms or a substituted or unsubstituted heteroaryl group having at least 10 ring carbon atoms, R₁ to R₁₀ are each independently a hydrogen atom, a substituted or unsubstituted aryl group, a substituted or unsubstituted heteroaryl group, a substituted or unsubstituted alkyl group, a substituted or unsubstituted alkoxy group, a halogen atom, an electron withdrawing group, or a deuterium atom, and R₁ to R₁₀ are separate or adjacent ones thereof are combined to each other to form a substituted or unsubstituted ring.

The aryl group having at least 10 ring carbon atoms may be a biphenyl group, a naphthyl group, a phenanthryl group, or a triphenylenyl group.

The aryl group having at least 10 ring carbon atoms may be a biphenyl group.

The heteroaryl group having at least 10 ring carbon atoms may be a dibenzoheterole group.

The dibenzoheterole group may be represented by the following Formula 2:

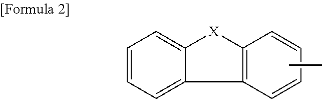

wherein, in Formula 2, X may be one of O, S, or Si(R₁₁)₂, in which each R₁₁ may be independently a hydrogen, a substituted or unsubstituted alkyl group, or a substituted or unsubstituted aryl group.

Ar₁ to Ar₄ may each independently be a substituted or unsubstituted aryl group having at least 10 ring carbon atoms or a substituted or unsubstituted heteroaryl group having at least 10 ring carbon atoms.

The compound may include one of the following Compounds 1-1 to 1-24:

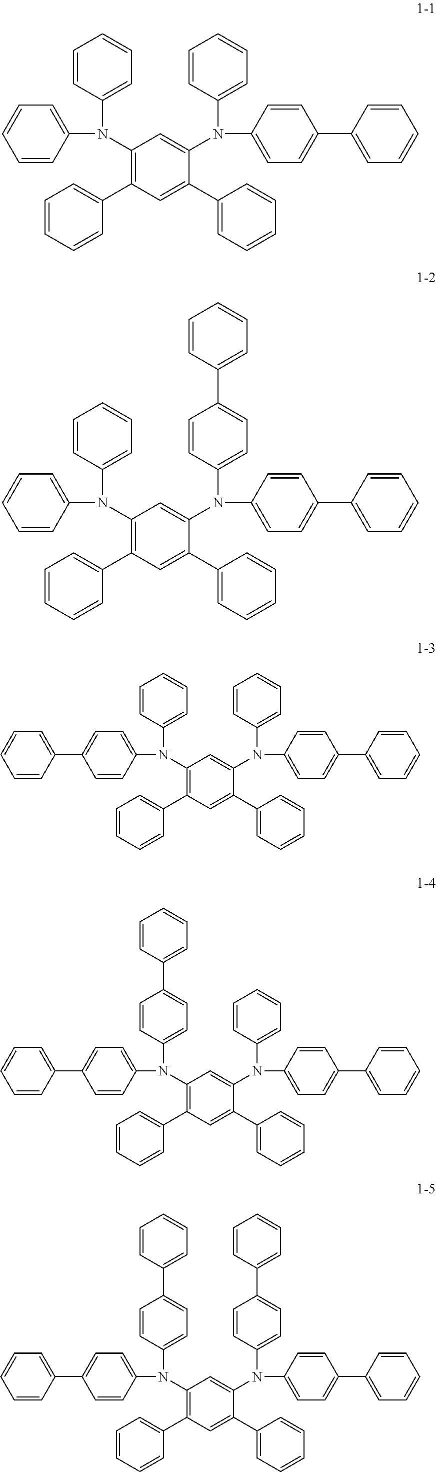 [SMILES: c1ccc(-c2ccc(N(c3ccccc3)c3cc(N(c4ccccc4)c4ccccc4)c(-c4ccccc4)cc3-c3ccccc3)cc2)cc1 ; c1ccc(-c2ccc(N(c3ccc(-c4ccccc4)cc3)c3cc(N(c4ccccc4)c4ccccc4)c(-c4ccccc4)cc3-c3ccccc3)cc2)cc1 ; c1ccc(-c2ccc(N(c3ccccc3)c3cc(N(c4ccccc4)c4ccc(-c5ccccc5)cc4)c(-c4ccccc4)cc3-c3ccccc3)cc2)cc1 ; c1ccc(-c2ccc(N(c3ccccc3)c3cc(N(c4ccc(-c5ccccc5)cc4)c4ccc(-c5ccccc5)cc4)c(-c4ccccc4)cc3-c3ccccc3)cc2)cc1 ; c1ccc(-c2ccc(N(c3ccc(-c4ccccc4)cc3)c3cc(N(c4ccc(-c5ccccc5)cc4)c4ccc(-c5ccccc5)cc4)c(-c4ccccc4)cc3-c3ccccc3)cc2)cc1]

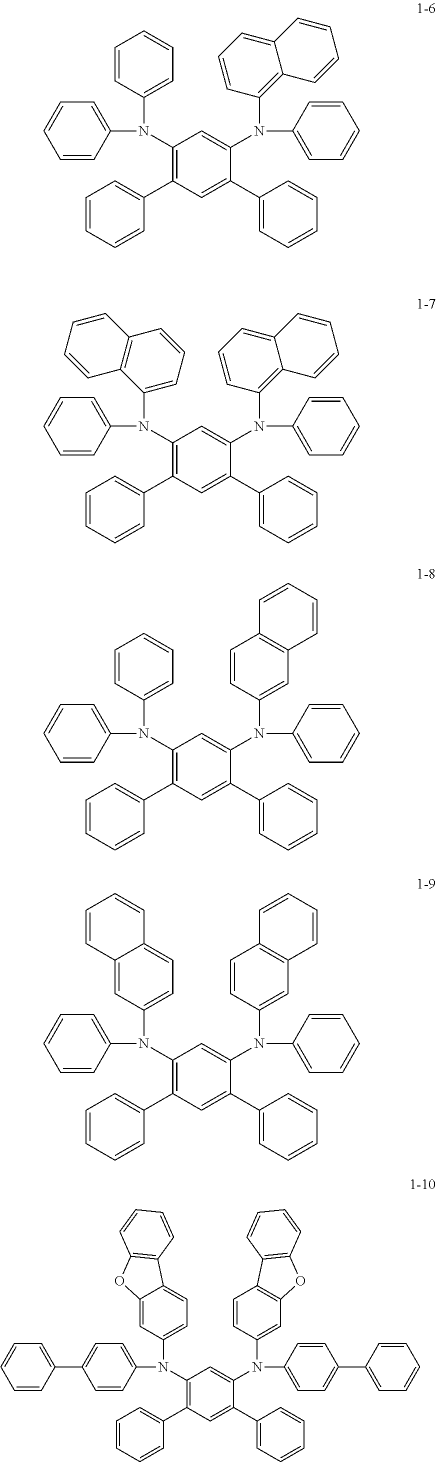

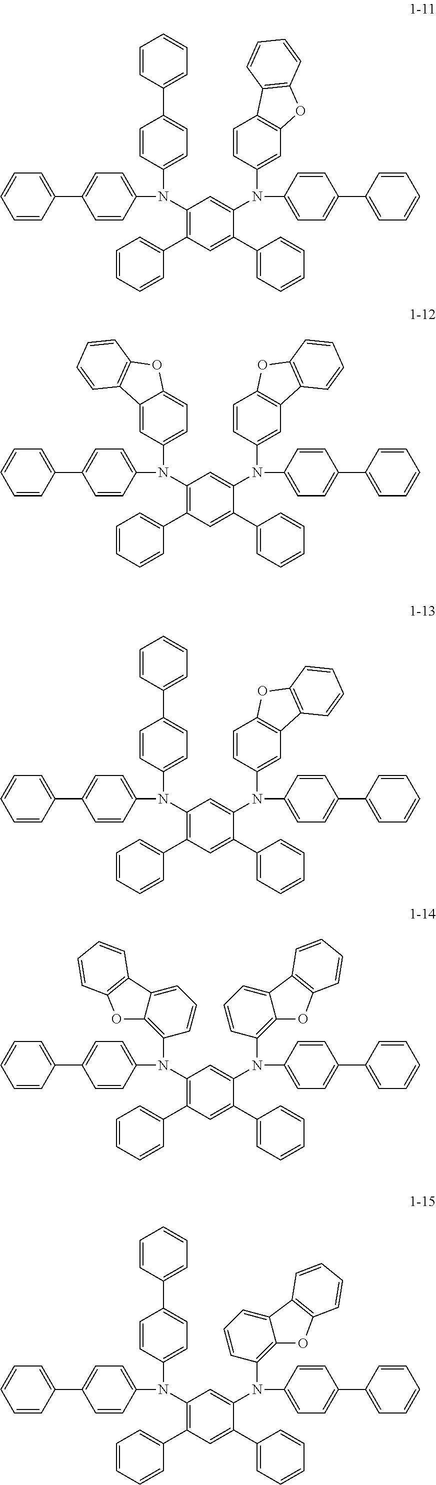

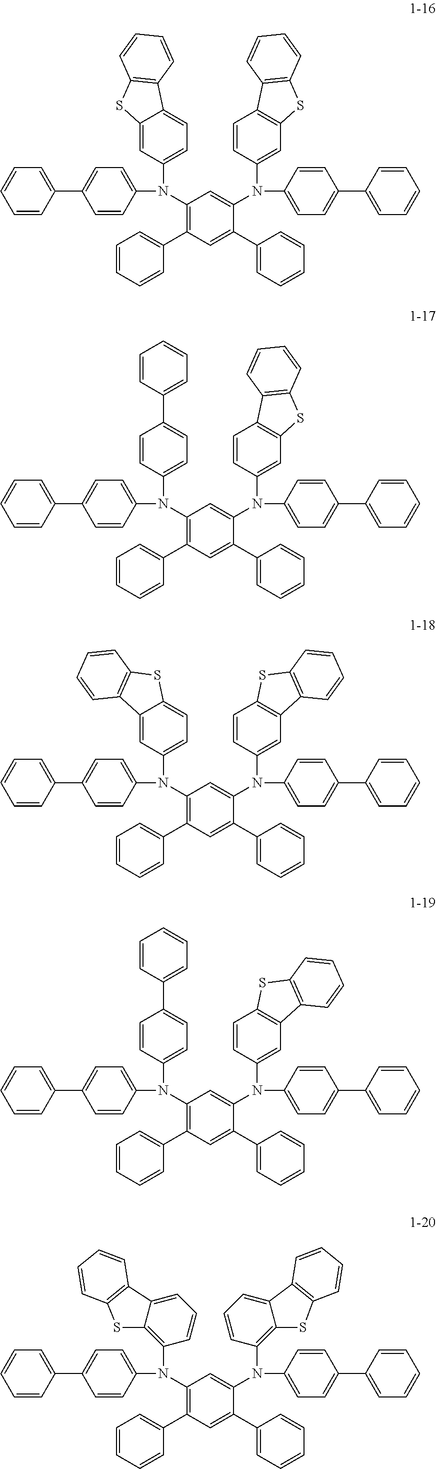

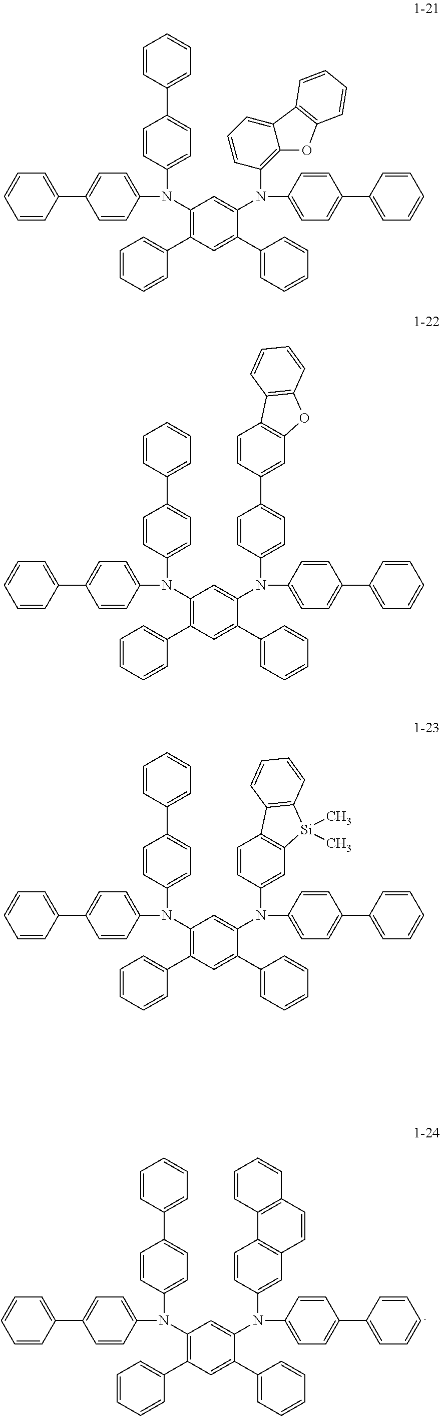 [SMILES: c1ccc(-c2ccc(N(c3ccc(-c4ccccc4)cc3)c3cc(N(c4ccc(-c5ccccc5)cc4)c4cccc5c4oc4ccccc45)c(-c4ccccc4)cc3-c3ccccc3)cc2)cc1 ; c1ccc(-c2ccc(N(c3ccc(-c4ccccc4)cc3)c3cc(N(c4ccc(-c5ccccc5)cc4)c4ccc(-c5ccc6c(c5)oc5ccccc56)cc4)c(-c4ccccc4)cc3-c3ccccc3)cc2)cc1 ; C[Si]1(C)c2ccccc2-c2ccc(N(c3ccc(-c4ccccc4)cc3)c3cc(N(c4ccc(-c5ccccc5)cc4)c4ccc(-c5ccccc5)cc4)c(-c4ccccc4)cc3-c3ccccc3)cc21 ; c1ccc(-c2ccc(N(c3ccc(-c4ccccc4)cc3)c3cc(N(c4ccc(-c5ccccc5)cc4)c4ccc5c(ccc6ccccc65)c4)c(-c4ccccc4)cc3-c3ccccc3)cc2)cc1]

The compound may include one of the following Compounds 1-25 to 1-46:

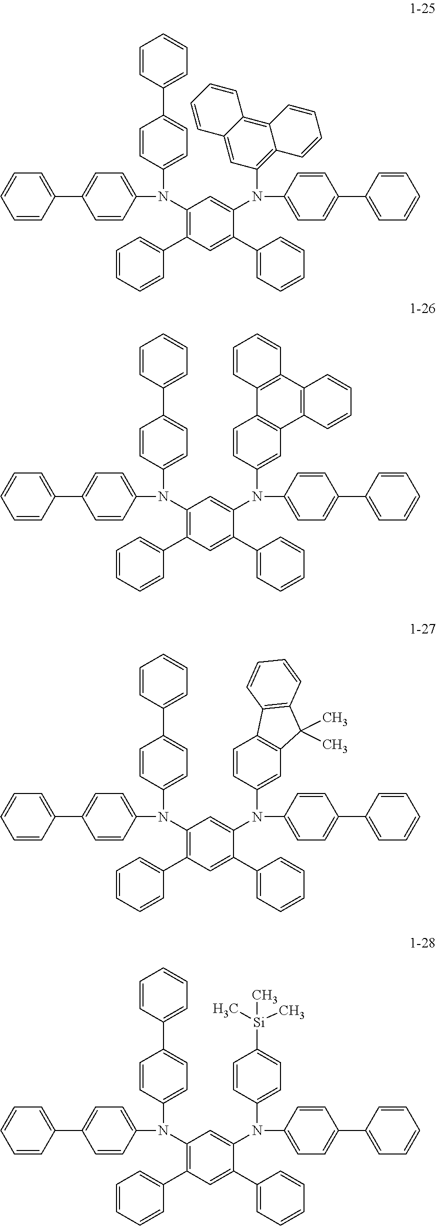 [SMILES: c1ccc(-c2ccc(N(c3ccc(-c4ccccc4)cc3)c3cc(N(c4ccc(-c5ccccc5)cc4)c4cc5ccccc5c5ccccc45)c(-c4ccccc4)cc3-c3ccccc3)cc2)cc1 ; c1ccc(-c2ccc(N(c3ccc(-c4ccccc4)cc3)c3cc(N(c4ccc(-c5ccccc5)cc4)c4ccc5c6ccccc6c6ccccc6c5c4)c(-c4ccccc4)cc3-c3ccccc3)cc2)cc1 ; CC1(C)c2ccccc2-c2ccc(N(c3ccc(-c4ccccc4)cc3)c3cc(N(c4ccc(-c5ccccc5)cc4)c4ccc(-c5ccccc5)cc4)c(-c4ccccc4)cc3-c3ccccc3)cc21 ; C[Si](C)(C)c1ccc(N(c2ccc(-c3ccccc3)cc2)c2cc(N(c3ccc(-c4ccccc4)cc3)c3ccc(-c4ccccc4)cc3)c(-c3ccccc3)cc2-c2ccccc2)cc1]

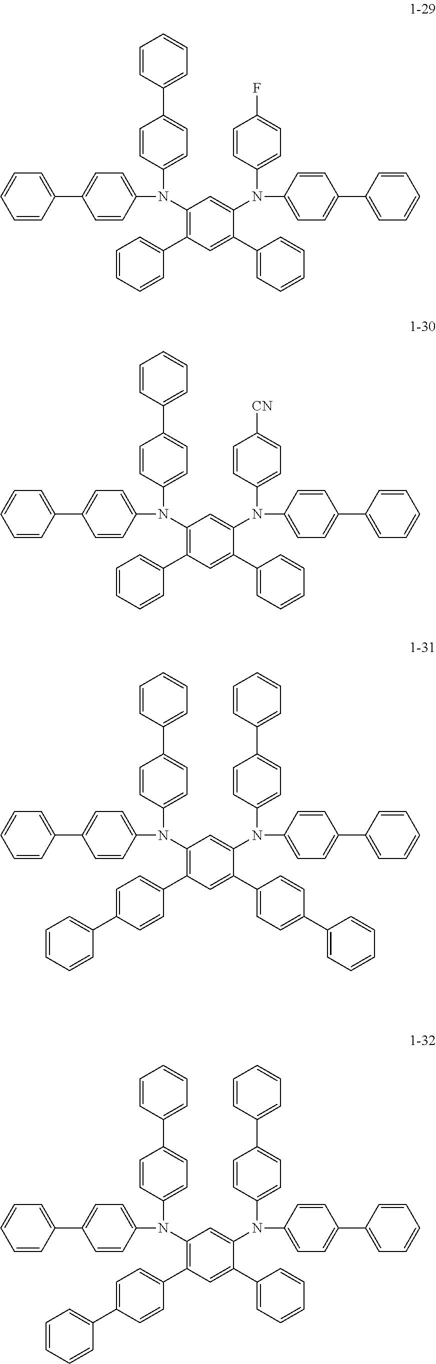 [SMILES: Fc1ccc(N(c2ccc(-c3ccccc3)cc2)c2cc(N(c3ccc(-c4ccccc4)cc3)c3ccc(-c4ccccc4)cc3)c(-c3ccccc3)cc2-c2ccccc2)cc1 ; N#Cc1ccc(N(c2ccc(-c3ccccc3)cc2)c2cc(N(c3ccc(-c4ccccc4)cc3)c3ccc(-c4ccccc4)cc3)c(-c3ccccc3)cc2-c2ccccc2)cc1 ; c1ccc(-c2ccc(-c3cc(-c4ccc(-c5ccccc5)cc4)c(N(c4ccc(-c5ccccc5)cc4)c4ccc(-c5ccccc5)cc4)cc3N(c3ccc(-c4ccccc4)cc3)c3ccc(-c4ccccc4)cc3)cc2)cc1 ; c1ccc(-c2ccc(-c3cc(-c4ccccc4)c(N(c4ccc(-c5ccccc5)cc4)c4ccc(-c5ccccc5)cc4)cc3N(c3ccc(-c4ccccc4)cc3)c3ccc(-c4ccccc4)cc3)cc2)cc1]

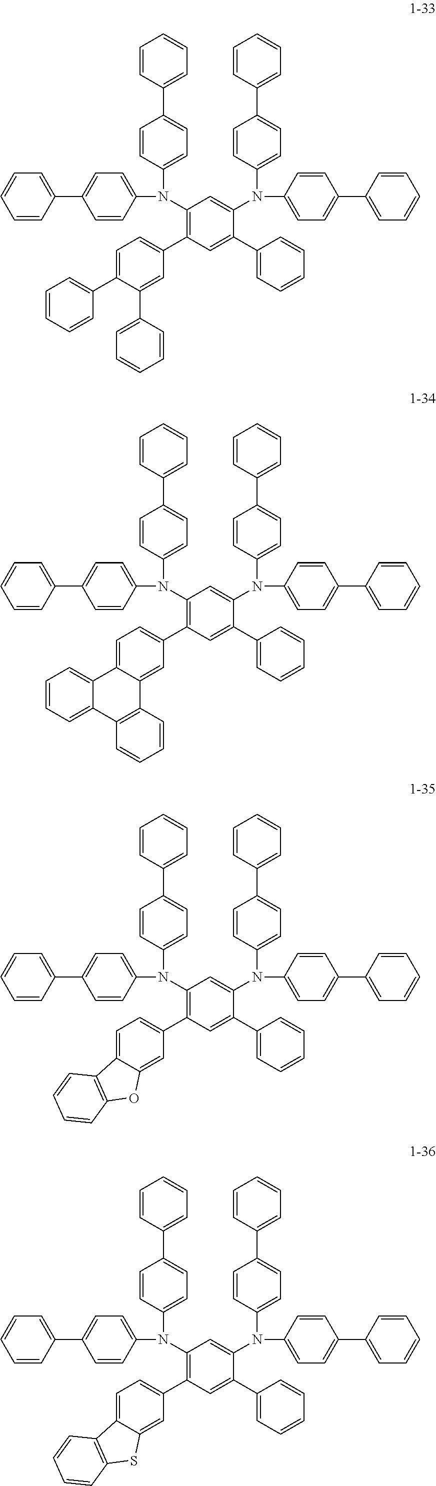

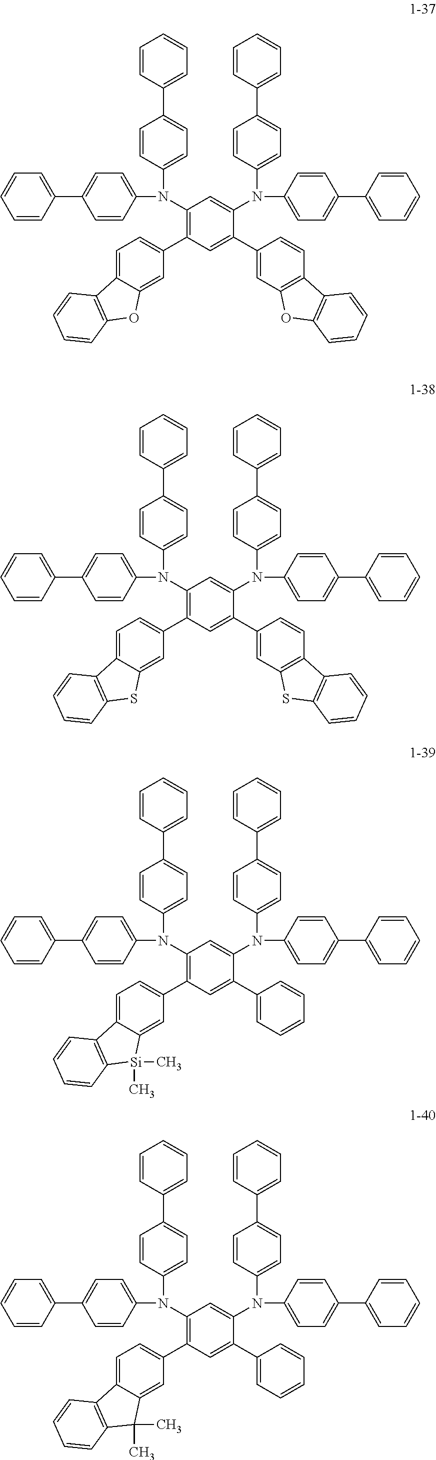

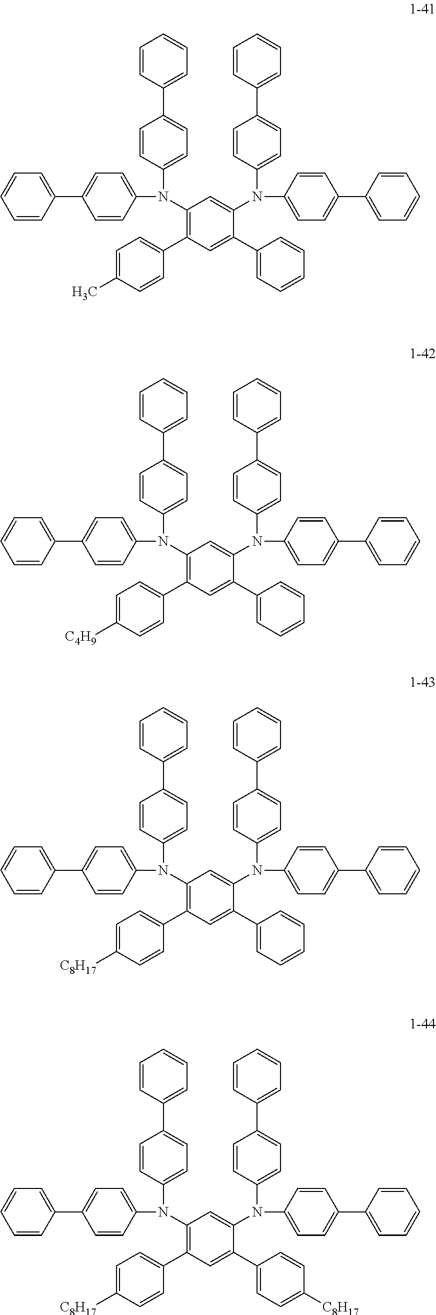

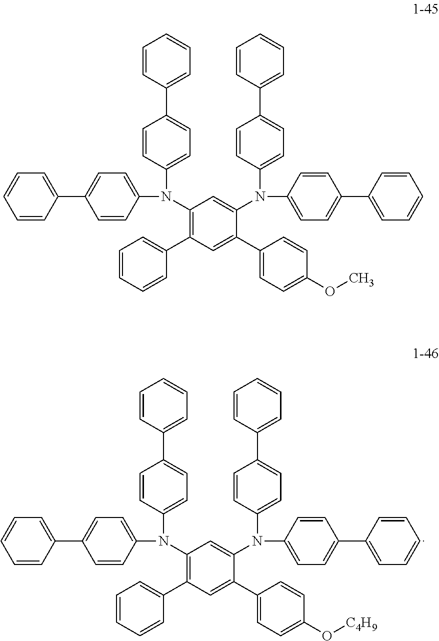

The compound may include one of the following Compounds 1-47 to 1-58:

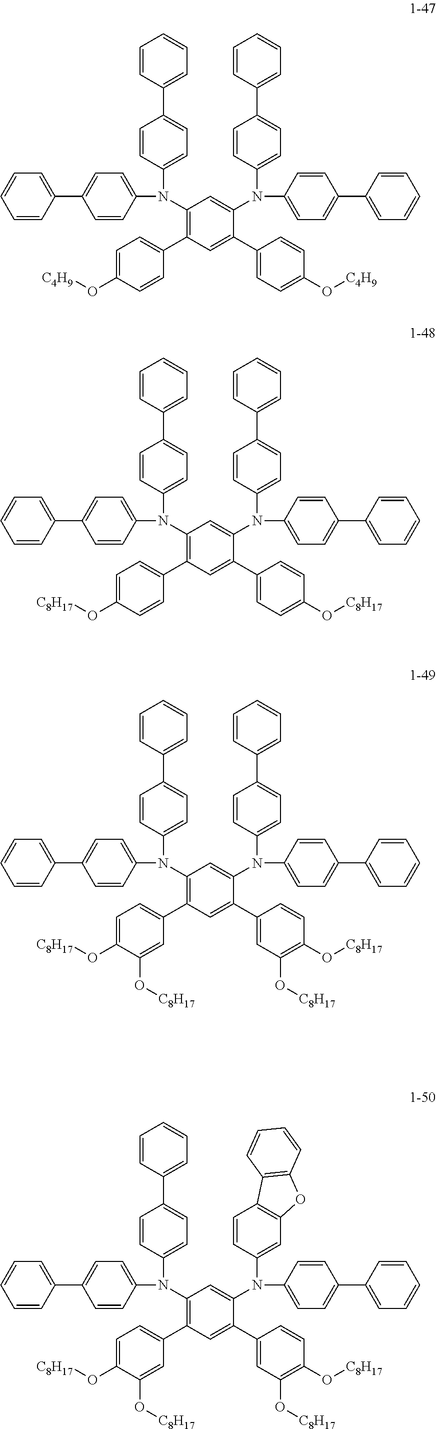

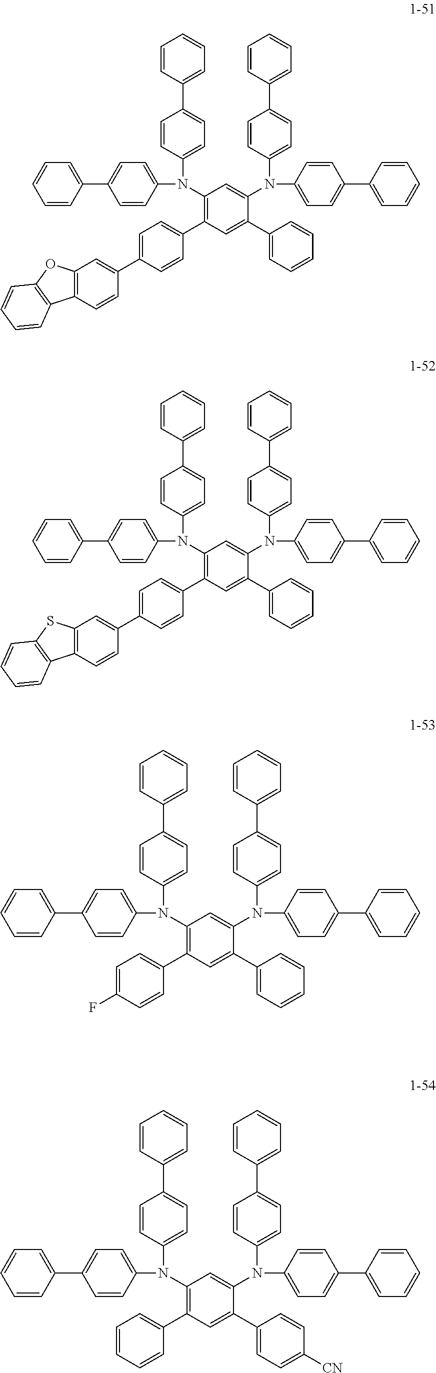

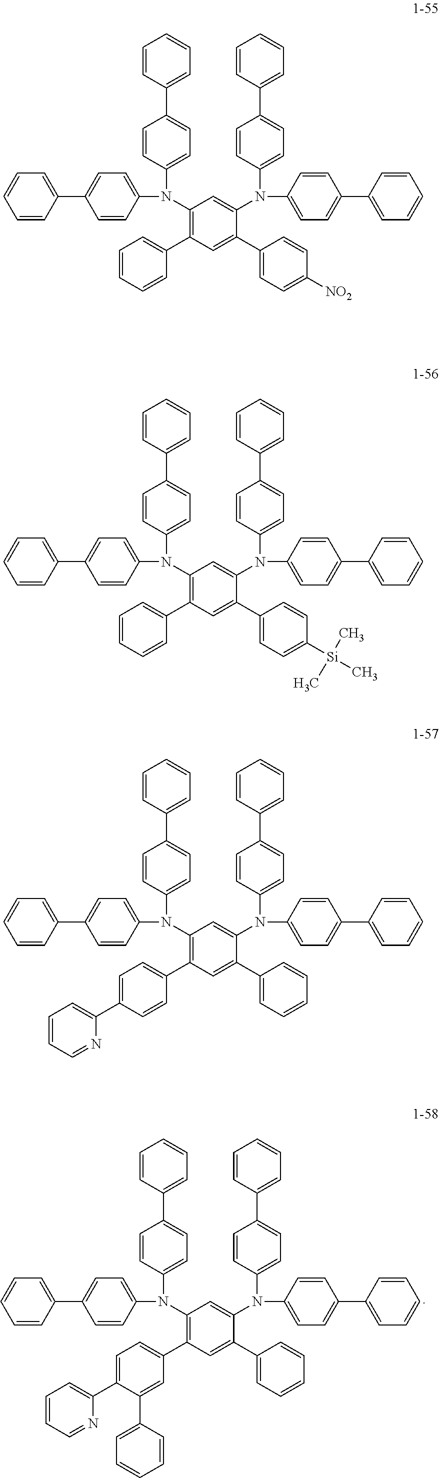

The organic electroluminescent device may include a hole transport layer, and the compound for an organic electroluminescent device may be included in the hole transport layer.

### BRIEF DESCRIPTION OF THE DRAWING

Features will be apparent to those of skill in the art by describing in detail exemplary embodiments with reference to the attached drawing in which:

FIG. 1 illustrates a cross-sectional view of an organic electroluminescent device according to an embodiment.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. Like reference numerals refer to like elements throughout.

<1. Constitution of Material for Organic Electroluminescent Device>

A material or compound for an organic electroluminescent device according to an embodiment may help improve the driving voltage, the emission efficiency, and the emission life of an organic electroluminescent device. The compound for an organic electroluminescent device may contribute to the improvement of the driving voltage, the emission efficiency and the emission life of the organic electroluminescent device, e.g., when used as a hole transport material. Here, the constitution of the compound for an organic electroluminescent device according to an embodiment will be explained. The material for an organic electroluminescent device may include a compound for an organic electroluminescent device represented by the following Formula 1.

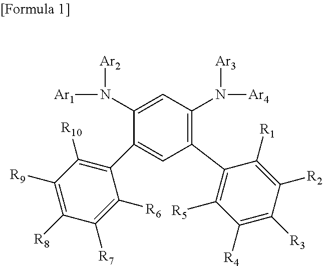

In Formula 1, Ar₁ to Ar₄ may each independently be or include, e.g., a substituted or unsubstituted aryl group or a substituted or unsubstituted heteroaryl group.

Examples of the aryl group and the heteroaryl group may include a phenyl group, a biphenyl group, a terphenyl group, a naphthyl group, an anthryl group, a phenanthryl group, a fluorenyl group, an indenyl group, a pyrenyl group, a fluoranthenyl group, a triphenylenyl group, a pyridyl group, a furanyl group, a pyranyl group, a thienyl group, a quinolyl group, an isoquinolyl group, a benzofuranyl group, a benzothienyl group, an indolyl group, a benzoxazolyl group, a benzothiazolyl group, a quinoxalyl group, a pyrazolyl group, a dibenzoheterole group, etc. In an implementation, Ar₁ to Ar₄ may each independently be, e.g., a phenyl group, a biphenyl group, a naphthyl group, a dibenzoheterole group, a phenanthryl group, or a triphenylenyl group. In an implementation, the dibenzoheterole group may be represented by the following Formula 2. In Formula 2, X may be one of O, S, or Si(R₁₁)₂. R₁₁ may be, e.g., a hydrogen, an alkyl group (for example, a methyl group, an ethyl group, etc. where the alkyl group may be substituted or unsubstituted) or an aryl group (for example, a phenyl group, etc. where the aryl group may be substituted or unsubstituted).

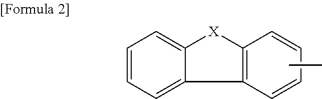

At least one of Ar₁ to Ar₄ may be or may include, e.g., a substituted or unsubstituted aryl group having at least 10 ring carbon atoms or a substituted or unsubstituted heteroaryl group having at least 10 ring carbon atoms. Examples of the aryl group and the heteroaryl group having at least 10 ring carbon atoms may include the aryl group and heteroaryl group having at least 10 ring carbon atoms among the above-illustrated examples of the aryl group and heteroaryl group. In an implementation, Ar₁ to Ar₄ may be, e.g., a biphenyl group, a naphthyl group, a dibenzoheterole group, a phenanthryl group, or a triphenylenyl group. In an implementation, Ar₁ to Ar₄ may be, e.g., a biphenyl group or a dibenzoheterole group. In an implementation, all of Ar₁ to Ar₄ may be or include, e.g., the substituted or unsubstituted aryl group having at least 10 ring carbon atoms or the substituted or unsubstituted heteroaryl group having at least 10 ring carbon atoms ring. In this case, the stability of cationic radicals may be improved.

In an implementation, a substituent of the substituted aryl group or the substituted heteroaryl group of Ar₁ to Ar₄ may include, e.g., an alkyl group (for example, a methyl group, an ethyl group, etc.), a halogen atom (for example, a fluorine atom, a chlorine atom, etc.), a silyl group (for example, a trimethylsilyl group), a cyano group, an alkoxy group (for example, a methoxy group, a butoxy group, an octoxy group, etc.), a nitro group, a hydroxyl group, a thiol group, etc.

R₁ to R₁₀ may each independently be or include, e.g., a hydrogen atom, a substituted or unsubstituted aryl group, a substituted or unsubstituted heteroaryl group, a substituted or unsubstituted alkyl group (for example, a methyl group, an ethyl group, etc.), a substituted or unsubstituted alkoxy group (for example, a methoxy group, a butoxy group and a dioctoxy group), a halogen atom (for example, a fluorine atom, a chlorine atom, etc.), an electron withdrawing group (for example, a nitro group, etc.) or a deuterium atom. In an implementation, R₁ to R₁₀ may be separate or adjacent ones thereof may be combined or bound to each other to form a saturated or unsaturated ring.

Examples of the aryl group and the heteroaryl group of R₁ to R₁₀ may be the same as those of the aryl group and the heteroaryl group of Ar₁ to Ar₄. In an implementation, a substituent of the substituted aryl group, the substituted heteroaryl group, the substituted alkyl group, or the substituted alkoxy group of R₁ to R₁₀ may include, e.g., an alkyl group (for example, a methyl group), a halogen atom (for example, a fluorine atom), a silyl group (for example, a trimethylsilyl group), a cyano group, an alkoxy group (for example, a methoxy group, a butoxy group, an octoxy group), a nitro group, a hydroxyl group, a thiol group, etc.

The compound for an organic electroluminescent device according to an embodiment may be included in a material of at least one of a hole transport layer or an emission layer, which are layers for forming the organic electroluminescent device. In an implementation, the compound for an organic electroluminescent device may be included in the hole transport layer.

An organic electroluminescent device including the compound for an organic electroluminescent device according to an embodiment may have largely improved driving voltage, emission efficiency, and emission life as described below.

The compound for an organic electroluminescent device according to an embodiment may have a low HOMO level. For example, as shown in the following embodiments, the HOMO level of the compound for an organic electroluminescent device according to an embodiment may be less than or equal to about −5.7 eV. The HOMO level of another hole transport material may be about −5.3 eV. Thus, by including the compound for an organic electroluminescent device according to an embodiment in the hole transport layer, holes may be easily injected from a hole injection layer to the hole transport layer.

In the compound for an organic electroluminescent device according to an embodiment, at least one of Ar₁ to Ar₄ may be the aryl group or the heteroaryl group having at least 10 ring carbon atoms, and the molecular weight of the compound may be relatively large. Thus, the glass transition temperature of the compound may be relatively high. Accordingly, the thermal stability of the compound may be increased, and a layer quality may be improved, thereby realizing the long life or the high efficiency of a device. In an implementation, the compound for an organic electroluminescent device according to an embodiment may be, e.g., one of the following Compounds 1-1 to 1-58.

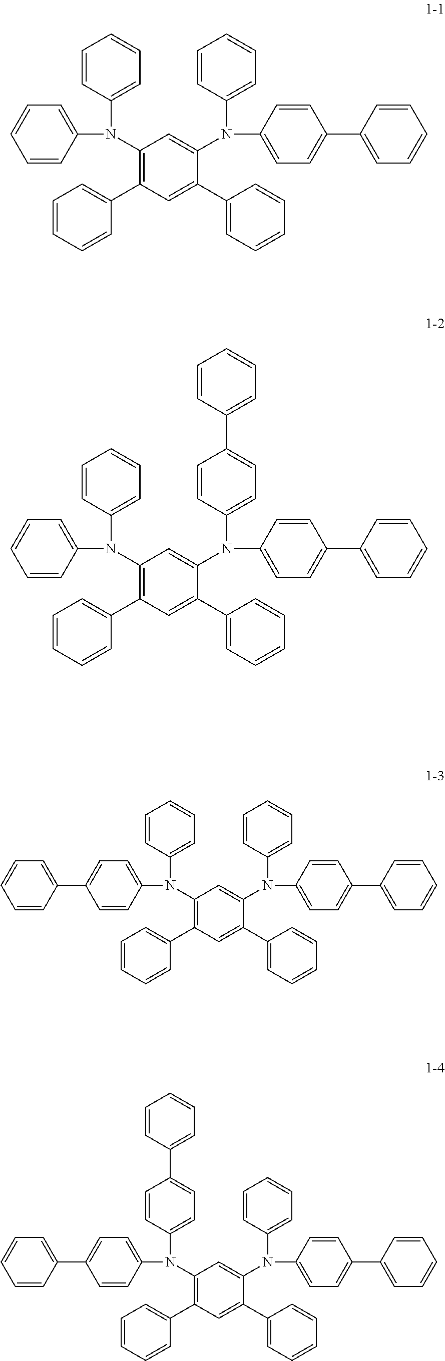 [SMILES: c1ccc(-c2ccc(N(c3ccccc3)c3cc(N(c4ccccc4)c4ccccc4)c(-c4ccccc4)cc3-c3ccccc3)cc2)cc1 ; c1ccc(-c2ccc(N(c3ccc(-c4ccccc4)cc3)c3cc(N(c4ccccc4)c4ccccc4)c(-c4ccccc4)cc3-c3ccccc3)cc2)cc1 ; c1ccc(-c2ccc(N(c3ccccc3)c3cc(N(c4ccccc4)c4ccc(-c5ccccc5)cc4)c(-c4ccccc4)cc3-c3ccccc3)cc2)cc1 ; c1ccc(-c2ccc(N(c3ccccc3)c3cc(N(c4ccc(-c5ccccc5)cc4)c4ccc(-c5ccccc5)cc4)c(-c4ccccc4)cc3-c3ccccc3)cc2)cc1]

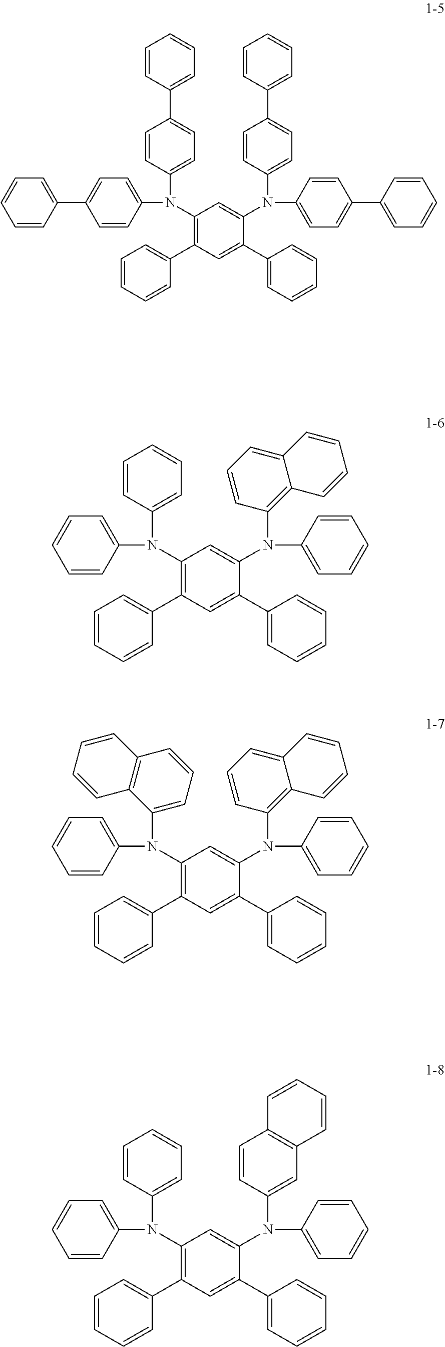 [SMILES: c1ccc(-c2ccc(N(c3ccc(-c4ccccc4)cc3)c3cc(N(c4ccc(-c5ccccc5)cc4)c4ccc(-c5ccccc5)cc4)c(-c4ccccc4)cc3-c3ccccc3)cc2)cc1 ; c1ccc(-c2cc(-c3ccccc3)c(N(c3ccccc3)c3cccc4ccccc34)cc2N(c2ccccc2)c2ccccc2)cc1 ; c1ccc(-c2cc(-c3ccccc3)c(N(c3ccccc3)c3cccc4ccccc34)cc2N(c2ccccc2)c2cccc3ccccc23)cc1 ; c1ccc(-c2cc(-c3ccccc3)c(N(c3ccccc3)c3ccc4ccccc4c3)cc2N(c2ccccc2)c2ccccc2)cc1]

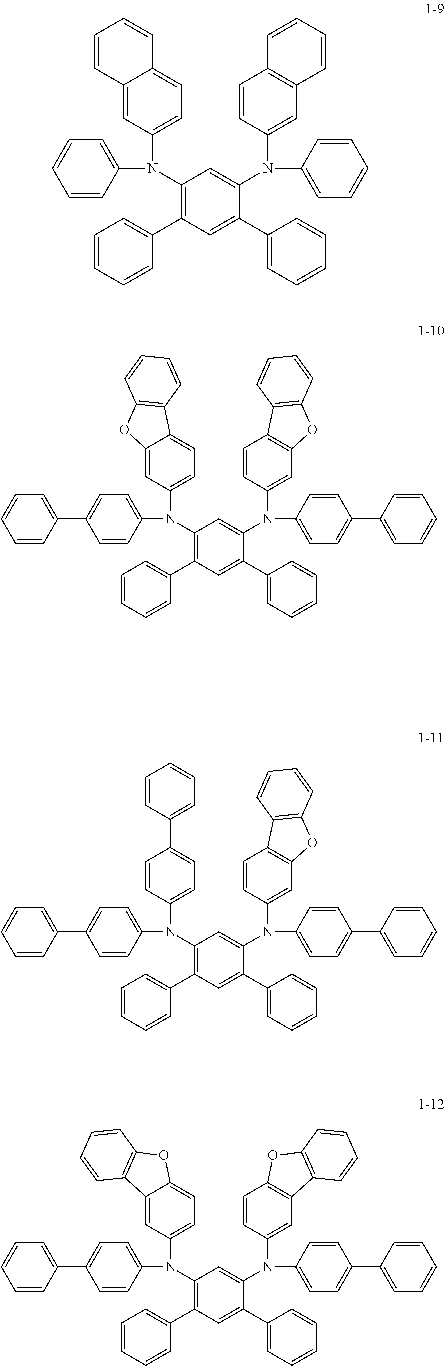 [SMILES: c1ccc(-c2cc(-c3ccccc3)c(N(c3ccccc3)c3ccc4ccccc4c3)cc2N(c2ccccc2)c2ccc3ccccc3c2)cc1 ; c1ccc(-c2ccc(N(c3ccc4c(c3)oc3ccccc34)c3cc(N(c4ccc(-c5ccccc5)cc4)c4ccc5c(c4)oc4ccccc45)c(-c4ccccc4)cc3-c3ccccc3)cc2)cc1 ; c1ccc(-c2ccc(N(c3ccc(-c4ccccc4)cc3)c3cc(N(c4ccc(-c5ccccc5)cc4)c4ccc5c(c4)oc4ccccc45)c(-c4ccccc4)cc3-c3ccccc3)cc2)cc1 ; c1ccc(-c2ccc(N(c3ccc4oc5ccccc5c4c3)c3cc(N(c4ccc(-c5ccccc5)cc4)c4ccc5oc6ccccc6c5c4)c(-c4ccccc4)cc3-c3ccccc3)cc2)cc1]

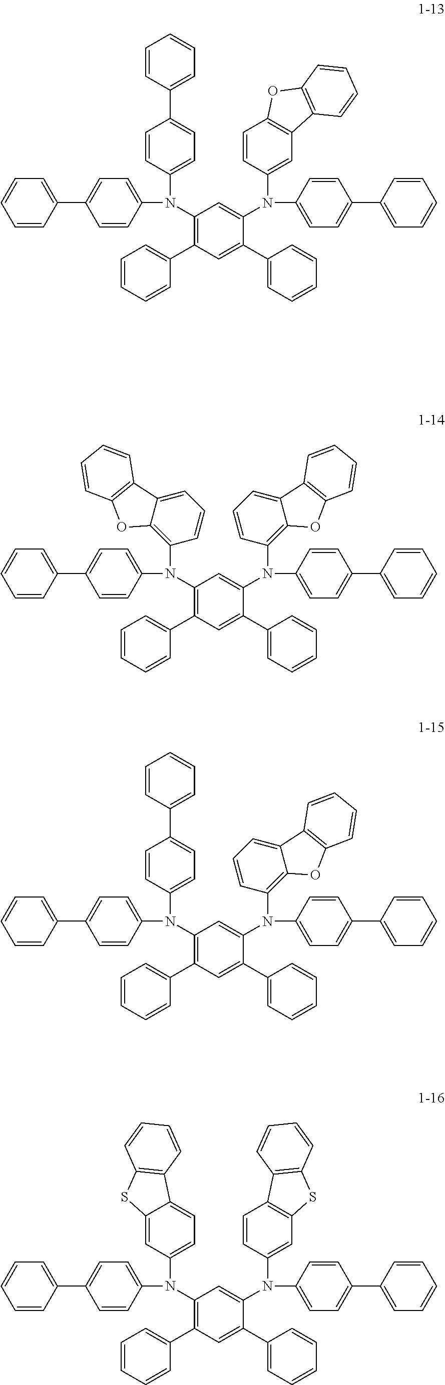 [SMILES: c1ccc(-c2ccc(N(c3ccc(-c4ccccc4)cc3)c3cc(N(c4ccc(-c5ccccc5)cc4)c4ccc5oc6ccccc6c5c4)c(-c4ccccc4)cc3-c3ccccc3)cc2)cc1 ; c1ccc(-c2ccc(N(c3cc(N(c4ccc(-c5ccccc5)cc4)c4cccc5c4oc4ccccc45)c(-c4ccccc4)cc3-c3ccccc3)c3cccc4c3oc3ccccc34)cc2)cc1 ; c1ccc(-c2ccc(N(c3ccc(-c4ccccc4)cc3)c3cc(N(c4ccc(-c5ccccc5)cc4)c4cccc5c4oc4ccccc45)c(-c4ccccc4)cc3-c3ccccc3)cc2)cc1 ; c1ccc(-c2ccc(N(c3ccc4c(c3)sc3ccccc34)c3cc(N(c4ccc(-c5ccccc5)cc4)c4ccc5c(c4)sc4ccccc45)c(-c4ccccc4)cc3-c3ccccc3)cc2)cc1]

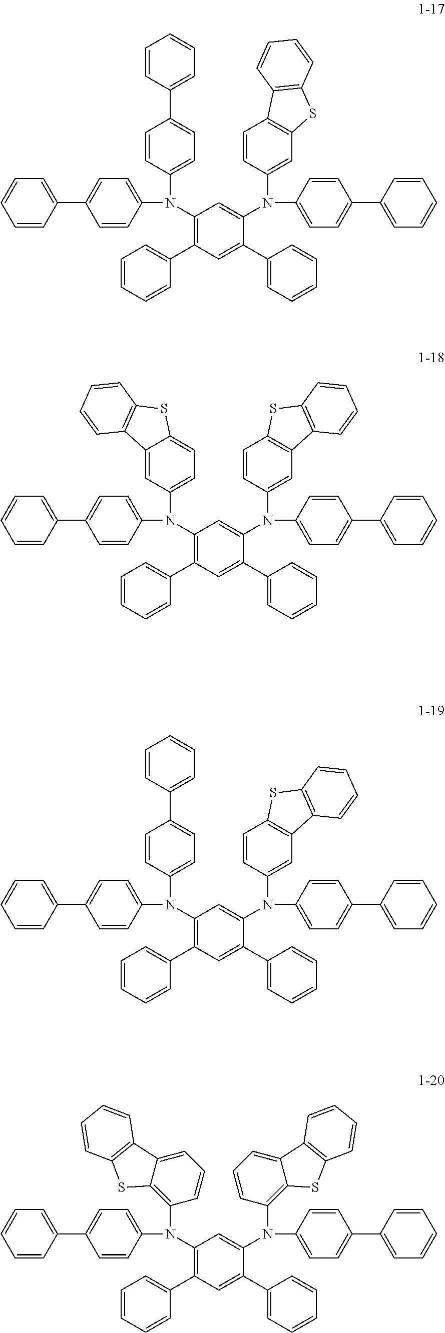 [SMILES: c1ccc(-c2ccc(N(c3ccc(-c4ccccc4)cc3)c3cc(N(c4ccc(-c5ccccc5)cc4)c4ccc5c(c4)sc4ccccc45)c(-c4ccccc4)cc3-c3ccccc3)cc2)cc1 ; c1ccc(-c2ccc(N(c3ccc4sc5ccccc5c4c3)c3cc(N(c4ccc(-c5ccccc5)cc4)c4ccc5sc6ccccc6c5c4)c(-c4ccccc4)cc3-c3ccccc3)cc2)cc1 ; c1ccc(-c2ccc(N(c3ccc(-c4ccccc4)cc3)c3cc(N(c4ccc(-c5ccccc5)cc4)c4ccc5sc6ccccc6c5c4)c(-c4ccccc4)cc3-c3ccccc3)cc2)cc1 ; c1ccc(-c2ccc(N(c3cc(N(c4ccc(-c5ccccc5)cc4)c4cccc5c4sc4ccccc45)c(-c4ccccc4)cc3-c3ccccc3)c3cccc4c3sc3ccccc34)cc2)cc1]

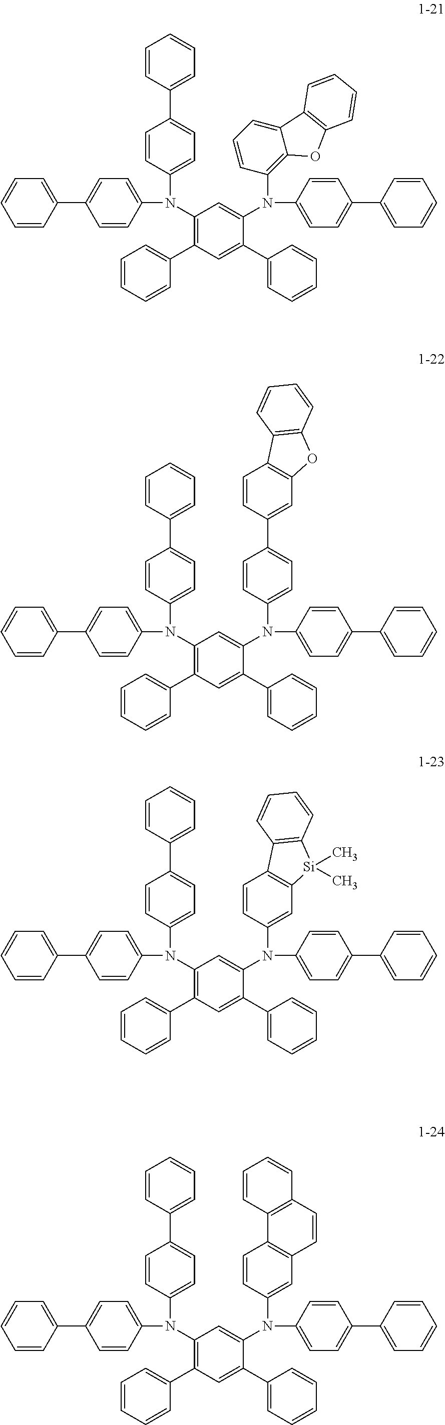 [SMILES: c1ccc(-c2ccc(N(c3ccc(-c4ccccc4)cc3)c3cc(N(c4ccc(-c5ccccc5)cc4)c4cccc5c4oc4ccccc45)c(-c4ccccc4)cc3-c3ccccc3)cc2)cc1 ; c1ccc(-c2ccc(N(c3ccc(-c4ccccc4)cc3)c3cc(N(c4ccc(-c5ccccc5)cc4)c4ccc(-c5ccc6c(c5)oc5ccccc56)cc4)c(-c4ccccc4)cc3-c3ccccc3)cc2)cc1 ; C[Si]1(C)c2ccccc2-c2ccc(N(c3ccc(-c4ccccc4)cc3)c3cc(N(c4ccc(-c5ccccc5)cc4)c4ccc(-c5ccccc5)cc4)c(-c4ccccc4)cc3-c3ccccc3)cc21 ; c1ccc(-c2ccc(N(c3ccc(-c4ccccc4)cc3)c3cc(N(c4ccc(-c5ccccc5)cc4)c4ccc5c(ccc6ccccc65)c4)c(-c4ccccc4)cc3-c3ccccc3)cc2)cc1]

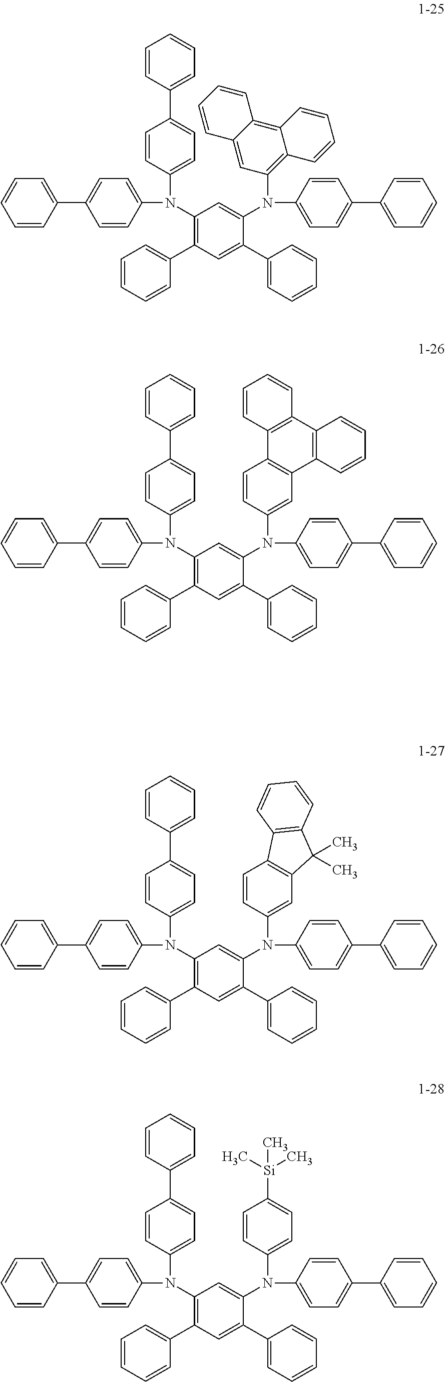 [SMILES: c1ccc(-c2ccc(N(c3ccc(-c4ccccc4)cc3)c3cc(N(c4ccc(-c5ccccc5)cc4)c4cc5ccccc5c5ccccc45)c(-c4ccccc4)cc3-c3ccccc3)cc2)cc1 ; c1ccc(-c2ccc(N(c3ccc(-c4ccccc4)cc3)c3cc(N(c4ccc(-c5ccccc5)cc4)c4ccc5c6ccccc6c6ccccc6c5c4)c(-c4ccccc4)cc3-c3ccccc3)cc2)cc1 ; CC1(C)c2ccccc2-c2ccc(N(c3ccc(-c4ccccc4)cc3)c3cc(N(c4ccc(-c5ccccc5)cc4)c4ccc(-c5ccccc5)cc4)c(-c4ccccc4)cc3-c3ccccc3)cc21 ; C[Si](C)(C)c1ccc(N(c2ccc(-c3ccccc3)cc2)c2cc(N(c3ccc(-c4ccccc4)cc3)c3ccc(-c4ccccc4)cc3)c(-c3ccccc3)cc2-c2ccccc2)cc1]

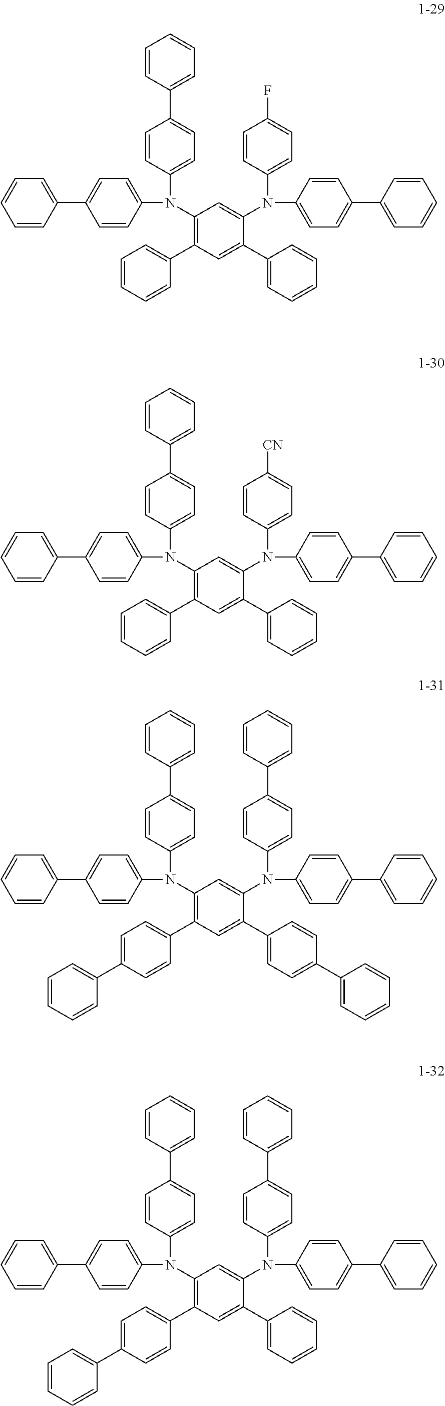 [SMILES: Fc1ccc(N(c2ccc(-c3ccccc3)cc2)c2cc(N(c3ccc(-c4ccccc4)cc3)c3ccc(-c4ccccc4)cc3)c(-c3ccccc3)cc2-c2ccccc2)cc1 ; N#Cc1ccc(N(c2ccc(-c3ccccc3)cc2)c2cc(N(c3ccc(-c4ccccc4)cc3)c3ccc(-c4ccccc4)cc3)c(-c3ccccc3)cc2-c2ccccc2)cc1 ; c1ccc(-c2ccc(-c3cc(-c4ccc(-c5ccccc5)cc4)c(N(c4ccc(-c5ccccc5)cc4)c4ccc(-c5ccccc5)cc4)cc3N(c3ccc(-c4ccccc4)cc3)c3ccc(-c4ccccc4)cc3)cc2)cc1 ; c1ccc(-c2ccc(-c3cc(-c4ccccc4)c(N(c4ccc(-c5ccccc5)cc4)c4ccc(-c5ccccc5)cc4)cc3N(c3ccc(-c4ccccc4)cc3)c3ccc(-c4ccccc4)cc3)cc2)cc1]

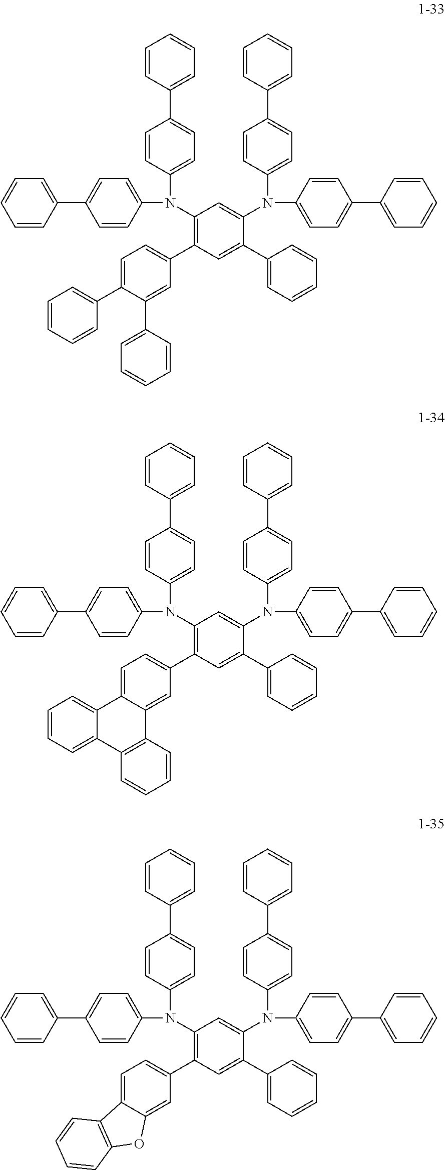 [SMILES: c1ccc(-c2ccc(N(c3ccc(-c4ccccc4)cc3)c3cc(N(c4ccc(-c5ccccc5)cc4)c4ccc(-c5ccccc5)cc4)c(-c4ccc(-c5ccccc5)c(-c5ccccc5)c4)cc3-c3ccccc3)cc2)cc1 ; c1ccc(-c2ccc(N(c3ccc(-c4ccccc4)cc3)c3cc(N(c4ccc(-c5ccccc5)cc4)c4ccc(-c5ccccc5)cc4)c(-c4ccc5c6ccccc6c6ccccc6c5c4)cc3-c3ccccc3)cc2)cc1 ; c1ccc(-c2ccc(N(c3ccc(-c4ccccc4)cc3)c3cc(N(c4ccc(-c5ccccc5)cc4)c4ccc(-c5ccccc5)cc4)c(-c4ccc5c(c4)oc4ccccc45)cc3-c3ccccc3)cc2)cc1]

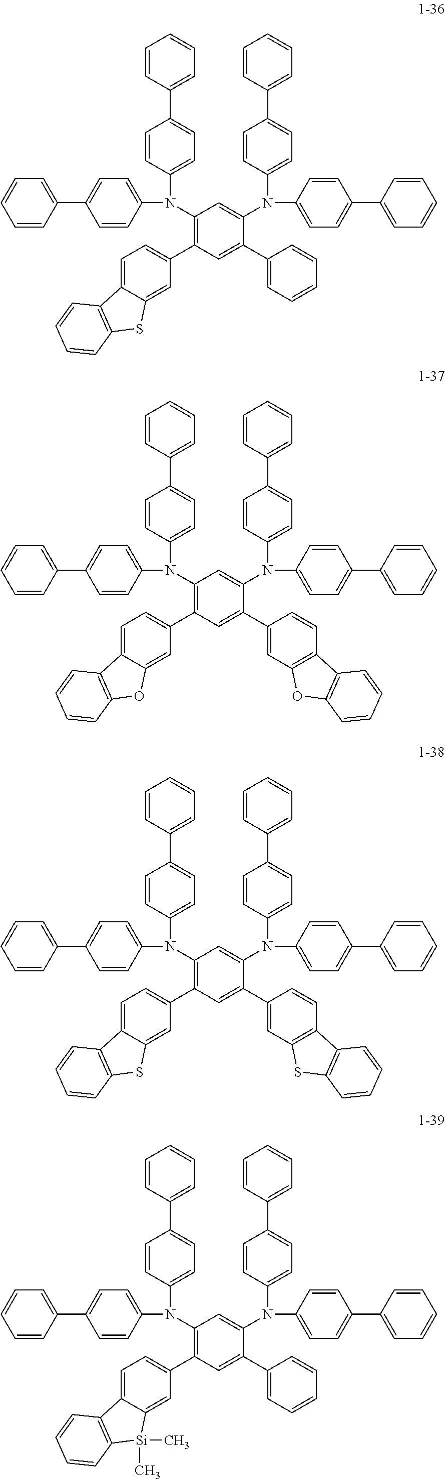

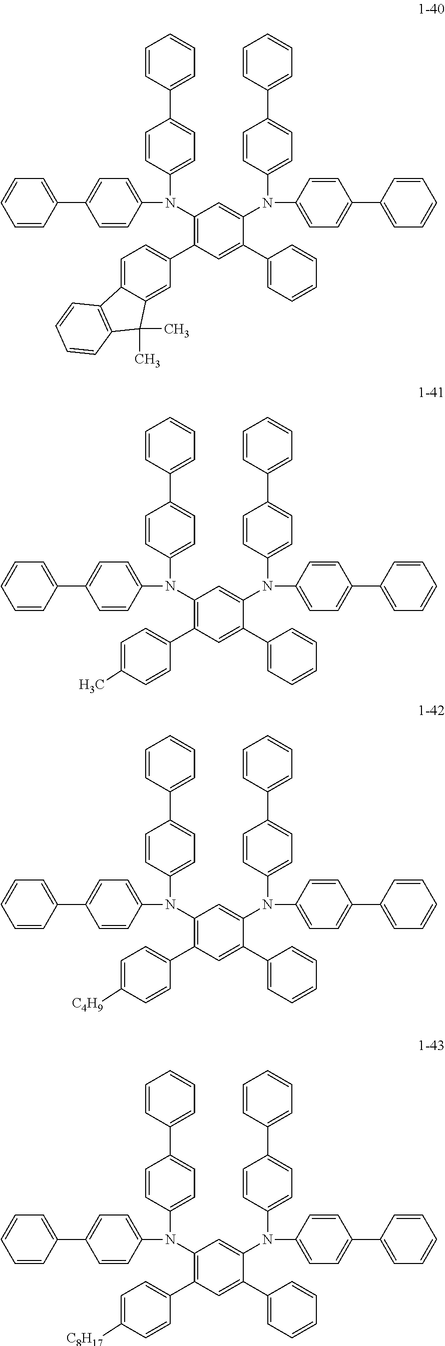

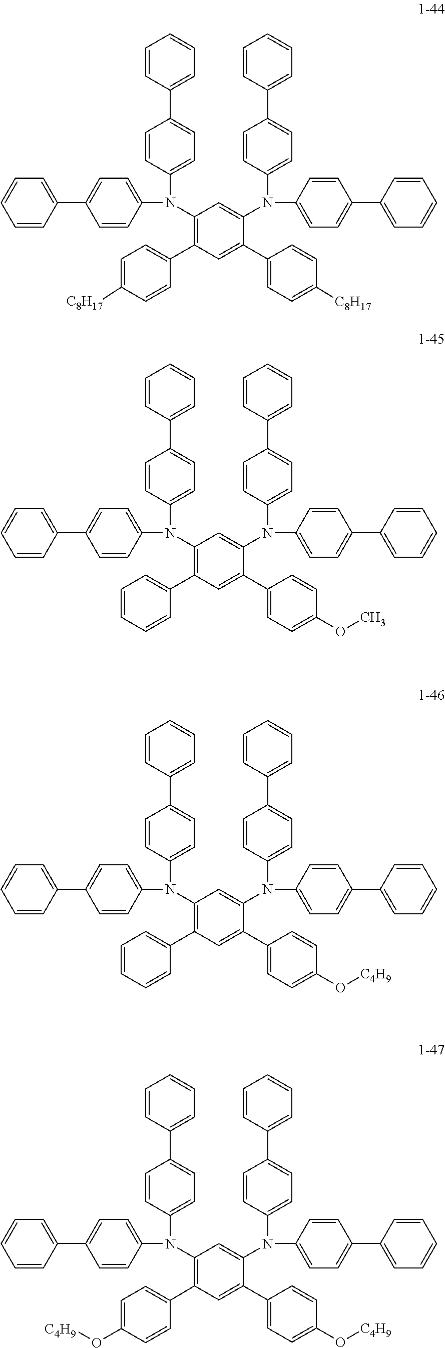

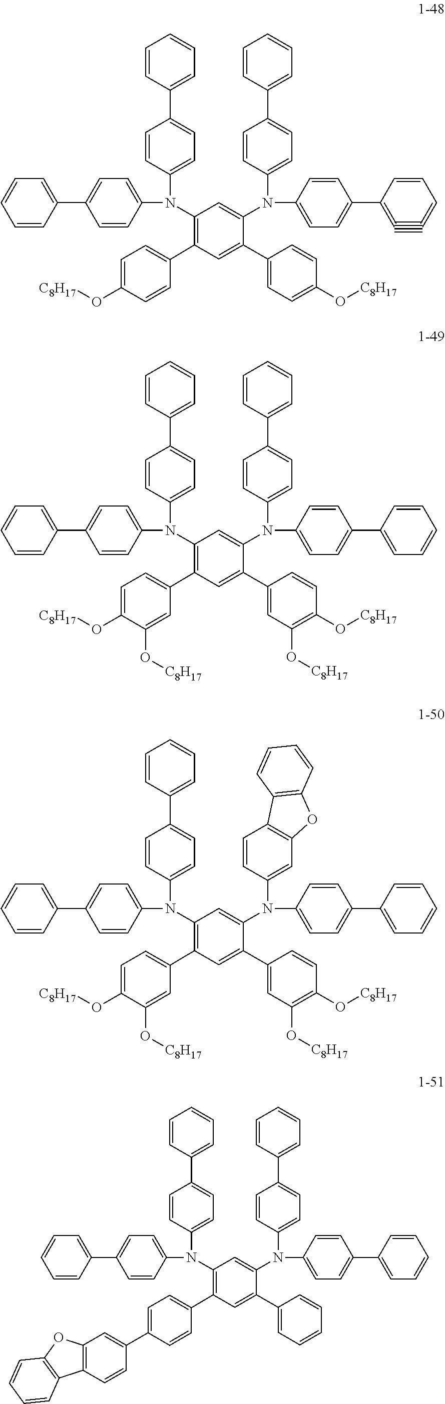

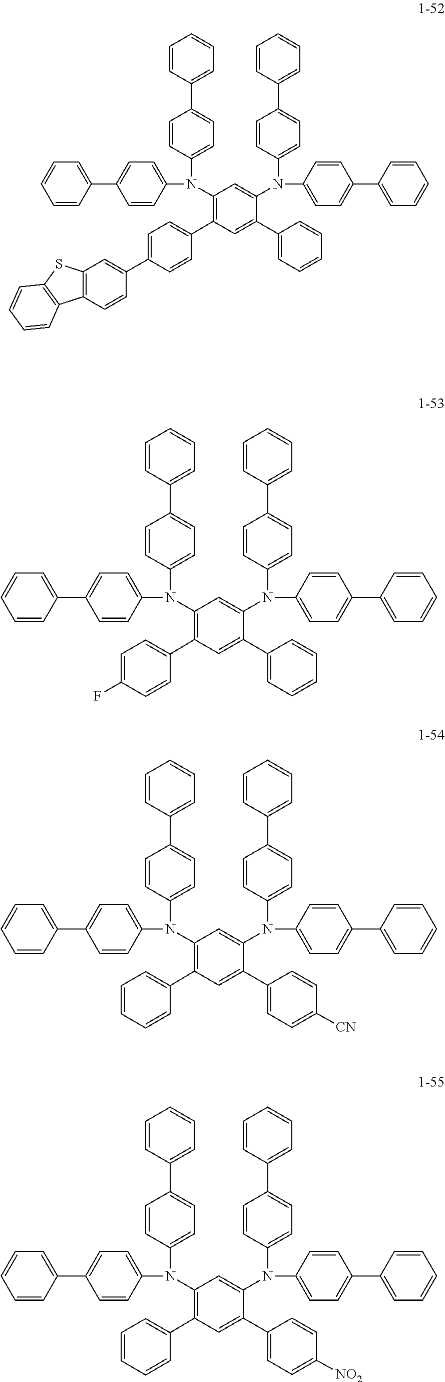

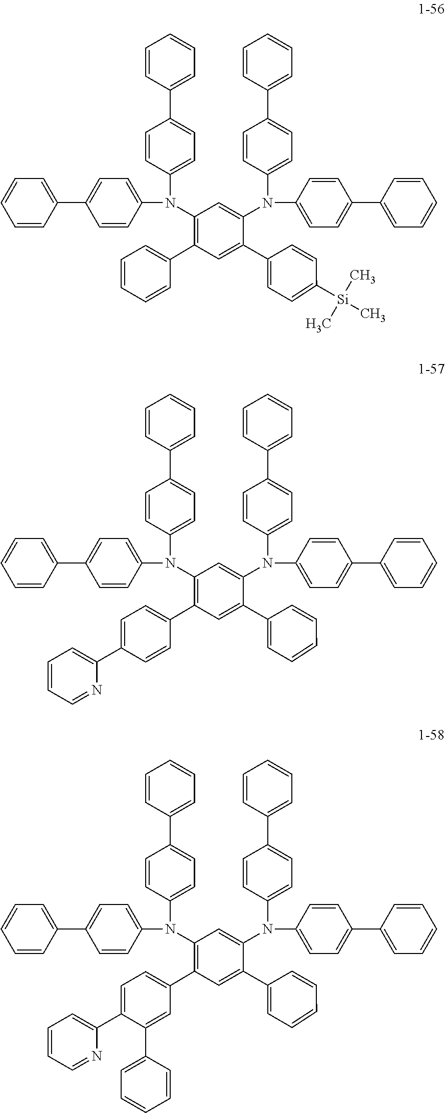 [SMILES: C[Si](C)(C)c1ccc(-c2cc(-c3ccccc3)c(N(c3ccc(-c4ccccc4)cc3)c3ccc(-c4ccccc4)cc3)cc2N(c2ccc(-c3ccccc3)cc2)c2ccc(-c3ccccc3)cc2)cc1 ; c1ccc(-c2ccc(N(c3ccc(-c4ccccc4)cc3)c3cc(N(c4ccc(-c5ccccc5)cc4)c4ccc(-c5ccccc5)cc4)c(-c4ccc(-c5ccccn5)cc4)cc3-c3ccccc3)cc2)cc1 ; c1ccc(-c2ccc(N(c3ccc(-c4ccccc4)cc3)c3cc(N(c4ccc(-c5ccccc5)cc4)c4ccc(-c5ccccc5)cc4)c(-c4ccc(-c5ccccn5)c(-c5ccccc5)c4)cc3-c3ccccc3)cc2)cc1]

<2. Organic Electroluminescent Device Including the Compound for Organic Electroluminescent Device>

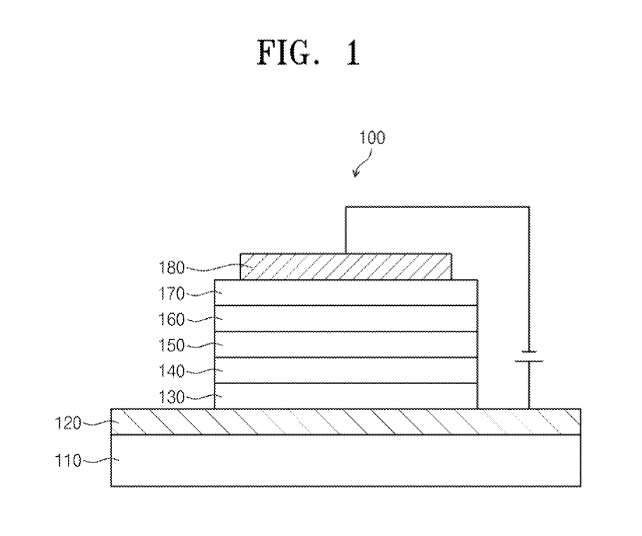

Hereinafter, referring to FIG. 1, an organic electroluminescent device using a material for an organic electroluminescent device according to an embodiment will be explained in brief. FIG. 1 illustrates a schematic cross-sectional view of an organic electroluminescent device according to an embodiment.

As shown in FIG. 1, an organic EL device 100 according to an embodiment may include a substrate 110, a first electrode 120 disposed on the substrate 110, a hole injection layer 130 disposed on the first electrode 120, a hole transport layer 140 disposed on the hole injection layer 130, an emission layer 150 disposed on the hole transport layer 140, an electron transport layer 160 disposed on the emission layer 150, an electron injection layer 170 disposed on the electron transport layer 160, and a second electrode 180 disposed on the electron injection layer 170.

The compound for an organic electroluminescent device according to an embodiment may be included in at least one layer of the hole transport layer and the emission layer. In an implementation, the compound for organic electroluminescent device may be included in both the hole transport layer and the emission layer. In an implementation, the compound for an organic electroluminescent device may be included in the hole transport layer 140.

Each organic thin layer disposed between the first electrode 120 and the second electrode 180 of the organic electroluminescent device may be formed by various methods, e.g., an evaporation method.

The substrate 110 may be a substrate commonly used in an organic electroluminescent device. For example, the substrate 110 may be a glass substrate, a semiconductor substrate, a transparent plastic substrate, etc.

The first electrode 120 may be formed on the substrate 110 by an evaporation method or a sputtering method. For example, the first electrode 120 may be formed as a transmission type electrode using a metal having high work function, an alloy, a conductive compound, etc. The first electrode 120 may be formed using, e.g., a transparent and highly conductive indium tin oxide (ITO), indium zinc oxide (IZO), tin oxide (SnO₂), zinc oxide (ZnO), etc. In an implementation, an anode 120 may be formed as a reflection type electrode using magnesium (Mg), aluminum (Al), etc.

The hole injection layer 130 may be a layer provided with the function of easy injection of holes from the first electrode 120 and may be formed on the first electrode 120 to a thickness of about 10 nm to about 150 nm. The hole injection layer 130 may be formed using a suitable material. The material may include, e.g., triphenylamine-containing poly ether ketone (TPAPEK), 4-isopropyl-4-methyldiphenyliodoniumtetrakis(pentafluorophenyl)borate (PPBI), N,N′-diphenyl-N,N-bis-[4-(phenyl-m-tolyl-amino)-phenyl]-biphenyl-4,4-diamine (DNTPD), a phthalocyanine compound such as copper phthalocyanine, 4,4,4″-tris(3-methylphenylamino)triphenylamine (m-MTDATA), N,N-di(1-naphthyl)-N,N-diphenylbenzidine (NPB), 4,4,4″-tris{N,N-diamino}triphenylamine (TDATA), 4,4,4″-tris(N,N-2-naphthylamino)triphenylamine (2-TNATA), polyaniline/dodecylbenzenesulfonic acid (Pani/DBSA), poly(3,4-ethylenedioxythiophene)/poly(4-styrenesulfonate)/ (PEDOT/PSS), polyaniline/(camphor)sulfonic acid (Pani/CSA), polyaniline/poly(4-styrenesulfonate (PANI/PSS), etc.

The hole transport layer 140 may be a layer formed using a hole transport material provided with the function of transporting holes and may be formed, e.g., on the hole injection layer 130 to a thickness from about 10 nm to about 150 nm. The hole transport layer 140 may be formed using the compound for an organic electroluminescent device according to an embodiment. In an implementation, when the compound for an organic electroluminescent device according to an embodiment is used as a host material of the emission layer 150, the hole transport layer 140 may be formed using a suitable hole transport material. The suitable hole transport material may include, e.g., a carbazole derivative such as 1,1-bis[(di-4-tolylamino)phenyl]cyclohexane (TAPC), N-phenyl carbazole, polyvinyl carbazole, etc., N,N-bis(3-methylphenyl)-N,N-diphenyl-[1,1-biphenyl]-4,4-diamine (TPD), 4,4,4″-tris(N-carbazolyl)triphenylamine (TCTA), N,N-di(1-naphthyl)-N,N-diphenylbenzidine (NPB), etc.

The emission layer 150 may be a layer emitting light via fluorescence or phosphorescence. The emission layer 150 may be formed by including a host material and a luminescent dopant material. In an implementation, the emission layer 150 may particularly be formed to a thickness from about 10 nm to about 60 nm.

The compound for an organic electroluminescent device according to an embodiment may be used as the host of the emission layer 150. In an implementation, the host of the emission layer 150 may be a suitable host material when the compound for an organic electroluminescent device according to an embodiment is used as the hole transport material.

The suitable host material included in the emission layer 150 may include, e.g., tris(8-quinolinolato)aluminum (Alq₃), 4,4-N,N-dicarbazole-biphenyl (CBP), poly(n-vinylcarbazole) (PVK), 9,10-di(naphthalene-2-yl)anthracene (ADN), 4,4,4″-tris(N-carbazolyl)triphenylamine (TCTA), 1,3,5-tris(N-phenylbenzimidazole-2-yl)benzene (TPBI), 3-tert-butyl-9,10-di(naphtho-2-yl)anthracene (TBADN), distyrylarylene (DSA), 4,4-bis(9-carbazole)-2,2-dimethyl-biphenyl (dmCBP), etc.

In an implementation, the emission layer 150 may be formed as an emission layer emitting light of specific color. For example, the emission layer 150 may be formed as a red emission layer, a green emission layer, and/or a blue emission layer.

When the emission layer 150 is the blue emission layer, suitable materials may be used as a blue dopant and, e.g., perylene and a derivative thereof, an iridium (Ir) complex such as iridium(III) bis[2-(4,6-difluorophenyl)pyridinate]picolinate (FIrpic), etc.

In an implementation, when the emission layer 150 is the red emission layer, suitable materials may be used as a red dopant, e.g., rubrene and a derivative thereof, 4-dicyanomethylene-2-(p-dimethylaminostyryl)-6-methyl-4H-pyrane (DCM) and the derivative thereof, an iridium complex such as bis(1-phenylisoquinoline)(acetylacetonate)iridium(III) (Ir(piq)₂(acac)), etc., an osmium (Os) complex, a platinum (Pt) complex, etc.

In an implementation, when the emission layer 150 is the green emission layer, suitable materials may be used as a green dopant, e.g., coumarin and a derivative thereof, an iridium complex such as tris(2-phenylpyridin)iridium(III) (Ir(ppy)₃), etc.

The electron transport layer 160 may be a layer including an electron transport material provided with the function of transporting electrons and may be formed, e.g., on the emission layer 150 to a thickness from about 15 nm to about 50 nm. The electron transport layer 160 may be formed using a suitable electron transport material. The suitable electron transport material may include, e.g., a quinoline derivative such as tris(8-quinolinolato)aluminum (Alq₃), 1,2,4-triazole derivative (TAZ), bis(2-methyl-8-quinolinolato)-(p-phenylphenolate)-aluminum (BAlq), berylliumbis(benzoquinoline-10-olate) (BeBq2), a Li complex such as lithium quinolate (LiQ), etc.

The electron injection layer 170 may be a layer provided with the function of the easy injection of electrons from the second electrode 180 and may be formed to a thickness of about 0.3 nm to about 9 nm. In an implementation, the electron injection layer 170 may be formed using a suitable material, e.g., lithium fluoride (LiF), sodium chloride (NaCl), cesium fluoride (CsF), lithium oxide (Li₂O), barium oxide (BaO), etc.

The second electrode 180 may be, e.g., a cathode. For example, the second electrode 180 may be formed as a reflection type electrode using a metal having small work function, an alloy, a conductive compound, etc. The second electrode 180 may be formed using, e.g., lithium (Li), magnesium (Mg), aluminum (Al), aluminum-lithium (Al—Li), calcium (Ca), magnesium-indium (Mg—In), magnesium-silver (Mg—Ag), etc. In an implementation, the second electrode 180 may be formed as a transmission type electrode using ITO, IZO, etc. The second electrode 180 may be formed on the electron injection layer 170 using an evaporation method or a sputtering method.

An embodiment of the structure of the organic luminescent device 100 according to an embodiment has been explained. In the organic electroluminescent device 100 including the compound for an organic electroluminescent device according to an embodiment, the HOMO level of the compound for an organic electroluminescent device may be low, and stable cationic radicals may be formed. Thus, hole transport properties may be increased, and further, the driving voltage, the emission efficiency and the emission life of the organic electroluminescent device may be improved.

In an implementation, the organic electroluminescent device 100 according to an embodiment may be formed using other various suitable structures of the organic electroluminescent device. For example, the organic electroluminescent device 100 may not be provided with at least one of the hole injection layer 130, the electron transport layer 160 and the electron injection layer 170. In an implementation, any layer of the organic electroluminescent device may be formed as a single layer or a multilayer.

In an implementation, the organic electroluminescent device 100 may further include a hole blocking layer between the hole transport layer 140 and the emission layer 150 to help prevent the diffusion of triplet excitons or holes to the electron transport layer 160. In an implementation, the hole blocking layer may be formed using, e.g., an oxadiazole derivative, a triazole derivative, a phenanthroline derivative, etc.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### Examples

Herein, compounds represented by Formulae 1-1 to 1-58 (examples of the compound for an organic electroluminescent device according to an embodiment) may be referred to as Compounds 1-1 to 1-58.

### Synthetic Example 1

### Synthesis of Compound 1-2

Compound 1-2 was synthesized by the following synthetic scheme.

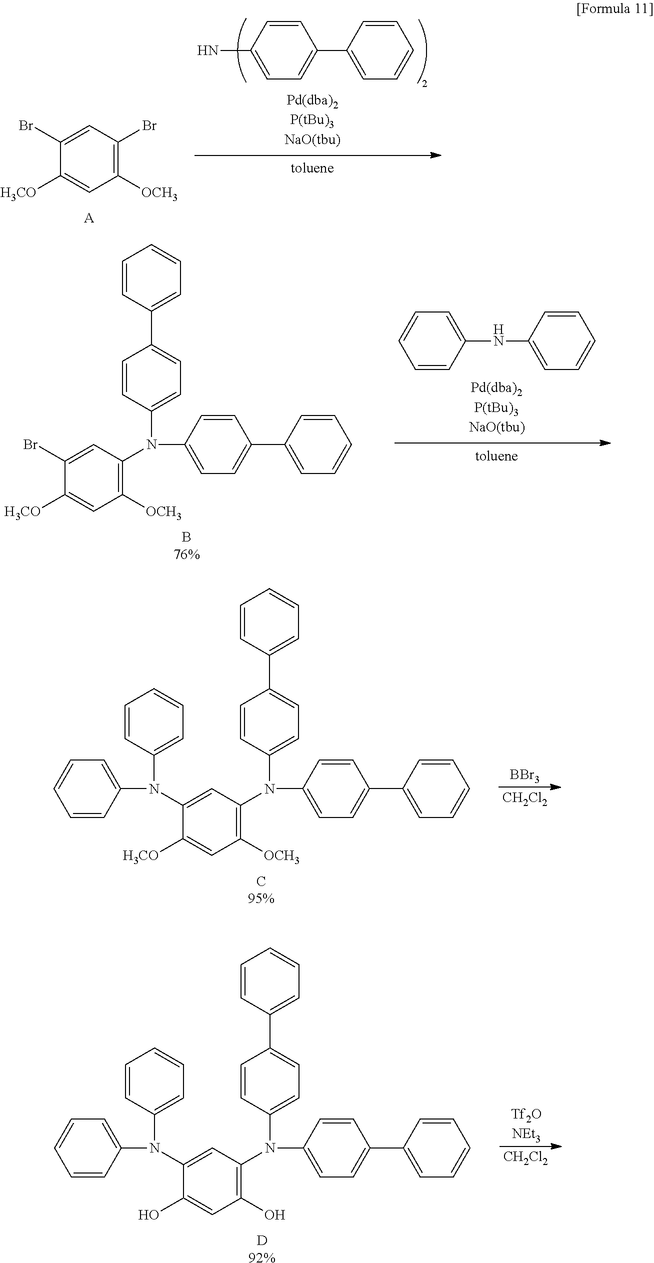

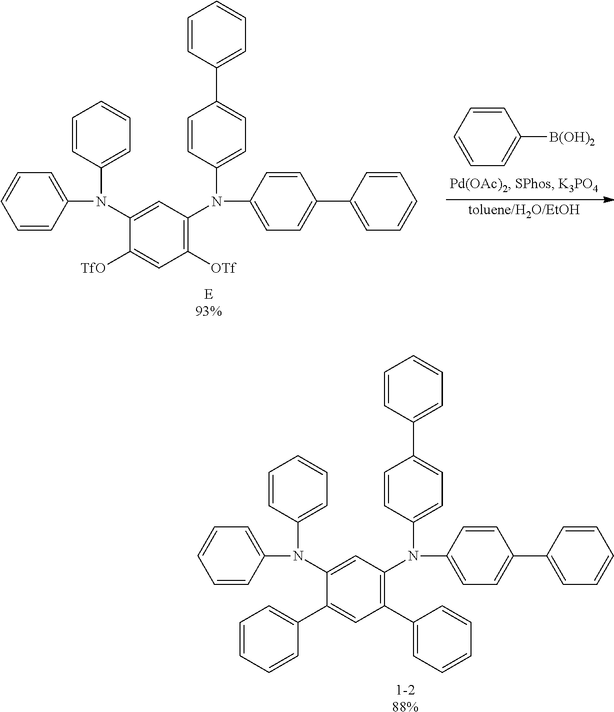

### Synthesis of Compound B

Under an argon atmosphere, 5.00 g of Compound A, 1.08 g of bis(4-biphenylyl)amine, 0.10 g of bis(dibenzylideneacetone)palladium(0), 0.14 g of tri-tert-butylphosphine and 0.49 g of sodium tert-butoxide were added to a 300 mL, three-necked flask, followed by heating and refluxing in a toluene solvent for 4 hours. After cooling in air, water was poured in the reaction mixture to separate an organic layer, and solvents were distilled. The crude product thus obtained was separated by silica gel column chromatography (toluene/hexane) to produce 1.38 g of Compound B as a white solid (yield 76%). The molecular weight of Compound B thus obtained was measured by FAB-MS and was 536 (C₃₂H₂₆BrNO₂).

### Synthesis of Compound C

Under an argon atmosphere, 1.38 g of Compound B, 0.44 g of diphenylamine, 0.07 g of bis(dibenzilideneacetone)palladium(0), 0.11 g of tri-tert-butylphosphine and 0.37 g of sodium tert-butoxide were added to a 100 mL, three-necked flask, followed by heating and refluxing in 30 mL of a toluene solvent for 4 hours. After cooling in air, water was poured in the reaction mixture to separate an organic layer, and solvents were distilled. The crude product thus obtained was separated by silica gel column chromatography (toluene) to produce 1.53 g of Compound C as a white solid (yield 95%). The molecular weight of Compound C thus obtained was measured by FAB-MS and was 624 (C₄₄H₃₆N₂O₂).

### Synthesis of Compound D

Under an argon atmosphere, 1.53 g of Compound C was dissolved in 50 mL of dichloromethane in a 200 mL, three-necked flask, and 6.2 mL of a dichloromethane solution of boron tribromide (1 mol/L) was added drop by drop in an ice bath. The reaction mixture was stirred at ambient temperature for 6 hours, and water was added to the reaction mixture to separate an organic layer, and solvents were distilled. The crude product thus obtained was separated by silica gel column chromatography (toluene and ethyl acetate) to produce 1.34 g of Compound D as a lemon yellow solid (yield 92%). The molecular weight of Compound D thus obtained was measured by FAB-MS and was 596 (C₄₂H₃₂N₂O₂).

### Synthesis of Compound E

Under an argon atmosphere, 1.34 g of Compound D was dissolved in 60 mL of dichloromethane in a 200 mL, three-necked flask, and 1.6 mL of triethylamine was added thereto in an ice bath. Then, 0.9 mL of trifluoromethylsulfonic acid anhydride was added drop by drop. The reaction mixture was stirred at ambient temperature for 2 hours, and water was added to the reaction mixture. An organic layer was separated, and solvents were distilled. The crude product thus obtained was separated by silica gel column chromatography (dichloromethane) to produce 1.79 g of Compound E as a lemon yellow solid (yield 93%). The molecular weight of Compound E thus obtained was measured by FAB-MS and was 860 (C₄₄H₃₀F₆N₂O₆S₂).

### Synthesis of Compound 1-2

Under an argon atmosphere, 1.79 g of Compound E, 0.63 g of phenylboronic acid, 0.05 g of palladium acetate, 0.17 g of 2-dicyclohexylphosphino-2,6-dimethoxybiphenyl and 1.77 g of tripotassium phosphate were added to a 200 mL, three-necked flask, followed by stirring in 25 ml of a mixture solvent of toluene, water and ethanol (10:1:1 by volume) at 100° C. for 6 hours. Water was added to the reaction mixture, an organic layer was separated, and solvents were distilled. The crude product thus obtained was separated by silica gel column chromatography (toluene and hexane) to produce 1.31 g of Compound 1-2 as a white solid (yield 88%). The molecular weight of Compound 1-2 thus obtained was measured by FAB-MS and was 716 (C₅₄H₄₀N₂). ¹H-NMR (300 MHz, DMSO-d₆) of Compound 1-2 was measured, and the following chemical shift values were obtained. 7.70-7.38 (m, 26H), 7.30-7.20 (m, 4H), 7.08 (d, J=8.5 Hz, 4H), 7.05 (d, J=8.5 Hz, 4H), 6.95 (s, 1H), 6.91 (s, 1H). Thus, the synthesis of Compound 1-2 was secured.

### Synthetic Example 2

### Synthesis of Compound 1-5

Compound 1-5 was synthesized according to the following synthetic scheme.

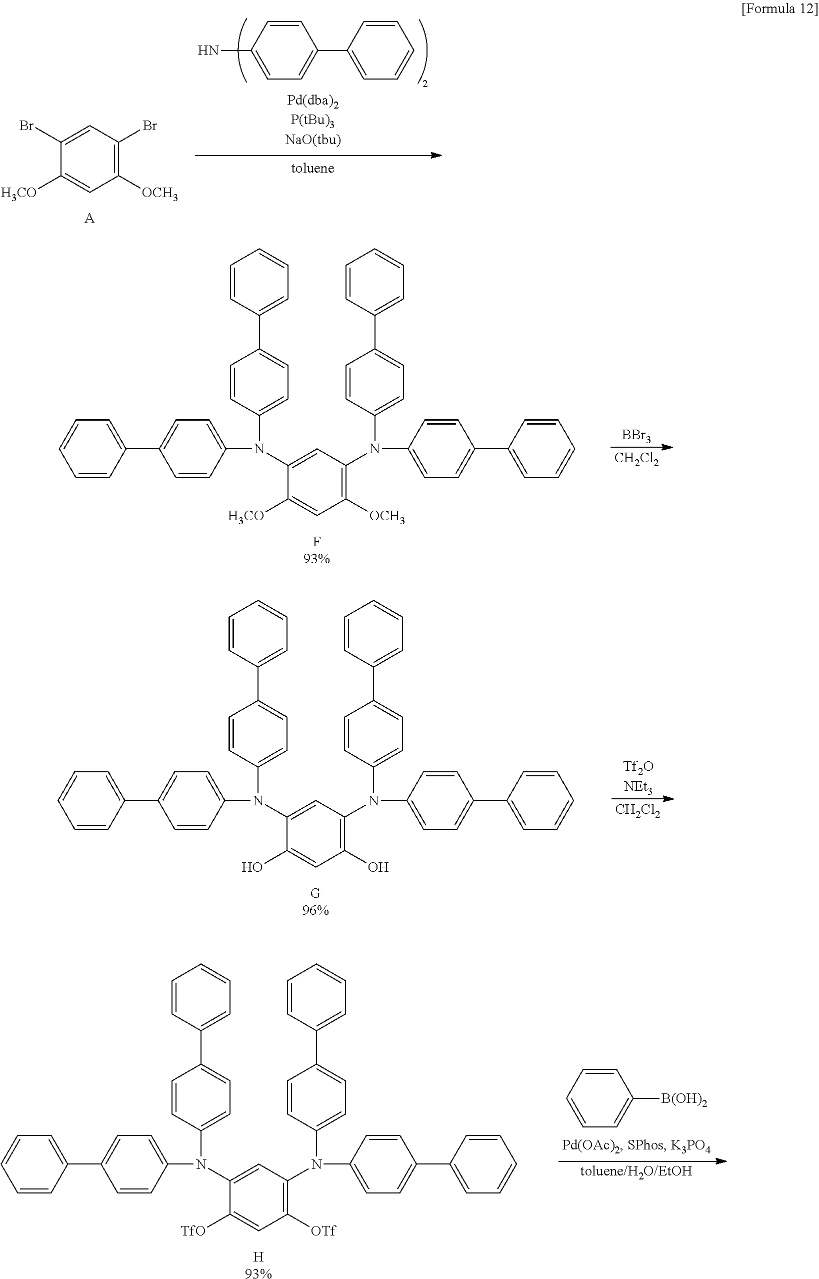

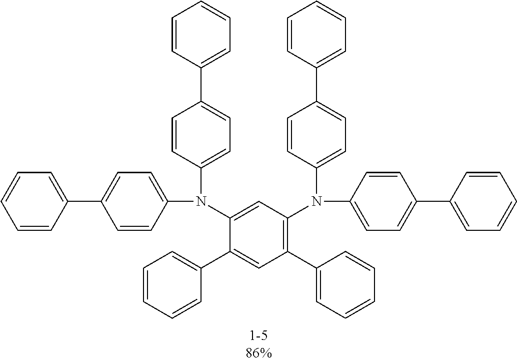 [SMILES: c1ccc(-c2ccc(N(c3ccc(-c4ccccc4)cc3)c3cc(N(c4ccc(-c5ccccc5)cc4)c4ccc(-c5ccccc5)cc4)c(-c4ccccc4)cc3-c3ccccc3)cc2)cc1 ; "1-586%"]

### Synthesis of Compound F

Under an argon atmosphere, 2.00 g of Compound A, 4.34 g of bis(4-biphenylyl)amine, 0.19 g of bis(dibenzylideneacetone)palladium(0), 0.27 g of tri-tert-butylphosphine and 1.62 g of sodium tert-butoxide were added to a 200 mL, three-necked flask, followed by heating and refluxing in 70 mL of a toluene solvent for 4 hours. After cooling in air, water was added to the reaction mixture, an organic layer was separated, and solvents were distilled. The crude product thus obtained was separated by silica gel column chromatography (toluene) to produce 4.88 g of Compound F as a white solid (yield 93%). The molecular weight of Compound F thus obtained was measured by FAB-MS and was 776 (C₅₅H₄₄N₂O₂).

### Synthesis of Compound G

Under an argon atmosphere, 3.00 g of Compound F was dissolved in 100 mL of dichloromethane in a 200 mL, three-necked flask, and 9.7 mL of a dichloromethane solution of boron tribromide (1 mol/L) was added thereto drop by drop in an ice bath. The reaction mixture was stirred at ambient temperature for 6 hours, water was added to separate an organic layer, and solvents were distilled. The crude product thus obtained was separated by silica gel column chromatography (toluene and ethyl acetate) to produce 2.77 g of Compound G as a lemon yellow solid (yield 96%). The molecular weight of Compound G thus obtained was measured by FAB-MS and was 748 (C₅₄H₄₀N₂O₂).

### Synthesis of Compound H

Under an argon atmosphere, 2.77 g of Compound G was dissolved in 60 mL of dichloromethane in a 200 mL, three-necked flask, and 2.6 mL of triethylamine was added thereto in an ice bath. Then, 1.6 ml of trifluoromethylsulfonic acid anhydride was added thereto drop by drop. After stirring at ambient temperature for 2 hours, water was added to separate an organic layer, and solvents were distilled. The crude product thus obtained was separated by silica gel column chromatography (dichloromethane) to produce 3.48 g of Compound H as a lemon yellow solid (yield 93%). The molecular weight of Compound H thus obtained was measured by FAB-MS and was 1013 (C₅₆H₃₈F₆N₂O₆S₂).

### Synthesis of Compound 1-5

Under an argon atmosphere, 3.48 g of Compound H, 1.05 g of phenylboronic acid, 0.08 g of palladium acetate, 0.28 g of 2-dicyclohexylphosphino-2,6-dimethoxybiphenyl and 2.92 g of tripotassium phosphate were added to a 200 mL, three-necked flask, followed by stirring in 40 mL of a mixture solvent of toluene, water and ethanol (10:1:1 by volume) at 100° C. for 6 hours. Water was added to the reaction mixture, an organic layer was separated, and solvents were distilled. The crude product thus obtained was separated by silica gel column chromatography (toluene and hexane) to produce 2.56 g of Compound 1-5 as a white solid (yield 86%). The molecular weight of Compound 1-5 thus obtained was measured by FAB-MS and was 869 (C₆₆H₄₈N₂). ¹H-NMR (300 MHz, DMSO-d₆) of Compound 1-5 was measured, and the following chemical shift values were obtained. 7.71-7.35 (m, 34H), 7.23 (d, J=8.3 Hz, 4H), 7.02 (d, J=8.5 Hz, 8H), 6.97 (s, 1H), 6.94 (s, 1H). Thus, the synthesis of Compound 1-5 was secured.

### Synthetic Example 3

### Synthesis of Compound 1-7

In the synthetic scheme of Compound 1-5, N-phenyl-1-naphthylamine was used instead of bis(4-biphenylyl)amine to synthesize Compound 1-7. In addition, the molecular weight of Compound 1-7 was measured by FAB-MS, and a value of 664 (C₅₀H₃₆N₂) was obtained. In addition, ¹H NMR (300 MHz, DMSO-d₆) of Compound 1-7 was measured, and expected chemical shift values from the structure of Compound 1-7 were obtained. Thus, the synthesis of Compound 1-7 was secured.

### Synthetic Example 4

### Synthesis of Compound 1-10

In the synthetic scheme of Compound 1-5, 3-(biphenylamino)dibenzofuran was used instead of bis(4-biphenylyl)amine to synthesize Compound 1-10. In addition, the molecular weight of Compound 1-10 was measured by FAB-MS, and a value of 896 (C₆₆H₄₄N₂O₂) was obtained. In addition, ¹H NMR (300 MHz, DMSO-d₆) of Compound 1-10 was measured, and expected chemical shift values from the structure of Compound 1-10 were obtained. Thus, the synthesis of Compound 1-10 was secured.

### Synthetic Example 5

### Synthesis of Compound 1-16

In the synthetic scheme of Compound 1-5, 3-(biphenylamino)dibenzothiophene was used instead of bis(4-biphenylyl)amine to synthesize Compound 1-16. In addition, the molecular weight of Compound 1-16 was measured by FAB-MS, and a value of 928 (C₆₆H₄₄N₂S₂) was obtained. In addition, ¹H NMR (300 MHz, DMSO-d₆) of Compound 1-16 was measured, and expected chemical shift values from the structure of Compound 1-16 were obtained. Thus, the synthesis of Compound 1-16 was secured.

### Synthetic Example 6

### Synthesis of Compound 1-37

In the synthetic scheme of Compound 1-5, 3-bromodibenzofuran was used instead of phenylboronic acid to synthesize Compound 1-37. In addition, the molecular weight of Compound 1-37 was measured by FAB-MS, and a value of 1048 (C₇₈H₅₂N₂O₂) was obtained. In addition, ¹H NMR (300 MHz, DMSO-d₆) of Compound 1-37 was measured, and expected chemical shift values from the structure of Compound 1-37 were obtained. Thus, the synthesis of Compound 1-37 was secured.

(Measuring of HOMO Level)

Compounds 1-2, 1-5, 1-7, 1-10, 1-16, and 1-37 are compounds illustrated above. The HOMO levels of Compounds 1-2, 1-5, 1-7, 1-10, 1-16, and 1-37 were measured using AC-3 (a photoelectron spectrometer in the atmosphere, manufactured by RIKEN KEIKI Co., Ltd.). In addition, the HOMO level of Compound C1 was measured for comparison. Compound C1 is illustrated below. Compound C1 is presented as a comparative example. The measured results are illustrated in Table 1.

TABLE 1

| | HOMO level (eV) |
|---|---|
| Compound 1-2 | −5.70 |
| Compound 1-5 | −5.72 |
| Compound 1-7 | −5.70 |
| Compound 1-10 | −5.75 |
| Compound 1-16 | −5.76 |
| Compound 1-37 | −5.77 |
| Compound C1 | −5.30 |

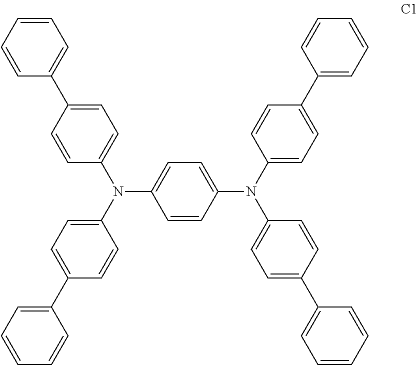 [SMILES: c1ccc(-c2ccc(N(c3ccc(-c4ccccc4)cc3)c3ccc(N(c4ccc(-c5ccccc5)cc4)c4ccc(-c5ccccc5)cc4)cc3)cc2)cc1]

As may be seen in Table 1, the HOMO levels of Compounds 1-2, 1-5, 1-7, 1-10, 1-16, and 1-37 are lower than the HOMO level of Compound C1. Thus, in organic electroluminescent devices including Compounds 1-2, 1-5, 1-7, 1-10, 1-16, or 1-37 as a hole transport material, it may be thought that holes may be smoothly injected from a hole injection layer to a hole transport layer.

(Measuring of Glass Transition Temperature)

Then, the glass transition temperatures of Compounds 1-2, 1-5, 1-7, 1-10, 1-16, and 1-37 were measured using a differential scanning calorimetry, DSC7020 manufactured by Hitachi High-Technologies Corporation. In addition, the glass transition temperature of Compound C2, illustrated below, was also measured for comparison. Compound C2 was used as a comparative example. Measured results are illustrated in Table 2.

TABLE 2

| | Glass transition |
|---|---|
| | temperature (° C.) |
| Compound 1-2 | 115 |
| Compound 1-5 | 128 |
| Compound 1-7 | 103 |
| Compound 1-10 | 125 |
| Compound 1-16 | 132 |
| Compound 1-37 | 138 |
| Compound C2 | 91 |

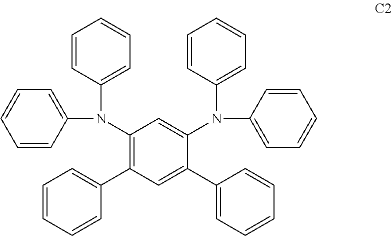 [SMILES: c1ccc(-c2cc(-c3ccccc3)c(N(c3ccccc3)c3ccccc3)cc2N(c2ccccc2)c2ccccc2)cc1]

As may be seen in Table 2, the glass transition temperatures of Compounds 1-2, 1-5, 1-7, 1-10, 1-16, and 1-37 are higher than the glass transition temperature of Compound C2. Thus, it would be thought that the thermal stability of Compounds 1-2, 1-5, 1-7, 1-10, 1-16, and 1-37 was increased in organic electroluminescent devices using Compounds 1-2, 1-5, 1-7, 1-10, 1-16 and 1-37 as hole transport materials, and layer quality in the device was improved.

(Manufacturing of Organic Electroluminescent Device)

An organic electroluminescent device was manufactured by the following method. First, an ITO-glass substrate patterned and washed in advance was surface treated with ozone (O₃). The layer thickness of an ITO layer (a first electrode) was about 150 nm. After ozone treatment, the substrate was washed. The washed substrate was installed in a glass bell jar-type evaporator for forming an organic layer, and a hole injection layer, a hole transport layer HTL, an emission layer and an electron transport layer were deposited one by one under a vacuum degree of about 10⁻⁴ to about 10⁻⁵ Pa. The material of the hole injection layer was 2-TNATA, and the thickness thereof was about 60 nm. The materials of HTL were compounds represented in Table 3, and the thickness thereof was about 30 nm. In addition, the thickness of the emission layer was about 25 nm. The host of an emission material was ADN, and the dopant thereof was TBP. The amount doped of the dopant was about 3 wt % with respect to the amount of the host. The material of the electron transport layer was Alq3, and the thickness thereof was about 25 nm. Continuously, the substrate was transported to the glass bell jar-type evaporator for forming a metal layer, and materials for an electron injection layer and a cathode were deposited one by one under a vacuum degree of about 10⁻⁴ to about 10⁻⁵ Pa. The material of the electron injection layer was LiF, and the thickness thereof was about 1.0 nm. The material of the second electrode was Al, and the thickness thereof was about 100 nm.

TABLE 3

| | | | | Emis- | Emis- |
|---|---|---|---|---|---|
| | | | | sion | sion |
| | | Current | Driving | effi- | life |
| | | density | voltage | ciency | LT50 |
| | HTL | [mA/cm²] | [V] | [cd/A] | (hr) |
| Example 1 | Compound 1-2 | 10 | 6. | 6.7 | 1,800 |
| Example 2 | Compound 1-5 | 10 | 6.3 | 7.1 | 2,100 |
| Example 3 | Compound 1-7 | 10 | 6.4 | 7.0 | 1,850 |
| Example 4 | Compound 1-10 | 10 | 6.5 | 7.2 | 1,900 |
| Example 5 | Compound 1-16 | 10 | 6.4 | 7.2 | 1,700 |
| Example 6 | Compound 1-37 | 10 | 6.3 | 7.0 | 1,900 |
| Comparative | Compound C1 | 10 | 8.1 | 5.3 | 1,100 |
| Example 1 | | | | | |
| Comparative | Compound C2 | 10 | 6.7 | 5.9 | 1,500 |
| Example 2 | | | | | |

(Evaluation of Properties)

Then, the driving voltage, the emission efficiency, and the emission life of the organic electroluminescent devices thus manufactured were measured. For the evaluation of the electroluminescent properties of the organic electroluminescent devices 100 thus manufactured, a brightness light distribution characteristics measurement system of C9920-11 manufactured by HAMAMATSU Photonics Co. was used. In addition, in the above Table 3, the current density was measured at 10 mA/cm², and the half life was measured at 1,000 cd/m². The results are shown in Table 3.

Referring to Table 3, a driving voltage was decreased, and efficiency and life were increased for the organic electroluminescent devices according to Examples 1 to 6, when compared to the organic electroluminescent devices according to Comparative Examples 1 and 2. For example, the driving voltage and the emission efficiency of the organic electroluminescent devices according to Examples 1 to 6 were largely improved when compared to those of the organic electroluminescent device according to Comparative Example 1. It would be considered that the HOMO levels of the materials for the organic electroluminescent devices of Examples 1 to 6 are low and appropriate values for the organic electroluminescent devices. In addition, the efficiency and the emission life of the organic electroluminescent devices of Examples 1 to 6 were largely improved when compared to those of the organic electroluminescent device of Comparative Example 2. In would be considered that the glass transition temperature of the materials for the organic electroluminescent devices of Examples 1 to 6 is increased, the stability of the molecules themselves was increased, and the quality of the layer was improved. In addition, it would be considered that the stability of cationic radicals was improved through the substituent on nitrogen from a phenyl group to a biphenyl group, a dibenzoheterole group, etc. As described above, the driving voltage, the emission efficiency and the emission life of the organic electroluminescent device were largely improved particularly in a blue emission region in an embodiment. In addition, the compounds of the embodiments have a wide energy gap, which may correspond to a blue emission region, the application thereof in green to red regions may be also possible.

By way of summation and review, a hole transport material used in the hole transport layer may include, e.g., a p-phenylenediamine derivative, and a charge transport material used in an electrophotographic photoreceptor may include, e.g., a m-phenylenediamine derivative.

An organic electroluminescent device using the p-phenylenediamine derivative as the hole transport material may not provide satisfactory values of a driving voltage, emission efficiency, and emission life. In addition, an organic electroluminescent device manufactured by using the m-phenylenediamine derivative as the hole transport material may not provide satisfactory values of a driving voltage, emission efficiency, and emission life. As described above, satisfactory driving voltage, emission efficiency, and emission life may not be obtained by using the derivatives in the organic electroluminescent device.

The embodiments may provide a compound for an organic electroluminescent device capable of improving the driving voltage, the emission efficiency, and the emission life of the organic electroluminescent device.

The compound for an organic electroluminescent device may have a low level of the highest occupied molecular orbital (HOMO) and a high glass transition temperature, the driving voltage, the emission efficiency and the emission life of the organic electroluminescent device may be improved by using the compound in the organic electroluminescent device.

As described above, the compound for an organic electroluminescent device according to an embodiment may be represented by Formula 1, and the driving voltage, the emission efficiency, and the emission life of the organic electroluminescent device including the same may be largely improved. Thus, the compound for an organic electroluminescent device according to an embodiment may be useful for the practical application of diverse purposes.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic electroluminescent device, the compound being represented by the following Formula 1: 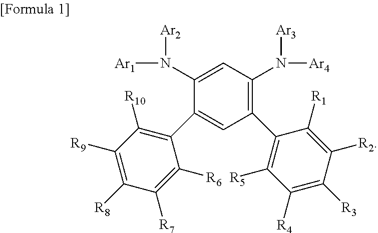
wherein, in Formula 1,
Ar₁ to Ar₄ are each independently a substituted or unsubstituted aryl group or a substituted or unsubstituted heteroaryl group, at least one of Ar₁ to Ar₄ being a substituted or unsubstituted aryl group having at least 10 ring carbon atoms or a substituted or unsubstituted heteroaryl group having at least 10 ring carbon atoms,
R₁ to R₁₀ are each independently a hydrogen atom, a substituted or unsubstituted aryl group, a substituted or unsubstituted heteroaryl group, a substituted or unsubstituted alkyl group, a substituted or unsubstituted alkoxy group, a halogen atom, an electron withdrawing group, or a deuterium atom, and
R₁ to R₁₀ are separate or adjacent ones thereof are combined to each other to form a substituted or unsubstituted ring.

2. The compound for an organic electroluminescent device as claimed in claim 1, wherein the aryl group having at least 10 ring carbon atoms is a biphenyl group, a naphthyl group, a phenanthryl group, or a triphenylenyl group.

3. The compound for an organic electroluminescent device as claimed in claim 2, wherein the aryl group having at least 10 ring carbon atoms is a biphenyl group.

4. The compound for an organic electroluminescent device as claimed in claim 1, wherein the heteroaryl group having at least 10 ring carbon atoms is a dibenzoheterole group.

5. The compound for an organic electroluminescent device as claimed in claim 4, wherein the dibenzoheterole group is represented by the following Formula 2: 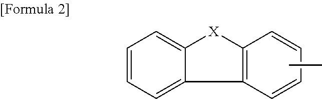
wherein, in Formula 2, X is one of O, S, or Si(R₁₁)₂, in which each R₁₁ is independently a hydrogen, a substituted or unsubstituted alkyl group, or a substituted or unsubstituted aryl group.

6. The compound for an organic electroluminescent device as claimed in claim 1, wherein Ar₁ to Ar₄ are each independently a substituted or unsubstituted aryl group having at least 10 ring carbon atoms or a substituted or unsubstituted heteroaryl group having at least 10 ring carbon atoms.

7. The compound for an organic electroluminescent device as claimed in claim 1, wherein the compound includes one of the following Compounds 1-1 to 1-24:

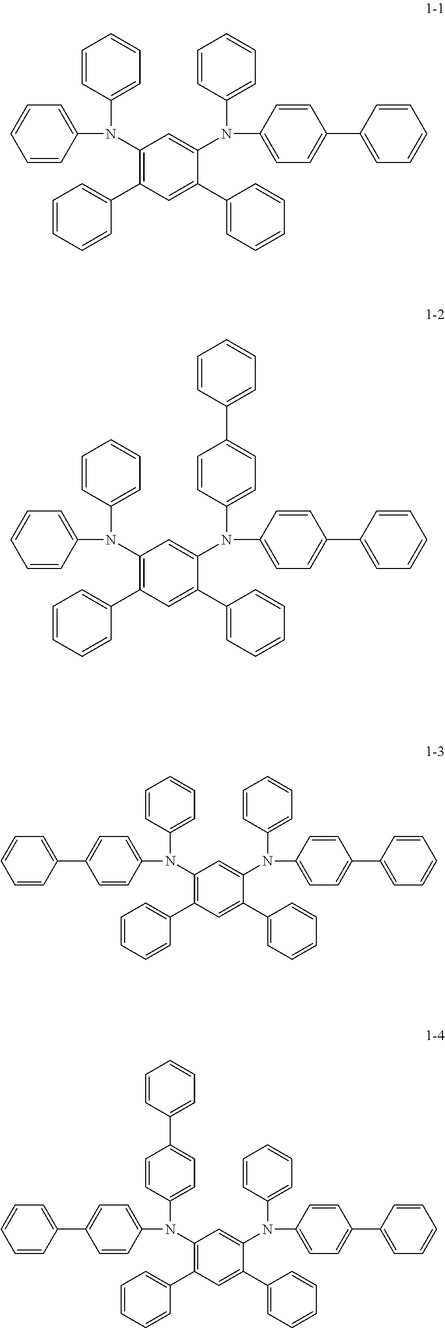 [SMILES: c1ccc(-c2ccc(N(c3ccccc3)c3cc(N(c4ccccc4)c4ccccc4)c(-c4ccccc4)cc3-c3ccccc3)cc2)cc1 ; c1ccc(-c2ccc(N(c3ccc(-c4ccccc4)cc3)c3cc(N(c4ccccc4)c4ccccc4)c(-c4ccccc4)cc3-c3ccccc3)cc2)cc1 ; c1ccc(-c2ccc(N(c3ccccc3)c3cc(N(c4ccccc4)c4ccc(-c5ccccc5)cc4)c(-c4ccccc4)cc3-c3ccccc3)cc2)cc1 ; c1ccc(-c2ccc(N(c3ccccc3)c3cc(N(c4ccc(-c5ccccc5)cc4)c4ccc(-c5ccccc5)cc4)c(-c4ccccc4)cc3-c3ccccc3)cc2)cc1]

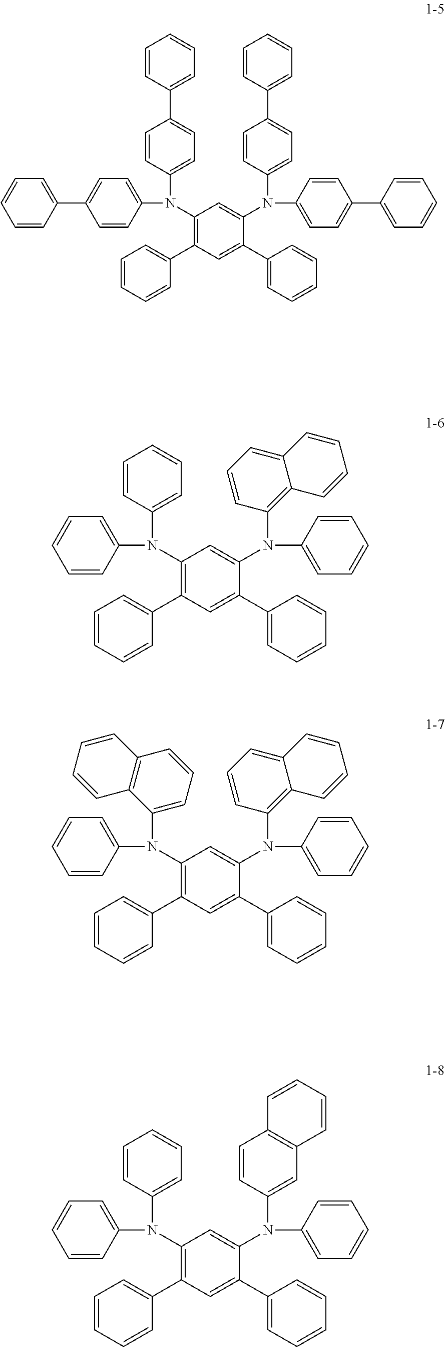 [SMILES: c1ccc(-c2ccc(N(c3ccc(-c4ccccc4)cc3)c3cc(N(c4ccc(-c5ccccc5)cc4)c4ccc(-c5ccccc5)cc4)c(-c4ccccc4)cc3-c3ccccc3)cc2)cc1 ; c1ccc(-c2cc(-c3ccccc3)c(N(c3ccccc3)c3cccc4ccccc34)cc2N(c2ccccc2)c2ccccc2)cc1 ; c1ccc(-c2cc(-c3ccccc3)c(N(c3ccccc3)c3cccc4ccccc34)cc2N(c2ccccc2)c2cccc3ccccc23)cc1 ; c1ccc(-c2cc(-c3ccccc3)c(N(c3ccccc3)c3ccc4ccccc4c3)cc2N(c2ccccc2)c2ccccc2)cc1]

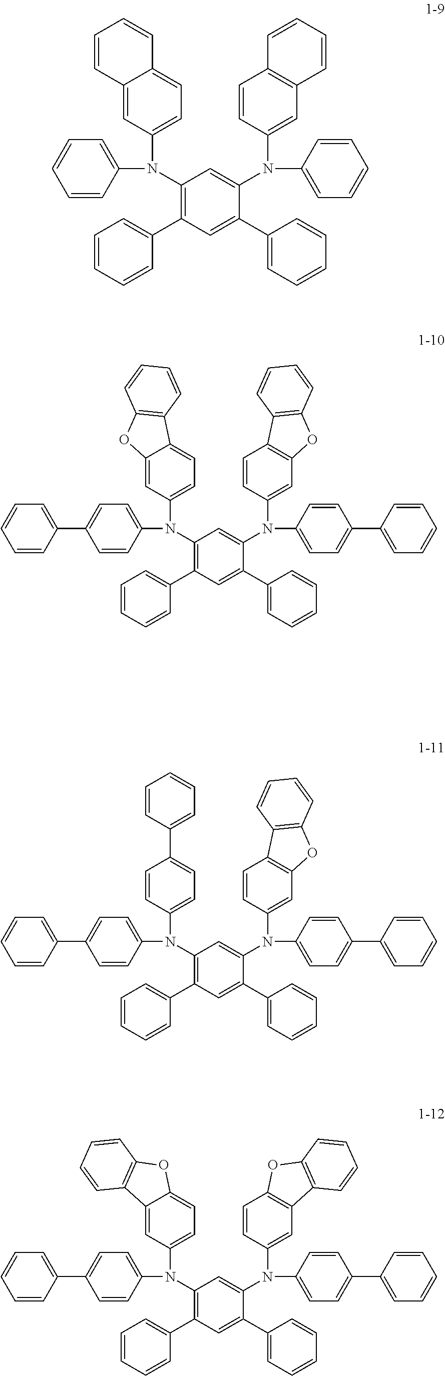 [SMILES: c1ccc(-c2cc(-c3ccccc3)c(N(c3ccccc3)c3ccc4ccccc4c3)cc2N(c2ccccc2)c2ccc3ccccc3c2)cc1 ; c1ccc(-c2ccc(N(c3ccc4c(c3)oc3ccccc34)c3cc(N(c4ccc(-c5ccccc5)cc4)c4ccc5c(c4)oc4ccccc45)c(-c4ccccc4)cc3-c3ccccc3)cc2)cc1 ; c1ccc(-c2ccc(N(c3ccc(-c4ccccc4)cc3)c3cc(N(c4ccc(-c5ccccc5)cc4)c4ccc5c(c4)oc4ccccc45)c(-c4ccccc4)cc3-c3ccccc3)cc2)cc1 ; c1ccc(-c2ccc(N(c3ccc4oc5ccccc5c4c3)c3cc(N(c4ccc(-c5ccccc5)cc4)c4ccc5oc6ccccc6c5c4)c(-c4ccccc4)cc3-c3ccccc3)cc2)cc1]

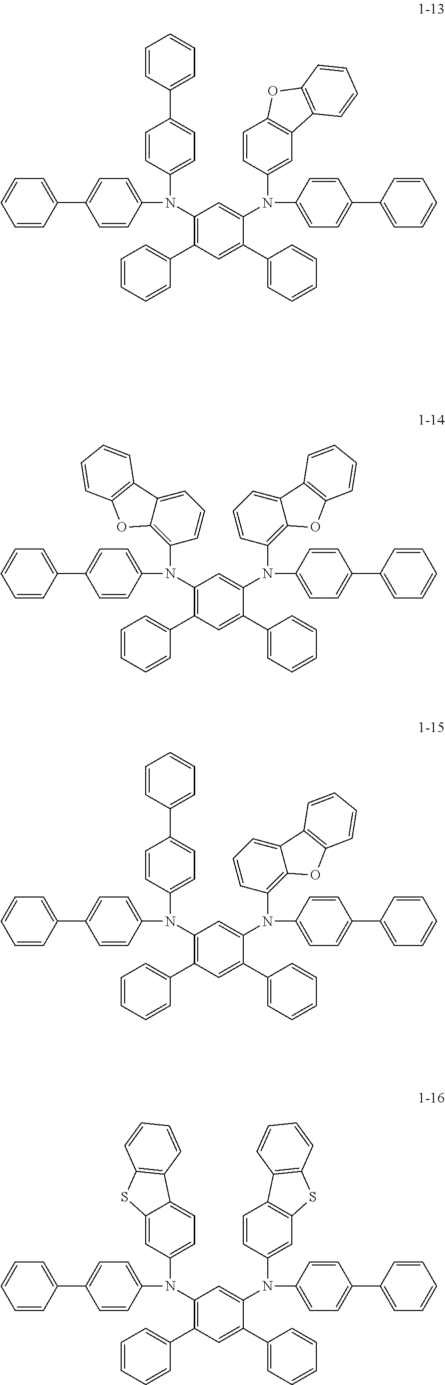 [SMILES: c1ccc(-c2ccc(N(c3ccc(-c4ccccc4)cc3)c3cc(N(c4ccc(-c5ccccc5)cc4)c4ccc5oc6ccccc6c5c4)c(-c4ccccc4)cc3-c3ccccc3)cc2)cc1 ; c1ccc(-c2ccc(N(c3cc(N(c4ccc(-c5ccccc5)cc4)c4cccc5c4oc4ccccc45)c(-c4ccccc4)cc3-c3ccccc3)c3cccc4c3oc3ccccc34)cc2)cc1 ; c1ccc(-c2ccc(N(c3ccc(-c4ccccc4)cc3)c3cc(N(c4ccc(-c5ccccc5)cc4)c4cccc5c4oc4ccccc45)c(-c4ccccc4)cc3-c3ccccc3)cc2)cc1 ; c1ccc(-c2ccc(N(c3ccc4c(c3)sc3ccccc34)c3cc(N(c4ccc(-c5ccccc5)cc4)c4ccc5c(c4)sc4ccccc45)c(-c4ccccc4)cc3-c3ccccc3)cc2)cc1]

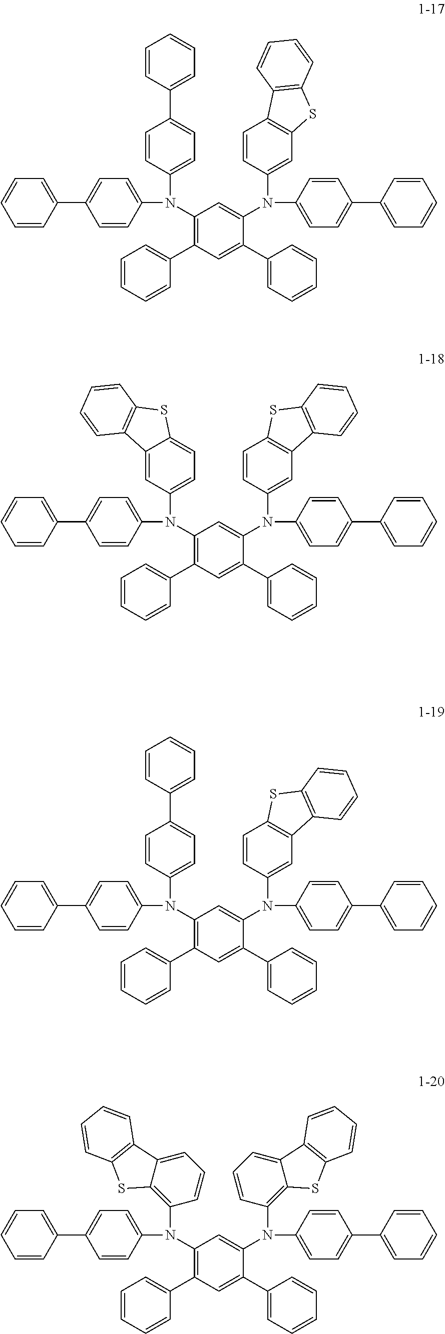 [SMILES: c1ccc(-c2ccc(N(c3ccc(-c4ccccc4)cc3)c3cc(N(c4ccc(-c5ccccc5)cc4)c4ccc5c(c4)sc4ccccc45)c(-c4ccccc4)cc3-c3ccccc3)cc2)cc1 ; c1ccc(-c2ccc(N(c3ccc4sc5ccccc5c4c3)c3cc(N(c4ccc(-c5ccccc5)cc4)c4ccc5sc6ccccc6c5c4)c(-c4ccccc4)cc3-c3ccccc3)cc2)cc1 ; c1ccc(-c2ccc(N(c3ccc(-c4ccccc4)cc3)c3cc(N(c4ccc(-c5ccccc5)cc4)c4ccc5sc6ccccc6c5c4)c(-c4ccccc4)cc3-c3ccccc3)cc2)cc1 ; c1ccc(-c2ccc(N(c3cc(N(c4ccc(-c5ccccc5)cc4)c4cccc5c4sc4ccccc45)c(-c4ccccc4)cc3-c3ccccc3)c3cccc4c3sc3ccccc34)cc2)cc1]

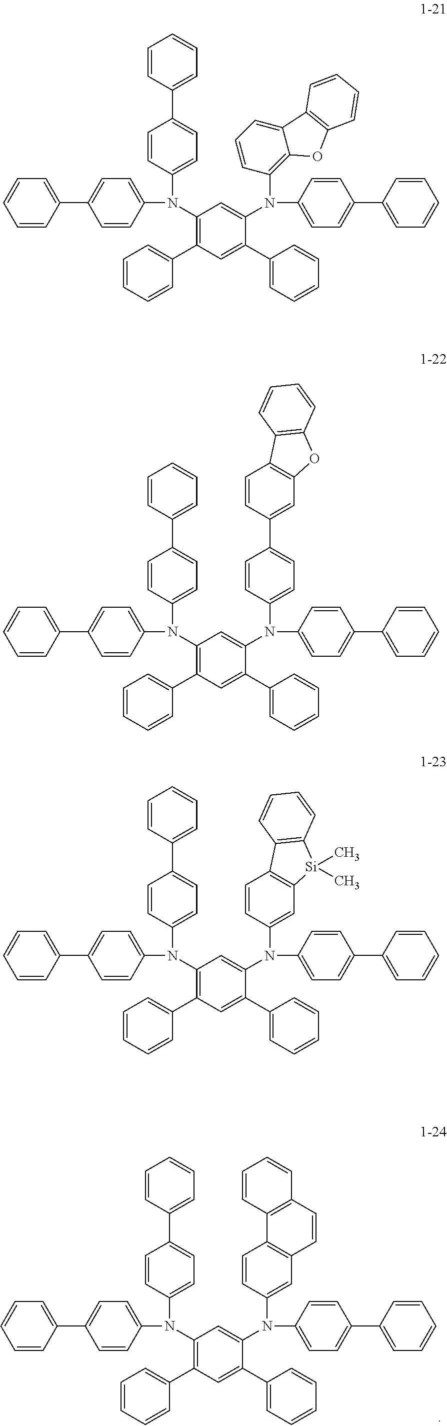 [SMILES: c1ccc(-c2ccc(N(c3ccc(-c4ccccc4)cc3)c3cc(N(c4ccc(-c5ccccc5)cc4)c4cccc5c4oc4ccccc45)c(-c4ccccc4)cc3-c3ccccc3)cc2)cc1 ; c1ccc(-c2ccc(N(c3ccc(-c4ccccc4)cc3)c3cc(N(c4ccc(-c5ccccc5)cc4)c4ccc(-c5ccc6c(c5)oc5ccccc56)cc4)c(-c4ccccc4)cc3-c3ccccc3)cc2)cc1 ; C[Si]1(C)c2ccccc2-c2ccc(N(c3ccc(-c4ccccc4)cc3)c3cc(N(c4ccc(-c5ccccc5)cc4)c4ccc(-c5ccccc5)cc4)c(-c4ccccc4)cc3-c3ccccc3)cc21 ; c1ccc(-c2ccc(N(c3ccc(-c4ccccc4)cc3)c3cc(N(c4ccc(-c5ccccc5)cc4)c4ccc5c(ccc6ccccc65)c4)c(-c4ccccc4)cc3-c3ccccc3)cc2)cc1]

8. The compound for an organic electroluminescent device as claimed in claim 1, wherein the compound includes one of the following Compounds 1-25 to 1-46:

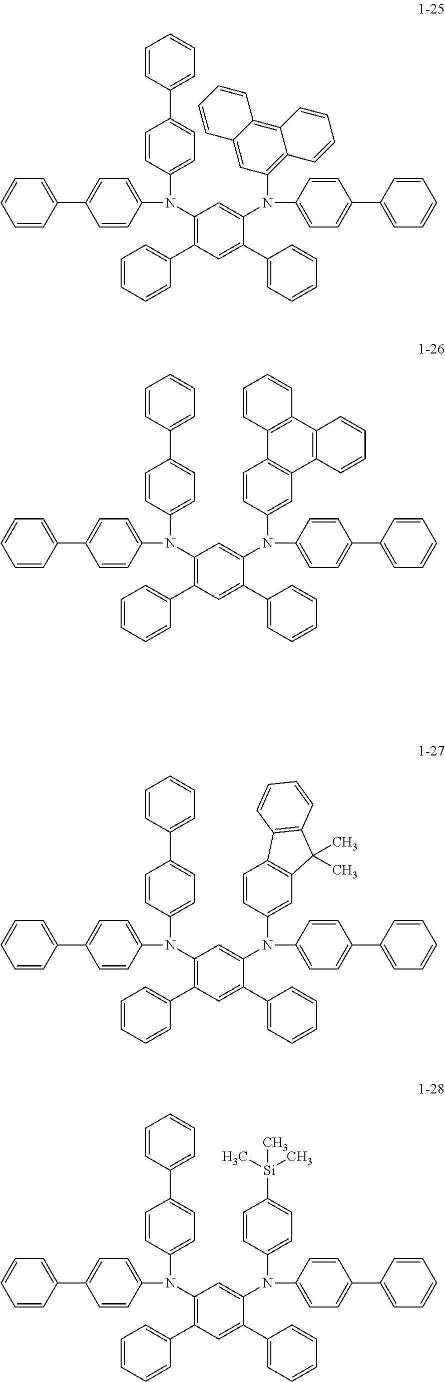 [SMILES: c1ccc(-c2ccc(N(c3ccc(-c4ccccc4)cc3)c3cc(N(c4ccc(-c5ccccc5)cc4)c4cc5ccccc5c5ccccc45)c(-c4ccccc4)cc3-c3ccccc3)cc2)cc1 ; c1ccc(-c2ccc(N(c3ccc(-c4ccccc4)cc3)c3cc(N(c4ccc(-c5ccccc5)cc4)c4ccc5c6ccccc6c6ccccc6c5c4)c(-c4ccccc4)cc3-c3ccccc3)cc2)cc1 ; CC1(C)c2ccccc2-c2ccc(N(c3ccc(-c4ccccc4)cc3)c3cc(N(c4ccc(-c5ccccc5)cc4)c4ccc(-c5ccccc5)cc4)c(-c4ccccc4)cc3-c3ccccc3)cc21 ; C[Si](C)(C)c1ccc(N(c2ccc(-c3ccccc3)cc2)c2cc(N(c3ccc(-c4ccccc4)cc3)c3ccc(-c4ccccc4)cc3)c(-c3ccccc3)cc2-c2ccccc2)cc1]

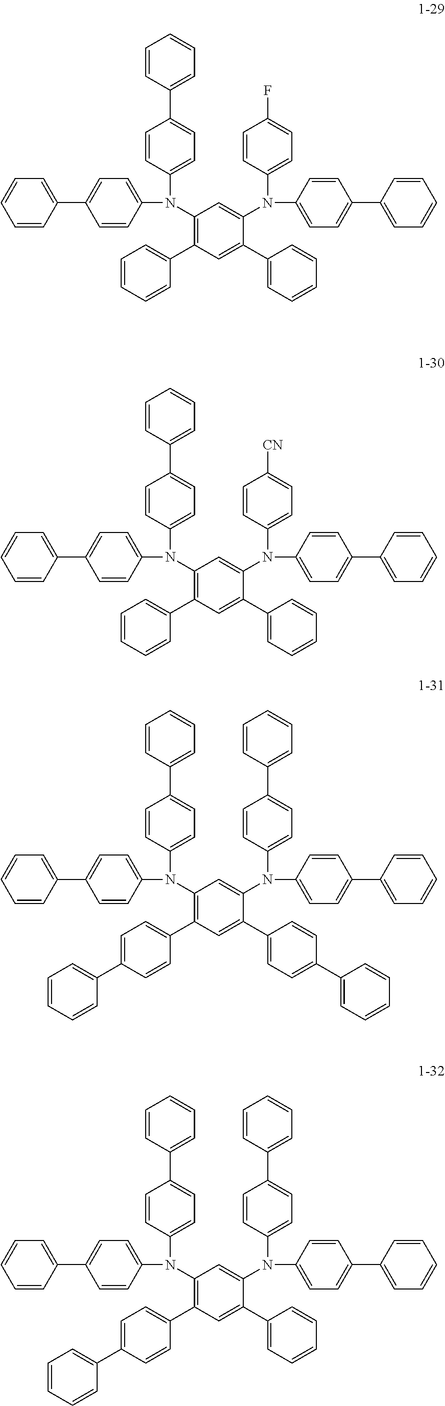 [SMILES: Fc1ccc(N(c2ccc(-c3ccccc3)cc2)c2cc(N(c3ccc(-c4ccccc4)cc3)c3ccc(-c4ccccc4)cc3)c(-c3ccccc3)cc2-c2ccccc2)cc1 ; N#Cc1ccc(N(c2ccc(-c3ccccc3)cc2)c2cc(N(c3ccc(-c4ccccc4)cc3)c3ccc(-c4ccccc4)cc3)c(-c3ccccc3)cc2-c2ccccc2)cc1 ; c1ccc(-c2ccc(-c3cc(-c4ccc(-c5ccccc5)cc4)c(N(c4ccc(-c5ccccc5)cc4)c4ccc(-c5ccccc5)cc4)cc3N(c3ccc(-c4ccccc4)cc3)c3ccc(-c4ccccc4)cc3)cc2)cc1 ; c1ccc(-c2ccc(-c3cc(-c4ccccc4)c(N(c4ccc(-c5ccccc5)cc4)c4ccc(-c5ccccc5)cc4)cc3N(c3ccc(-c4ccccc4)cc3)c3ccc(-c4ccccc4)cc3)cc2)cc1]

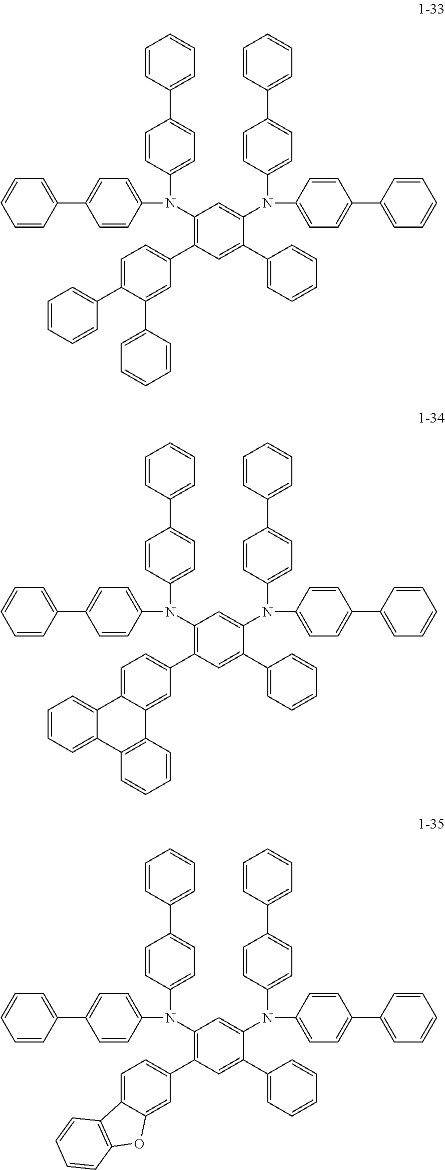 [SMILES: c1ccc(-c2ccc(N(c3ccc(-c4ccccc4)cc3)c3cc(N(c4ccc(-c5ccccc5)cc4)c4ccc(-c5ccccc5)cc4)c(-c4ccc(-c5ccccc5)c(-c5ccccc5)c4)cc3-c3ccccc3)cc2)cc1 ; c1ccc(-c2ccc(N(c3ccc(-c4ccccc4)cc3)c3cc(N(c4ccc(-c5ccccc5)cc4)c4ccc(-c5ccccc5)cc4)c(-c4ccc5c6ccccc6c6ccccc6c5c4)cc3-c3ccccc3)cc2)cc1 ; c1ccc(-c2ccc(N(c3ccc(-c4ccccc4)cc3)c3cc(N(c4ccc(-c5ccccc5)cc4)c4ccc(-c5ccccc5)cc4)c(-c4ccc5c(c4)oc4ccccc45)cc3-c3ccccc3)cc2)cc1]

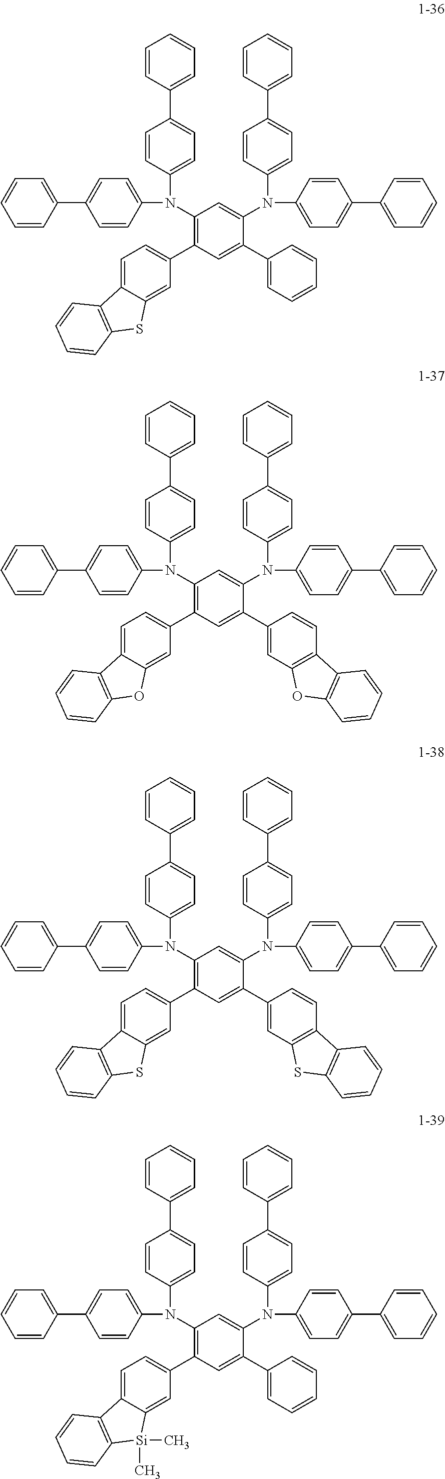

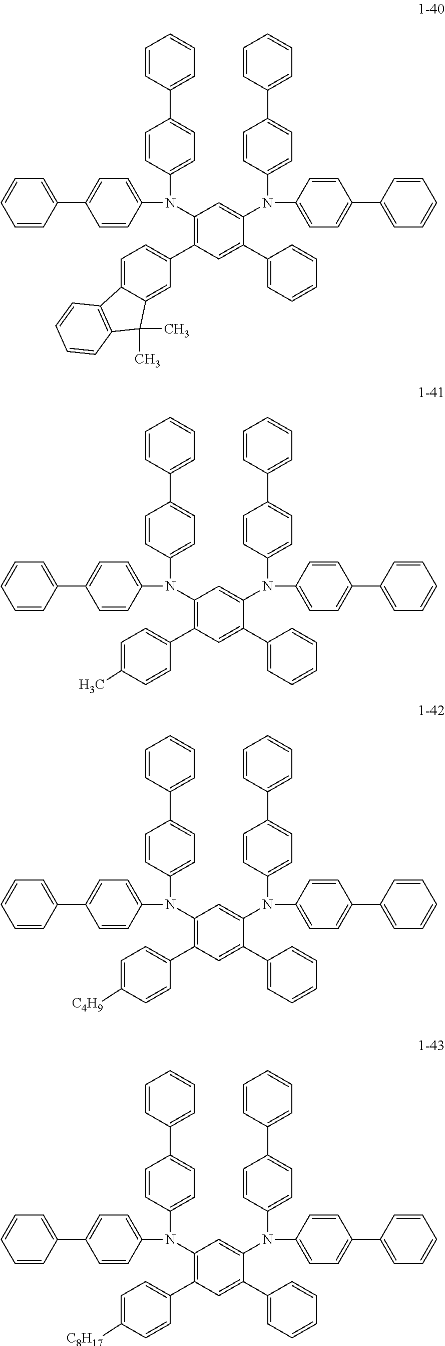

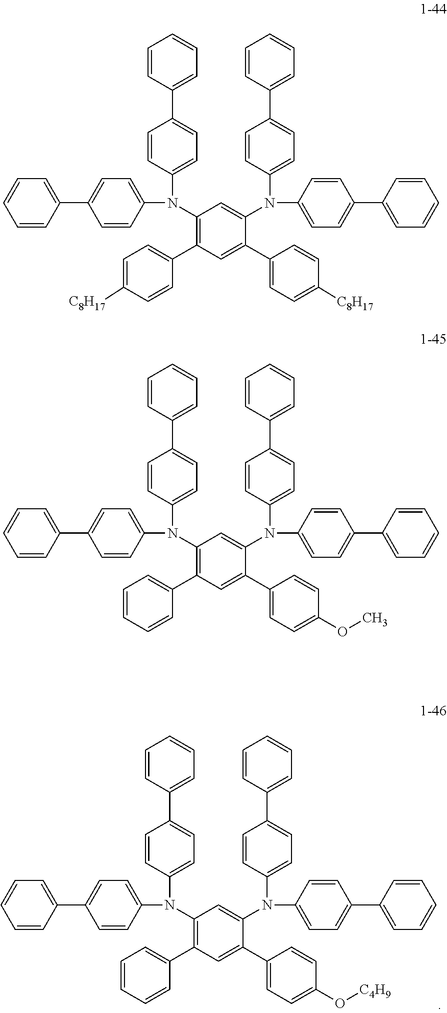

9. The compound for an organic electroluminescent device as claimed in claim 1, wherein the compound includes one of the following Compounds 1-47 to 1-58:

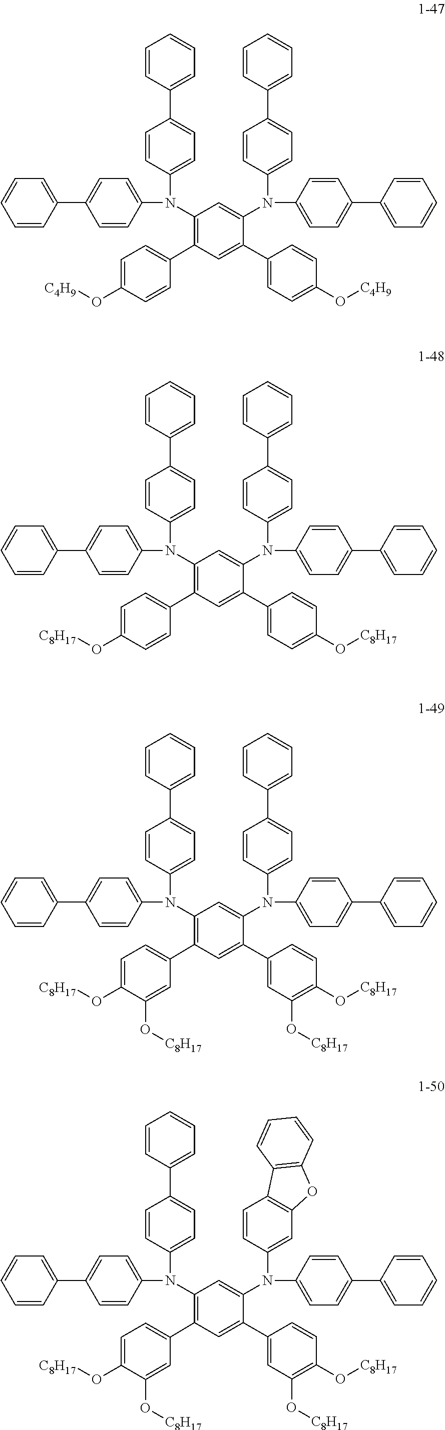

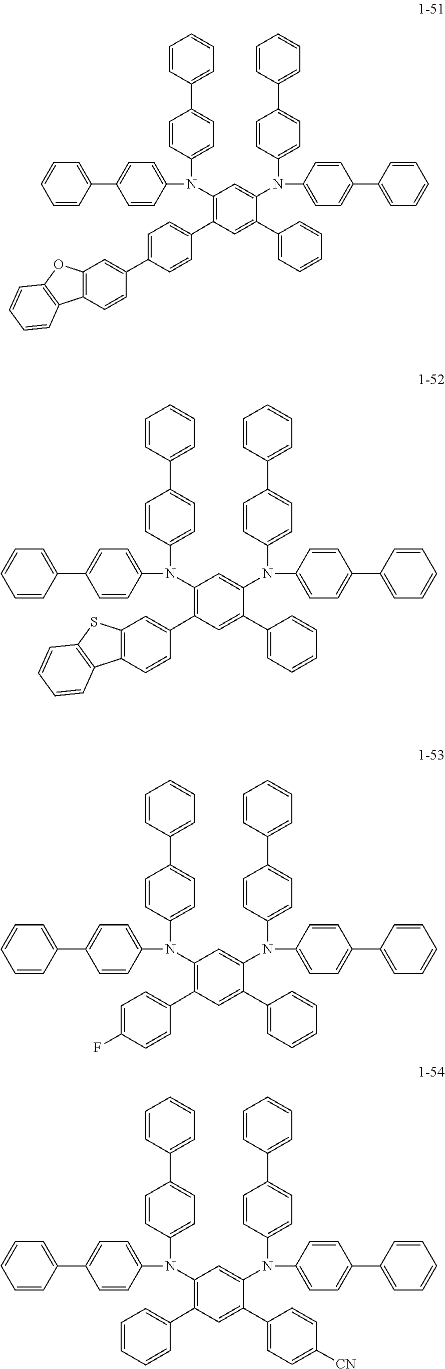

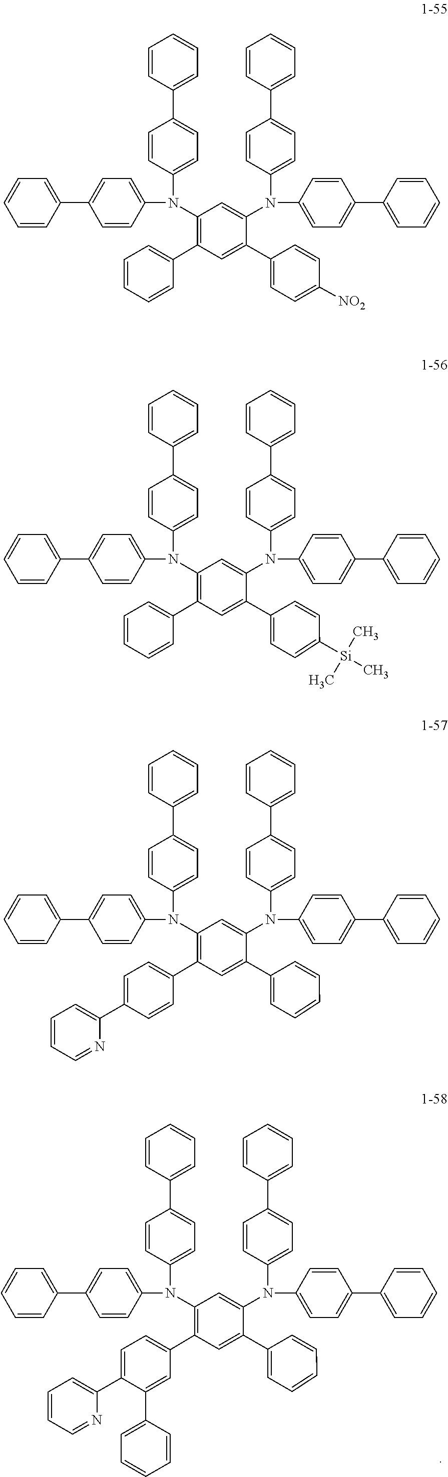

10. An organic electroluminescent device comprising a compound for an organic electroluminescent device, the compound being represented by the following Formula 1: 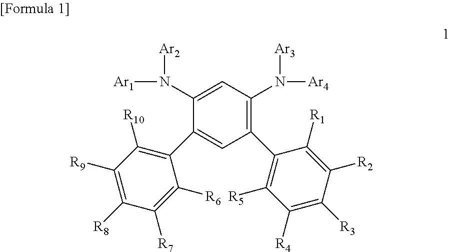
wherein, in Formula 1,
Ar₁ to Ar₄ are each independently a substituted or unsubstituted aryl group or a substituted or unsubstituted heteroaryl group, at least one of Ar₁ to Ar₄ being a substituted or unsubstituted aryl group having at least 10 ring carbon atoms or a substituted or unsubstituted heteroaryl group having at least 10 ring carbon atoms,
R₁ to R₁₀ are each independently a hydrogen atom, a substituted or unsubstituted aryl group, a substituted or unsubstituted heteroaryl group, a substituted or unsubstituted alkyl group, a substituted or unsubstituted alkoxy group, a halogen atom, an electron withdrawing group, or a deuterium atom, and
R₁ to R₁₀ are separate or adjacent ones thereof are combined to each other to form a substituted or unsubstituted ring.

11. The organic electroluminescent device as claimed in claim 10, wherein the aryl group having at least 10 ring carbon atoms is a biphenyl group, a naphthyl group, a phenanthryl group, or a triphenylenyl group.

12. The organic electroluminescent device as claimed in claim 11, wherein the aryl group having at least 10 ring carbon atoms is a biphenyl group.

13. The organic electroluminescent device as claimed in claim 10, wherein the heteroaryl group having at least 10 ring carbon atoms is a dibenzoheterole group.

14. The organic electroluminescent device as claimed in claim 13, wherein the dibenzoheterole group is represented by the following Formula 2: 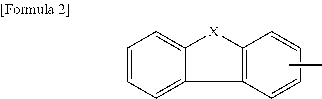
wherein, in Formula 2, X is one of 0, S, or Si(R₁₁)₂, in which each R₁₁ is independently a hydrogen, a substituted or unsubstituted alkyl group, or a substituted or unsubstituted aryl group.

15. The organic electroluminescent device as claimed in claim 10, wherein Ar₁ to Ar₄ are each independently a substituted or unsubstituted aryl group having at least 10 ring carbon atoms or a substituted or unsubstituted heteroaryl group having at least 10 ring carbon atoms.

16. The organic electroluminescent device as claimed in claim 10, wherein the compound includes one of the following Compounds 1-1 to 1-24:

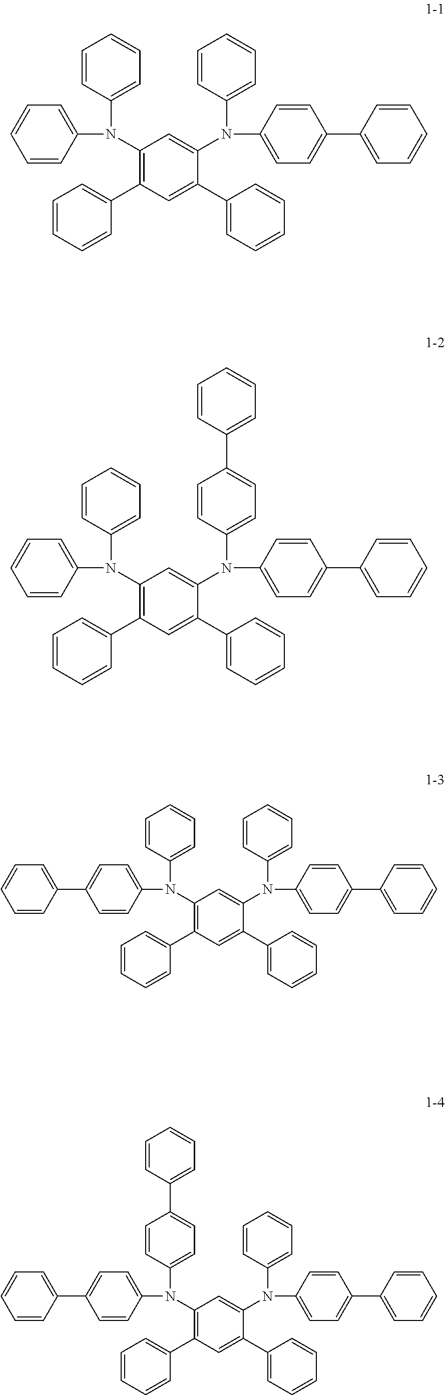 [SMILES: c1ccc(-c2ccc(N(c3ccccc3)c3cc(N(c4ccccc4)c4ccccc4)c(-c4ccccc4)cc3-c3ccccc3)cc2)cc1 ; c1ccc(-c2ccc(N(c3ccc(-c4ccccc4)cc3)c3cc(N(c4ccccc4)c4ccccc4)c(-c4ccccc4)cc3-c3ccccc3)cc2)cc1 ; c1ccc(-c2ccc(N(c3ccccc3)c3cc(N(c4ccccc4)c4ccc(-c5ccccc5)cc4)c(-c4ccccc4)cc3-c3ccccc3)cc2)cc1 ; c1ccc(-c2ccc(N(c3ccccc3)c3cc(N(c4ccc(-c5ccccc5)cc4)c4ccc(-c5ccccc5)cc4)c(-c4ccccc4)cc3-c3ccccc3)cc2)cc1]

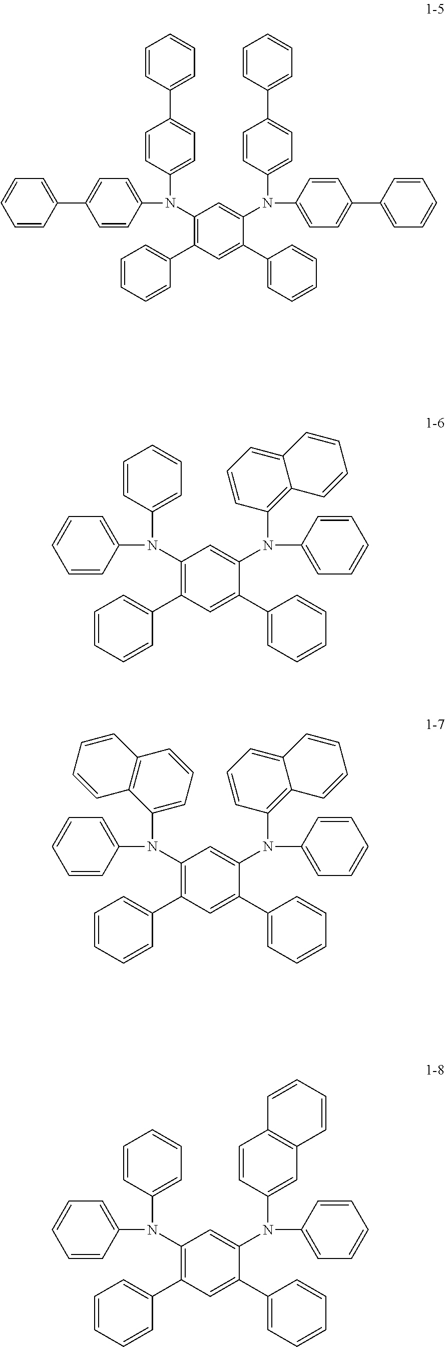 [SMILES: c1ccc(-c2ccc(N(c3ccc(-c4ccccc4)cc3)c3cc(N(c4ccc(-c5ccccc5)cc4)c4ccc(-c5ccccc5)cc4)c(-c4ccccc4)cc3-c3ccccc3)cc2)cc1 ; c1ccc(-c2cc(-c3ccccc3)c(N(c3ccccc3)c3cccc4ccccc34)cc2N(c2ccccc2)c2ccccc2)cc1 ; c1ccc(-c2cc(-c3ccccc3)c(N(c3ccccc3)c3cccc4ccccc34)cc2N(c2ccccc2)c2cccc3ccccc23)cc1 ; c1ccc(-c2cc(-c3ccccc3)c(N(c3ccccc3)c3ccc4ccccc4c3)cc2N(c2ccccc2)c2ccccc2)cc1]

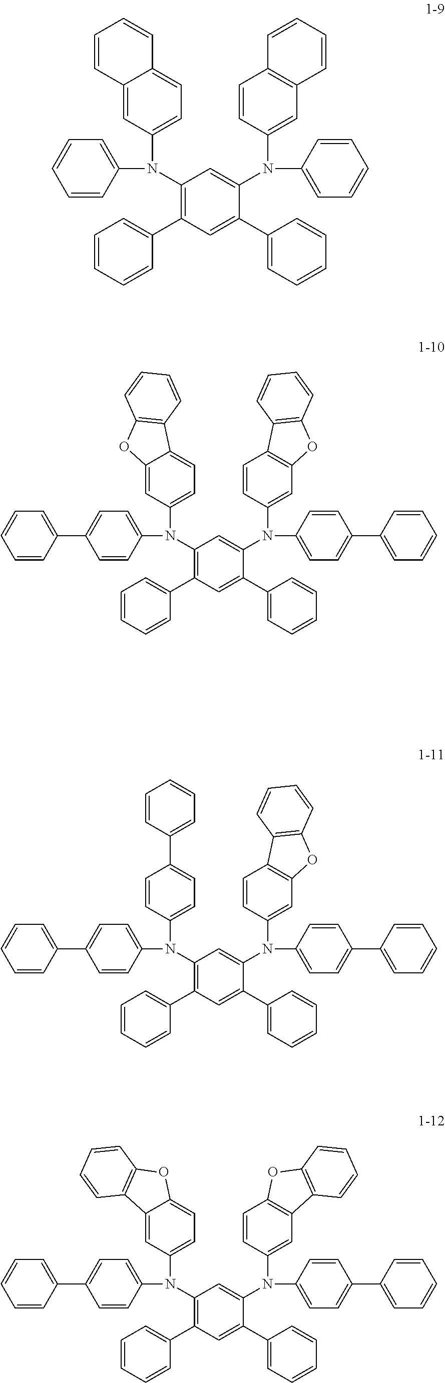 [SMILES: c1ccc(-c2cc(-c3ccccc3)c(N(c3ccccc3)c3ccc4ccccc4c3)cc2N(c2ccccc2)c2ccc3ccccc3c2)cc1 ; c1ccc(-c2ccc(N(c3ccc4c(c3)oc3ccccc34)c3cc(N(c4ccc(-c5ccccc5)cc4)c4ccc5c(c4)oc4ccccc45)c(-c4ccccc4)cc3-c3ccccc3)cc2)cc1 ; c1ccc(-c2ccc(N(c3ccc(-c4ccccc4)cc3)c3cc(N(c4ccc(-c5ccccc5)cc4)c4ccc5c(c4)oc4ccccc45)c(-c4ccccc4)cc3-c3ccccc3)cc2)cc1 ; c1ccc(-c2ccc(N(c3ccc4oc5ccccc5c4c3)c3cc(N(c4ccc(-c5ccccc5)cc4)c4ccc5oc6ccccc6c5c4)c(-c4ccccc4)cc3-c3ccccc3)cc2)cc1]

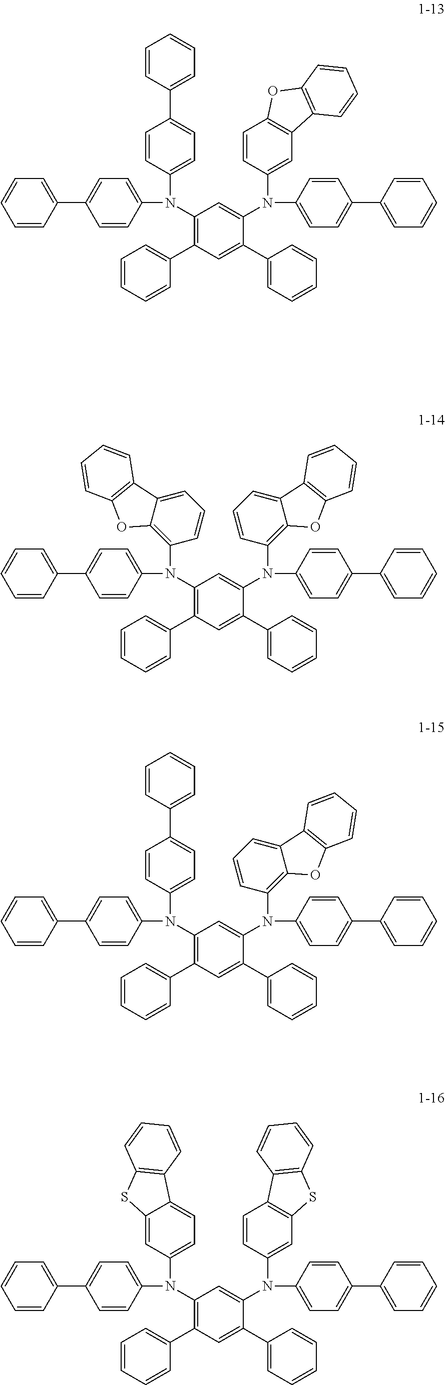 [SMILES: c1ccc(-c2ccc(N(c3ccc(-c4ccccc4)cc3)c3cc(N(c4ccc(-c5ccccc5)cc4)c4ccc5oc6ccccc6c5c4)c(-c4ccccc4)cc3-c3ccccc3)cc2)cc1 ; c1ccc(-c2ccc(N(c3cc(N(c4ccc(-c5ccccc5)cc4)c4cccc5c4oc4ccccc45)c(-c4ccccc4)cc3-c3ccccc3)c3cccc4c3oc3ccccc34)cc2)cc1 ; c1ccc(-c2ccc(N(c3ccc(-c4ccccc4)cc3)c3cc(N(c4ccc(-c5ccccc5)cc4)c4cccc5c4oc4ccccc45)c(-c4ccccc4)cc3-c3ccccc3)cc2)cc1 ; c1ccc(-c2ccc(N(c3ccc4c(c3)sc3ccccc34)c3cc(N(c4ccc(-c5ccccc5)cc4)c4ccc5c(c4)sc4ccccc45)c(-c4ccccc4)cc3-c3ccccc3)cc2)cc1]

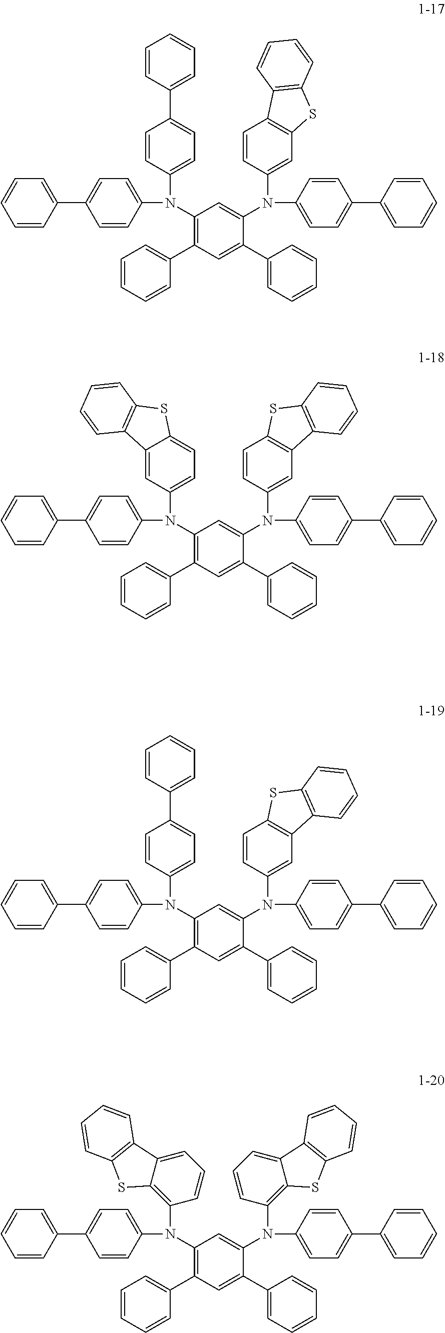 [SMILES: c1ccc(-c2ccc(N(c3ccc(-c4ccccc4)cc3)c3cc(N(c4ccc(-c5ccccc5)cc4)c4ccc5c(c4)sc4ccccc45)c(-c4ccccc4)cc3-c3ccccc3)cc2)cc1 ; c1ccc(-c2ccc(N(c3ccc4sc5ccccc5c4c3)c3cc(N(c4ccc(-c5ccccc5)cc4)c4ccc5sc6ccccc6c5c4)c(-c4ccccc4)cc3-c3ccccc3)cc2)cc1 ; c1ccc(-c2ccc(N(c3ccc(-c4ccccc4)cc3)c3cc(N(c4ccc(-c5ccccc5)cc4)c4ccc5sc6ccccc6c5c4)c(-c4ccccc4)cc3-c3ccccc3)cc2)cc1 ; c1ccc(-c2ccc(N(c3cc(N(c4ccc(-c5ccccc5)cc4)c4cccc5c4sc4ccccc45)c(-c4ccccc4)cc3-c3ccccc3)c3cccc4c3sc3ccccc34)cc2)cc1]

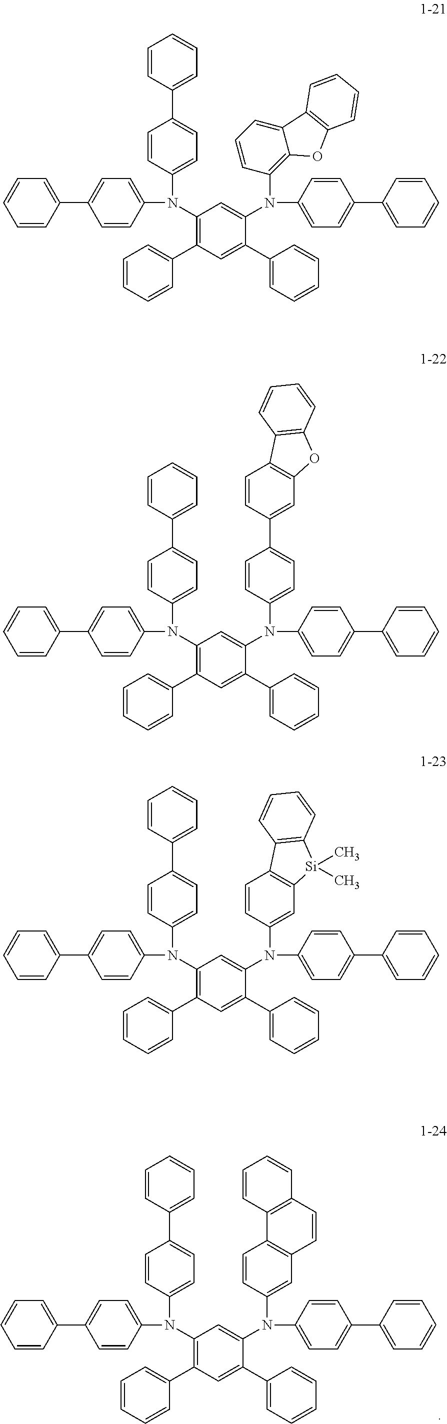 [SMILES: c1ccc(-c2ccc(N(c3ccc(-c4ccccc4)cc3)c3cc(N(c4ccc(-c5ccccc5)cc4)c4cccc5c4oc4ccccc45)c(-c4ccccc4)cc3-c3ccccc3)cc2)cc1 ; c1ccc(-c2ccc(N(c3ccc(-c4ccccc4)cc3)c3cc(N(c4ccc(-c5ccccc5)cc4)c4ccc(-c5ccc6c(c5)oc5ccccc56)cc4)c(-c4ccccc4)cc3-c3ccccc3)cc2)cc1 ; C[Si]1(C)c2ccccc2-c2ccc(N(c3ccc(-c4ccccc4)cc3)c3cc(N(c4ccc(-c5ccccc5)cc4)c4ccc(-c5ccccc5)cc4)c(-c4ccccc4)cc3-c3ccccc3)cc21 ; c1ccc(-c2ccc(N(c3ccc(-c4ccccc4)cc3)c3cc(N(c4ccc(-c5ccccc5)cc4)c4ccc5c(ccc6ccccc65)c4)c(-c4ccccc4)cc3-c3ccccc3)cc2)cc1]

17. The organic electroluminescent device as claimed in claim 10, wherein the compound includes one of the following Compounds 1-25 to 1-46:

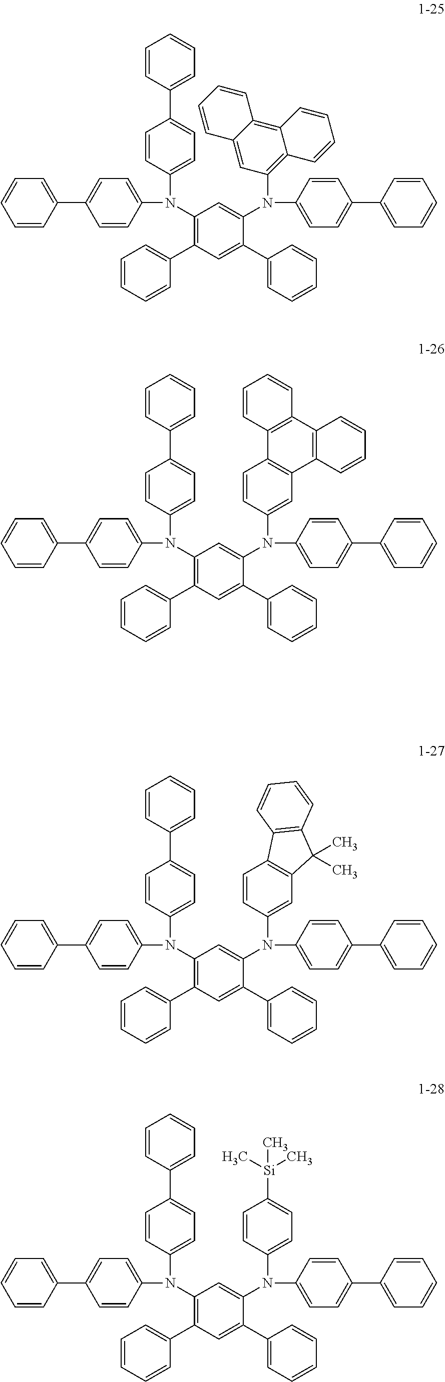 [SMILES: c1ccc(-c2ccc(N(c3ccc(-c4ccccc4)cc3)c3cc(N(c4ccc(-c5ccccc5)cc4)c4cc5ccccc5c5ccccc45)c(-c4ccccc4)cc3-c3ccccc3)cc2)cc1 ; c1ccc(-c2ccc(N(c3ccc(-c4ccccc4)cc3)c3cc(N(c4ccc(-c5ccccc5)cc4)c4ccc5c6ccccc6c6ccccc6c5c4)c(-c4ccccc4)cc3-c3ccccc3)cc2)cc1 ; CC1(C)c2ccccc2-c2ccc(N(c3ccc(-c4ccccc4)cc3)c3cc(N(c4ccc(-c5ccccc5)cc4)c4ccc(-c5ccccc5)cc4)c(-c4ccccc4)cc3-c3ccccc3)cc21 ; C[Si](C)(C)c1ccc(N(c2ccc(-c3ccccc3)cc2)c2cc(N(c3ccc(-c4ccccc4)cc3)c3ccc(-c4ccccc4)cc3)c(-c3ccccc3)cc2-c2ccccc2)cc1]

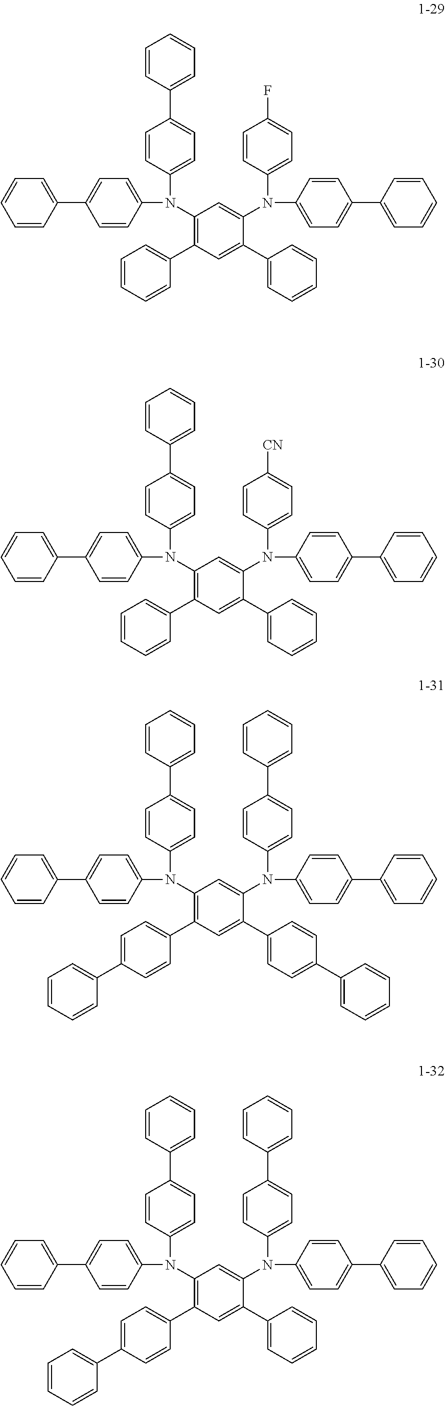 [SMILES: Fc1ccc(N(c2ccc(-c3ccccc3)cc2)c2cc(N(c3ccc(-c4ccccc4)cc3)c3ccc(-c4ccccc4)cc3)c(-c3ccccc3)cc2-c2ccccc2)cc1 ; N#Cc1ccc(N(c2ccc(-c3ccccc3)cc2)c2cc(N(c3ccc(-c4ccccc4)cc3)c3ccc(-c4ccccc4)cc3)c(-c3ccccc3)cc2-c2ccccc2)cc1 ; c1ccc(-c2ccc(-c3cc(-c4ccc(-c5ccccc5)cc4)c(N(c4ccc(-c5ccccc5)cc4)c4ccc(-c5ccccc5)cc4)cc3N(c3ccc(-c4ccccc4)cc3)c3ccc(-c4ccccc4)cc3)cc2)cc1 ; c1ccc(-c2ccc(-c3cc(-c4ccccc4)c(N(c4ccc(-c5ccccc5)cc4)c4ccc(-c5ccccc5)cc4)cc3N(c3ccc(-c4ccccc4)cc3)c3ccc(-c4ccccc4)cc3)cc2)cc1]

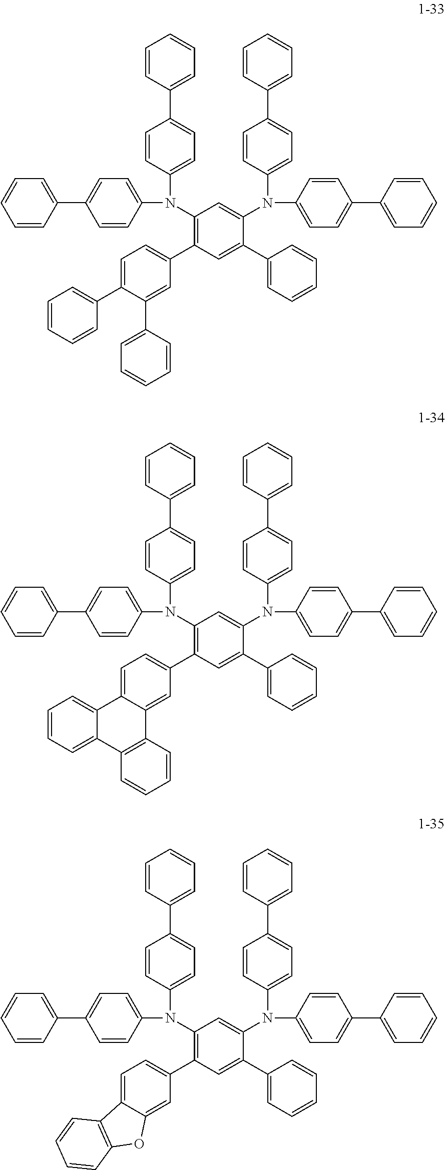 [SMILES: c1ccc(-c2ccc(N(c3ccc(-c4ccccc4)cc3)c3cc(N(c4ccc(-c5ccccc5)cc4)c4ccc(-c5ccccc5)cc4)c(-c4ccc(-c5ccccc5)c(-c5ccccc5)c4)cc3-c3ccccc3)cc2)cc1 ; c1ccc(-c2ccc(N(c3ccc(-c4ccccc4)cc3)c3cc(N(c4ccc(-c5ccccc5)cc4)c4ccc(-c5ccccc5)cc4)c(-c4ccc5c6ccccc6c6ccccc6c5c4)cc3-c3ccccc3)cc2)cc1 ; c1ccc(-c2ccc(N(c3ccc(-c4ccccc4)cc3)c3cc(N(c4ccc(-c5ccccc5)cc4)c4ccc(-c5ccccc5)cc4)c(-c4ccc5c(c4)oc4ccccc45)cc3-c3ccccc3)cc2)cc1]

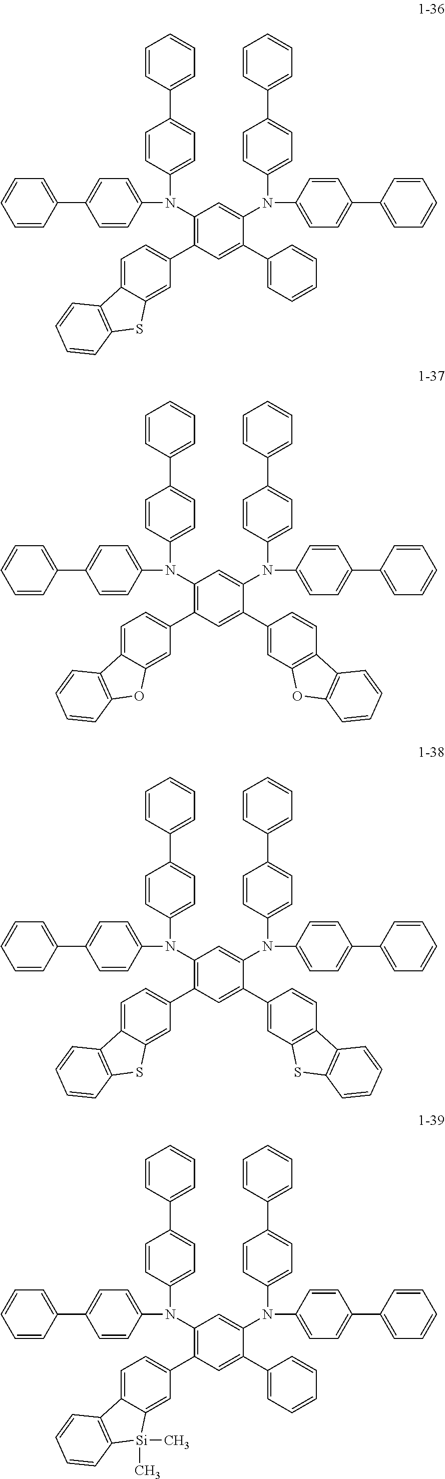

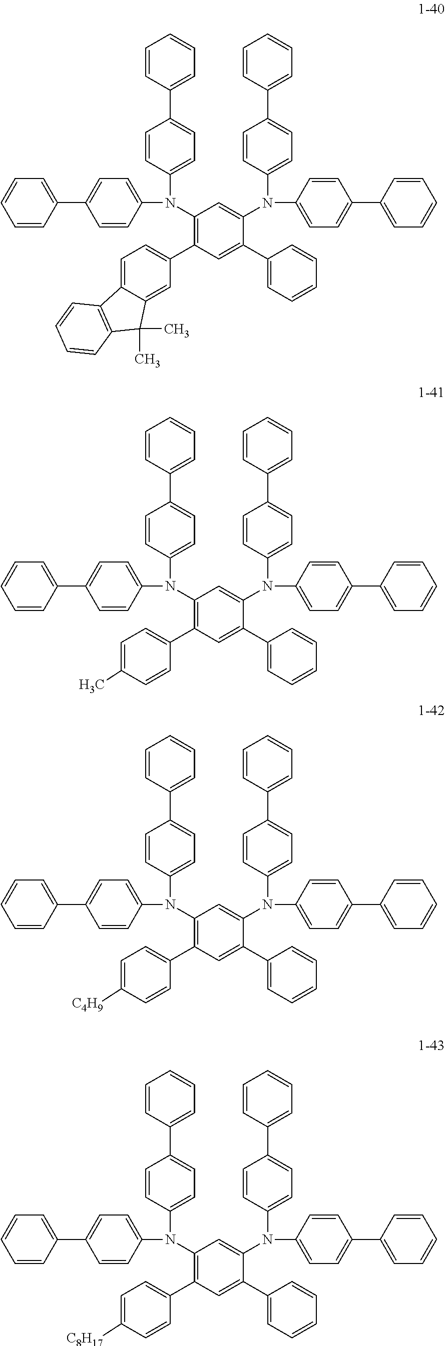

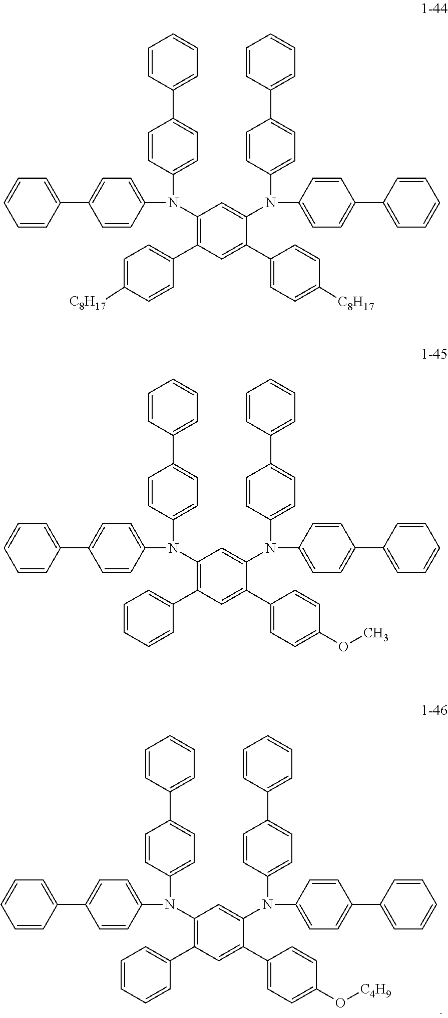

18. The organic electroluminescent device as claimed in claim 10, wherein the compound includes one of the following Compounds 1-47 to 1-58:

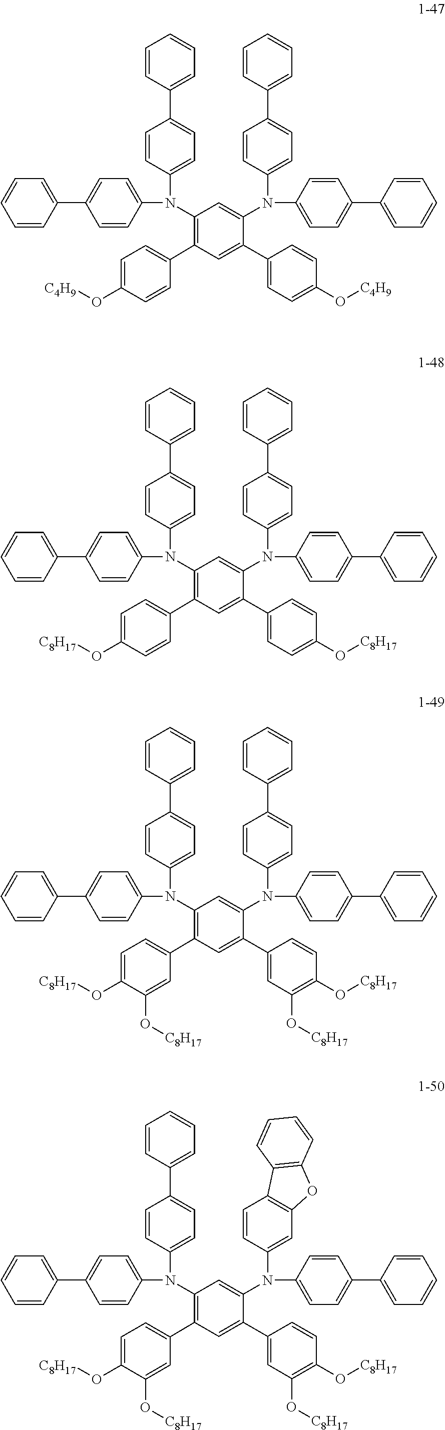

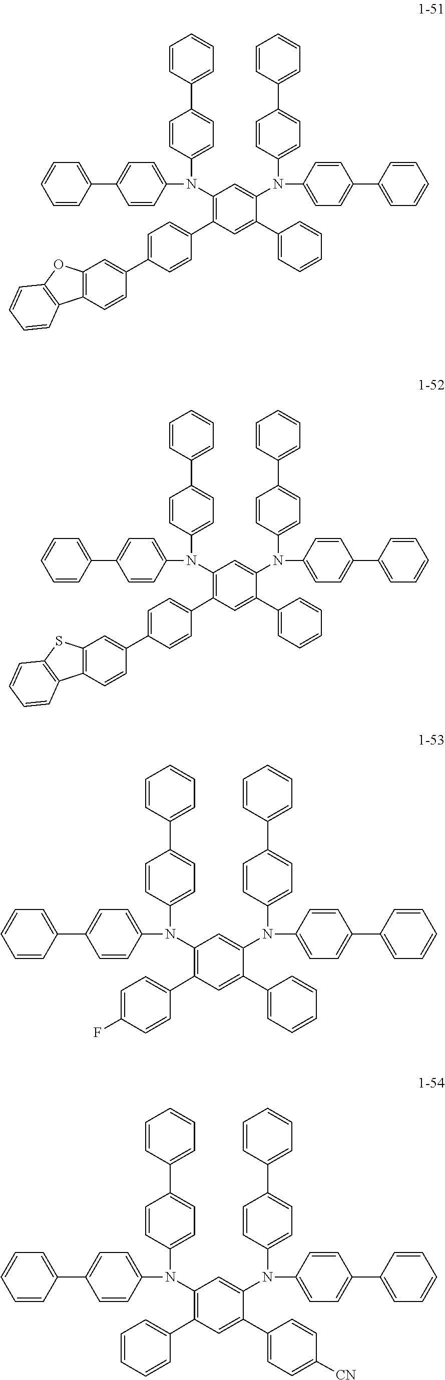

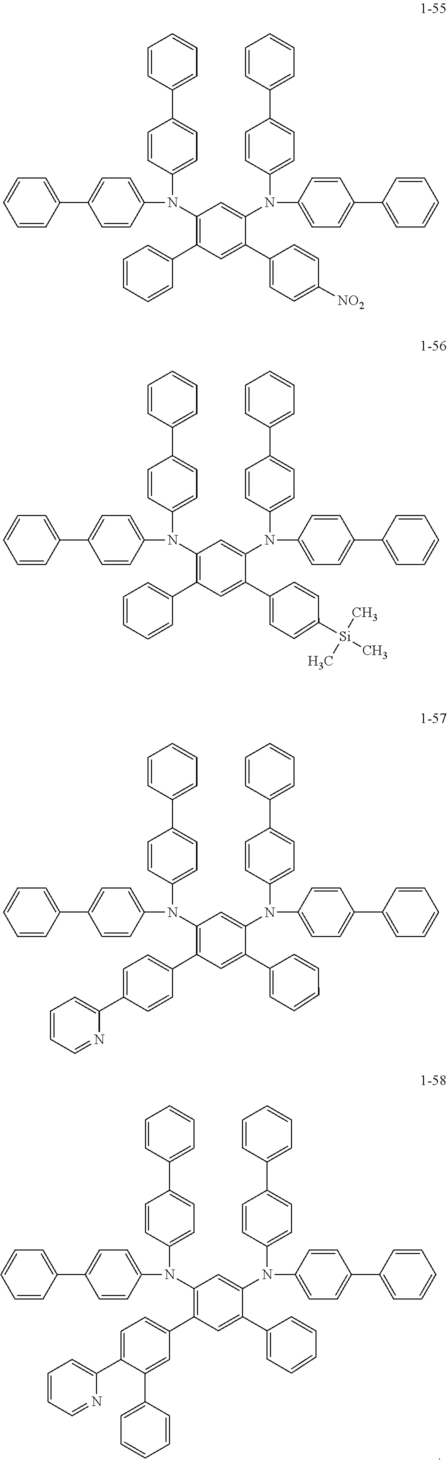

19. The organic electroluminescent device as claimed in claim 10, wherein:
the organic electroluminescent device includes a hole transport layer, and
the compound for an organic electroluminescent device is included in the hole transport layer.
